(19) United States
(12) Patent Application Publication
(10) Pub. No.: US 2025/0195440 A1
(43) Pub. Date: Jun. 19, 2025

(54) PHARMACEUTICAL FORMULATION COMPRISING GLUCOKINASE ACTIVATOR AND USE THEREOF

(71) Applicant: Hua Medicine USA Inc., Edison, NJ (US)

(72) Inventors: Zhongjie PENG, Edison, NJ (US); Fuxing TANG, Edison, NJ (US); Li CHEN, Shanghai (CN)

(21) Appl. No.: 18/980,984

(22) Filed: Dec. 13, 2024

Related U.S. Application Data
(60) Provisional application No. 63/609,970, filed on Dec. 14, 2023.

(51) Int. Cl. A61K 9/24; A61K 9/00; A61K 9/20; A61K 9/28; A61K 31/4439; A61P 3/10

(52) U.S. Cl. CPC A61K 9/209; A61K 9/0053; A61K 9/2009; A61K 9/2013; A61K 9/2054; A61K 9/284; A61K 9/2853; A61K 31/4439; A61P 3/10

(57) ABSTRACT

Provided herein are pharmaceutical formulations comprising glucokinase activator, or a prodrug, or a pharmaceutically acceptable salt, an isotope labeled analogue, a crystalline form, a hydrate, a solvate, or a diastereomeric or enantiomeric form thereof and the use thereof for treating diseases; the pharmaceutical formulations are in the form of immediate-release formulations, extended-release formulations, or combination of immediate-release formulation and extended-release formulations. The pharmaceutical formulations achieved once-a-day therapy for some diseases, in particular, type II Diabetes Mellitus with obesity. The pharmaceutical formulations exhibit sustained 24 hours of glucose-lowering effects. The pharmaceutical formulations also achieved lower Cₘₐₓ, extended T_{1/2} as well as maintained similar bioavailability and increased MRT compared with commercial Dorzagliatin tablet, enhanced the pharmacology effect of restoring the glucose stimulated GLP-1 secretion in diabetes with obesity.

### CROSS-REFERENCE TO RELATED APPLICATIONS

This application claims priority to U.S. Provisional Application No. 63/609,970, filed on Dec. 14, 2023, the entirety of which is incorporated herein by reference.

### FIELD

The present disclosure provides pharmaceutical formulations comprising glucokinase activator, or a prodrug, or a pharmaceutically acceptable salt, an isotope labeled analogue, a crystalline form, a hydrate, a solvate, or a diastereomeric or enantiomeric form thereof and the use thereof for treating diseases. The pharmaceutical formulations are in the form of immediate-release formulations, extended-release formulations, or combination of immediate-release formulation and extended-release formulation. The pharmaceutical formulations of the present disclosure exhibit sustained 24 hours of glucose-lowering effects.

### BACKGROUND

Glucokinase (GK, hexokinase IV) is one of the four kinds of hexokinases of mammals. Hexokinase is an enzyme in the first stage of glycolysis system and catalyzes the reaction from glucose to glucose-hexaphosphate. GK is mainly found in pancreatic β-cells and liver parenchymal cells and serves as a rate-controlling enzyme for glucose metabolism in pancreatic β-cells and liver parenchymal cells, which play an important role in whole-body glucose homeostasis.

GK, which acts as a glucose sensor in blood glucose homeostasis, senses blood glucose changes, regulates the secretion of glucose-controlling hormones, insulin, glucagon, and GLP-1, and constitutes a sensing system for regulation of blood glucose homeostasis in human body. GK is mainly expanded in the liver, where it rapidly converts glucose into hepatic glycogen for storage in response to elevated blood glucose, while lowering the glucose level in the blood. Glucose reserve during glucose ingestion and glucose supply during fasting, controlled by glucose-controlling hormones, constituted the major part of regulation of blood glucose homeostasis in human body.

GLP-1 and glucagon-like peptide-1 receptor agonist (GLP-1RA) have been studied intensively for the treatment of diabetes and obesity through peripheral and central receptors. Current therapies have been evolved into a dual active peptide of GLP-1 and glucose-dependent insulinotropic peptide (GIP). GLP-1 originated from intestinal L cell and pancreatic alpha cell, together with its circulating metabolites generated by dipeptidyl peptidase 4 (DPP-4) enzyme, were considered to play an important role in glucose homeostasis through glucagon-like peptide-1 receptor (GLP-1R), as well as having cardiovascular and neurological protection effects through non-GLP-1R regulated functions. Therefore, the strategy of developing GLP-1R-based therapy may have limited the physiological function of incretins and its metabolites beyond their role in glucose homeostasis. Defects of GLP-1 secretion in response to glucose challenge have been reported in western and eastern patients with impaired glucose tolerance (IGT) and type 2 diabetes (T2D), which can be rescued by either GLP-1RA, or ideally a glucose-dependent regulator on GLP-1 secretion (Nature Communications 14, 1405 (2023)).

In a phase I open-label clinical trial study of Dorzagliatin and sitagliptin in patients with type II diabetes and obesity, the results showed that Dorzagliatin alone improved the impaired glucose-stimulated GLP-1 secretion approaching to the healthy range of maximum GLP-1 level (Nature Communications 14, 1405 (2023)).

Glucokinase activator (GKA) was developed against the characteristics of GK as a target, which can improve the secretion function of insulin, glucagon and GLP-1 for glucose regulation by increasing the sensitivity of α, β and L cells. There is a demand for glucokinase activators in the field of diabetes, in particular, Type 2 Diabetes Mellitus with obesity.

WO2009/127546A1 discloses glucokinase activators of pyrrolidone series, in particular, (S)-2-[4-(2-chloro-phenoxy)-2-oxo-2,5-dihydro-pyrrol-1-yl]-4-methyl-pentanoic acid [1-((R)-2,3-dihydroxy-propyl)-1H-pyrazol-3-yl]-amide, also known to as Dorzagliatin or HMS5552.

WO2023/040937A1 discloses prodrugs of pyrrolidone derivatives as glucokinase activator, in particular, the prodrug of Dorzagliatin of formula (I):

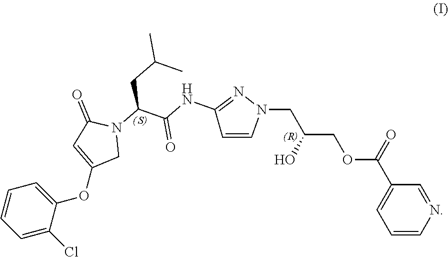

Studies in WO2023/040937A1 showed that the compound of formula (I) was quickly and completely converted into active Dorzagliatin with esterase enzymes in gastrointestinal tract.

Currently, Dorzagliatin is commercially available in the form of tablet under the product name HuaTangNing in China as a first-in-class GKA product over the world. The product is a solid pharmaceutical dosage form containing 75 mg of Dorzagliatin per tablet and is administered twice daily (BID): at a single dose of 75 mg in the morning and a single dose of 75 mg in the evening.

Multiple dosing of tablets may result in decreased patient compliance, the potential reduction of daily doses (to once a day) products can improve patient compliance and help to avoid missed doses. Thus, there is an existing and continual need for a once-a-day formulation containing GKA or a pharmaceutically acceptable salt that provides dosing convenience and increases patient compliance.

### SUMMARY

Since there is an existing and continual need for a once-a-day pharmaceutical formulation containing GKA, the inventors of the present disclosure developed immediate-release formulations, or extended-release formulations, or combination of immediate-release and extended-release formulation of GKA (e.g., Dorzagliatin, prodrug of Dorzagliatin).

The pharmaceutical formulations of the present disclosure achieved once-a-day therapy of GKA, or a prodrug for some diseases (e.g., diabetes, diabetes with obesity), and achieved lower Cₘₐₓ, extended T_{1/2} as well as maintained similar bioavailability and increased mean residence time (MRT) compared with Dorzaglatin commercial product (HuaTangNing).

Further, the pharmaceutical formulations of the present disclosure enhanced the pharmacology effect of Dorzagliatin in restoring the glucose stimulated GLP-1 secretion in diabetes and obesity.

The present disclosure provides a pharmaceutical formulation comprising glucokinase activator, or a prodrug a pharmaceutically acceptable salt, an isotope labeled analogue, a crystalline form, a hydrate, a solvate, or a diastereomeric or enantiomeric form thereof.

Specifically, the present disclosure provides a pharmaceutical formulation, comprising:
(a) glucokinase activator, or a prodrug, or a pharmaceutically acceptable salt, an isotope labeled analogue, a crystalline form, a hydrate, a solvate, or a diastereomeric or enantiomeric form thereof;
(b) optionally one or more excipients;
wherein the pharmaceutical formulation is an immediate-release formulation.

Alternatively, wherein the prodrug is a prodrug of Dorzagliatin, or a pharmaceutically acceptable salt, an isotope labeled analogue, a crystalline form, a hydrate, a solvate, or a diastereomeric or enantiomeric form thereof.

Also, the present disclosure provides a pharmaceutical formulation, comprising at least two parts, wherein the first part is the immediate-release pharmaceutical formulation as defined in the present context; and the second part is an extended-release formulation, comprising: glucokinase activator, or a prodrug, or a pharmaceutically acceptable salt, an isotope labeled analogue, a crystalline form, a hydrate, a solvate, or a diastereomeric or enantiomeric form thereof; and release modifier(s);

alternatively, wherein the prodrug is a prodrug of Dorzagliatin, or a pharmaceutically acceptable salt, an isotope labeled analogue, a crystalline form, a hydrate, a solvate, or a diastereomeric or enantiomeric form thereof.

Also, the present disclosure provides a bilayer formulation of compound of formula (I) (once-a-day), comprising a combination of an immediate-release and an extended-release formulation as defined in the present context. The immediate-release formulation allows fast release of API that is important for diabetes patient for a fast onset action of lowering the blood glucose, and the extended-release formulation minimizes the fluctuation of drug concentration in the blood comparing to product with BID doses, consequently better therapeutic efficacy and less side effect. The once-a-day formulation provides dosing convenience and increases patient compliance.

The pharmaceutical formulations of the present disclosure exhibit sustained 24 hours of glucose-lowering effects.

In another aspect, the present disclosure provides the pharmaceutical formulations as defined in the present context in the manufacture of a medicament for preventing, slowing the progression of, delaying, or treating one or more metabolic disorders selected from the group consisting of: diabetes (including type I, type II diabetes, type II diabetes with obesity, and cystic fibrosis-related diabetes (CFRD)), diabetic kidney disease, impaired glucose tolerance, impaired fasting blood glucose, hyperglycemia, postprandial hyperglycemia, overweight, obesity, hypertension, insulin resistance, diseases associated with islet dysfunction and/or metabolic syndrome; or improving blood glucose control and/or reducing fasting plasma glucose, postprandial plasma glucose and/or glycosylated hemoglobin HbA1c; or preventing, slowing, delaying, or reversing complications of diabetes mellitus; or curing diabetes, causing diabetes remission or regressing diabetes.

In another aspect, the present disclosure provides methods of treating diseases or disorders associated with GK activity comprising administering to a mammal, e.g., a human, in need of such treatment a therapeutically effective amount of the pharmaceutical formulation described herein, and optionally a film coated tablet. The pharmaceutical formulations of the present disclosure, as described herein, can be administered to a mammal, e.g., a human, for preventing, slowing the progression of, delaying, or treating one or more metabolic disorders selected from the group consisting of: diabetes (including type I diabetes, type II diabetes, type II diabetes with obesity, and cystic fibrosis-related diabetes (CFRD)), diabetic kidney disease, impaired glucose tolerance, impaired fasting blood glucose, hyperglycemia, postprandial hyperglycemia, overweight, obesity, hypertension, insulin resistance, diseases associated with islet dysfunction and/or metabolic syndrome; or improving blood glucose control and/or reducing fasting plasma glucose, postprandial plasma glucose and/or glycosylated hemoglobin HbA1c; or preventing, slowing, delaying, or reversing complications of diabetes mellitus; or curing diabetes, causing diabetes remission or regressing diabetes.

In some embodiments, the pharmaceutical formulation of the present disclosure is administered to humans for preventing, slowing the progression of, delaying, or treating type II diabetes with obesity.

In another aspect, the therapeutically effective amount of the compound of formula (I) in the pharmaceutical formulation is administered once-a-day.

Other objects of the present disclosure will be apparent to those skilled in the art from the description and examples.

### BRIEF DESCRIPTION OF THE DRAWINGS

FIG. 1 shows the in vitro dissolution profiles comparison of film coated tablets.

FIG. 2 shows the in vitro dissolution profiles comparison of tablets A-E.

FIG. 2-1 shows the in vitro dissolution profiles comparison of tablets G-O.

FIG. 3 shows the PK profiles comparison of tablets A-E and tablet K.

FIG. 4 shows the blood glucose profiles comparison of tablets A-E and tablet K.

### DETAILED DESCRIPTION

### Definitions

Unless otherwise specified, all technical and scientific terms used herein have the same meanings as commonly understood by those skilled in the art to which the present disclosure belongs, but in case of conflict, the definitions in this specification shall prevail.

As used in the specification and claims, the singular forms “a”, “an” and “the (said)” include plural forms, unless clearly specified otherwise in the context.

All numerical values or expressions related to component amounts used in the specification and claims should be understood to be modified by “about” in all cases. The term “about” when referring to an amount or a numerical range means that the amount or the numerical range referred to is an approximate value within experimental variability (or within statistical experimental error). Therefore, the amount or the numerical range can be varied between, for example, ±5% of the amount or the numerical range referred to.

As used in this specification and in the claims, “and/or” should be understood to mean “either or both” of the associated elements, i.e., the elements exist jointly in some cases and separately in other cases. Multiple elements listed with “and/or” should be construed in the same fashion, i.e., “one or more” of the associated elements. In addition to the elements specifically identified by the “and/or” clause, other elements may optionally exist, whether related or unrelated to those elements specifically identified. Thus, as a non-limiting example, a reference to “A and/or B”, when used in conjunction with open-ended language such as “comprising”, can refer, in one embodiment, to A only (optionally including elements other than B); in another embodiment, to B only (optionally including elements other than A); in yet another embodiment, to both A and B (optionally including other elements); etc.

Abbreviations used herein have their usual meanings in the fields of chemistry, biology and formulation.

The term “prodrug” means a compound that, after administration, may be converted into the parent drug by various mechanisms, including enzymatic processes and metabolic hydrolysis. To produce a prodrug, a pharmaceutically active compound (or a suitable precursor thereof) is modified chemically such that the modified form is less active or inactive, but the chemical modification is effectively reversible under certain enzymatic processes and metabolic hydrolysis.

The glucokinase activators, or the prodrug of the present disclosure contain one or more asymmetric centers and thus may exist in various stereoisomeric forms, e.g., enantiomeric and/or diastereomeric forms. For example, the glucokinase activators of the present disclosure may be individual enantiomers, diastereomers or geometric isomers (such as cis and trans isomers), or may be in the form of mixtures of stereoisomers comprising racemic mixtures and mixtures enriched in one or more stereoisomers. Isomers can be separated from mixtures by methods known to those skilled in the art, including chiral high pressure liquid chromatography (HPLC) and formation and crystallization of chiral salts; or alternative isomers may be prepared by asymmetric synthesis.

The present disclosure also comprises compounds that are labeled with isotopes, which are equivalent to the prodrug, but one or more atoms are replaced by atoms having an atom mass or mass number that are different from that of atoms that are common in nature. Examples of isotopes which may be introduced into the compounds of the disclosure include isotopes of hydrogen, carbon, nitrogen, oxygen, phosphorus, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F and ³⁶Cl, respectively. Compounds of the present disclosure that comprise the above isotopes and/or other isotopes of other atoms, prodrugs thereof and pharmaceutically acceptable salts of said compounds or prodrugs all are within the scope of the present disclosure. Certain isotope-labeled compounds of the present disclosure, such as those incorporating radioactive isotopes (e.g., ³H and ¹⁴C), can be used for the measurement of the distribution of drug and/or substrate in tissue. Tritium, which is ³H and carbon-14, which is ¹⁴C isotope, are yet alternative, because they are easy to be prepared and detected. Furthermore, replaced by heavier isotopes, such as deuterium, which is ²H, may provide therapeutic benefits due to the higher metabolic stability, such as prolonging the half-life in vivo or decreasing the dosage requirements, and thus may be alternative in some cases. Isotope-labeled compounds of glucokinase activator of the present disclosure can be prepared generally by using readily available isotope-labeled reagents to replace non-isotope-labeled reagents in the following schemes and/or the procedures disclosed in the examples and preparation examples.

Compounds of the present disclosure may be in an amorphous or a crystalline form (polymorph). Furthermore, the compounds of the present disclosure may exist in one or more crystalline forms. Therefore, the present disclosure includes all amorphous or crystalline forms of the compounds of the present disclosure within its scope. The term “crystalline form” refers to a crystalline form of a compound (or a salt, hydrate or solvate thereof) in a particular crystal packing arrangement. All crystalline forms have the same elemental composition. Different crystalline forms generally have different X-ray diffraction patterns, infrared spectra, melting points, density, hardness, crystal shapes, optical and electrical properties, stability, and solubility.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The term “solvate” refers to a complex or aggregate formed by one or more molecules of a solute, e.g., a compound provided herein, and one or more molecules of a solvent, which are present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The term “formulation”, as used herein, refers to a blend, aggregation, solution or other combination of materials which includes an active pharmaceutical ingredient (API) which formulation may be adapted to a particular mode of administration by methods known to those of ordinary skill in the art, for example, a formulation suitable for pressing into tablets designed for oral administration, in the treatment, management, prevention and etc. of a disease state or condition in a patient.

By “pharmaceutically available” or “pharmaceutically acceptable”, it is meant a material which is not biologically or otherwise undesirable, i.e., the material may be administered to an individual without causing any undesirable biological effects or interacting in a deleterious manner with any other component of a composition comprising the material.

The term “pharmaceutically acceptable salt” refers to a salt that does not irritate an organism significantly and retains the biological activity and properties of a compound.

The term “pharmaceutically acceptable carrier” refers to an inactive ingredient that does not irritate an organism significantly and does not abrogate the biological activity and properties of an administered compound. As used herein, “carrier” and “excipient” have the same meaning.

The term “immediate-release” (IR) formulation or “immediate-release” part (layer), as used herein and unless otherwise specified, means that an API is released quickly after administration (usually in the stomach for oral administration), quickly reaches peak plasma concentration, and is then metabolized and excreted. In one embodiment, an IR formulation/part/layer provided herein does not comprise a release modifier provided herein.

The term “extended-release” (ER) formulation or “extended-release” part (layer), as used herein and unless otherwise specified, means that an API is released over a prolonged period of time or to maintain certain level of API in the blood circulation for a targeted period of time. In one embodiment, an ER formulation provided herein comprises a release modifier provided herein.

The term “release modifier”, as used herein and unless otherwise specified, refers to a substance that is intended to and also serves to modify the spatial and/or temporal (where and when) release of the API after administration. The modified release can be delayed release (to control where the API is released, such as the small intestine or colon), extended release (the API is released over a prolonged period of time), and/or targeted release (delivering API to a specific organ or tissue). Accordingly, the release modifier can be delayed release modifier, extended release modifier, and/or targeted release modifier.

The terms “effective amount” or “therapeutically effective amount” refer to an amount of an agent sufficient to provide a desired biological result. The result may be reduction and/or alleviation of a sign, symptom, or cause of a disease, or any other desired change of a biological system. For example, a “therapeutically effective amount” for therapeutic use refers to a necessary amount of a composition comprising a compound disclosed herein as an active ingredient for providing a clinically significant decrease in a disease. In any individual case, an appropriate “therapeutically effective amount” may be determined by one of ordinary skilled in the art using routine experimentation. Thus, the expression “therapeutically effective amount” generally refers to an amount of an active substance at which it has a therapeutic effect.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The term “subject” refers to an animal, encompasses mammals and non-mammals, Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like.

In some embodiments, “subject” refers to a mammal, e.g., a human, who has been the object of treatment, observation or experiment. When a human subject suffering from the condition to be treated is included in the activity they are alternatively referred to herein as a “patient”. The term “subject” includes a confirmed patient, but the “subject” does not need to have any special identity to a hospital, clinic, or research facility (e.g., as a confirmed patient, study participant, etc.). In some embodiments, the subject is a human in need of activation of glucokinase.

### DETAILED EMBODIMENTS

The present disclosure provides a pharmaceutical formulation comprising glucokinase activator, or a prodrug, or a pharmaceutically acceptable salt, an isotope labeled analogue, a crystalline form, a hydrate, a solvate, or a diastereomeric or enantiomeric form thereof and the use thereof for treating diseases. The pharmaceutical formulations are in the form of immediate-release formulations, or extended-release formulations, or combination of immediate-release formulation and extended-release formulation. In some embodiments, the pharmaceutical formulations of the present disclosure exhibit sustained 24 hours of glucose-lowering effects.

The present disclosure provides a pharmaceutical formulation, comprising:
(a) glucokinase activator, or a prodrug, or a pharmaceutically acceptable salt, an isotope labeled analogue, a crystalline form, a hydrate, a solvate, or a diastereomeric or enantiomeric form thereof;
(b) optionally one or more excipients.

In some embodiments, the glucokinase activator is Dorzagliatin, or a pharmaceutically acceptable salt, an isotope labeled product, a crystalline form, a hydrate, a solvate, or a diastereomeric or enantiomeric form thereof.

In some embodiments, the glucokinase activator is Dorzagliatin having the structure shown below:

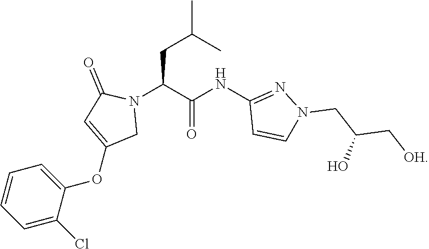 [SMILES: CC(C)C[C@@H](C(=O)Nc1ccn(C[C@@H](O)CO)n1)N1CC(Oc2ccccc2Cl)=CC1=O]

In some embodiments, the glucokinase activator is one disclosed in U.S. Pat. No. 7,741,327 B2 and WO2009127546A1, the disclosure of each of which is incorporated herein by reference in its entirety.

In some embodiments, the prodrug is a prodrug of Dorzagliatin, or a pharmaceutically acceptable salt, an isotope labeled product, a crystalline form, a hydrate, a solvate, or a diastereomeric or enantiomeric form thereof.

In some embodiments, the prodrug of Dorzagliatin is the compound having the structure shown in formula (I):

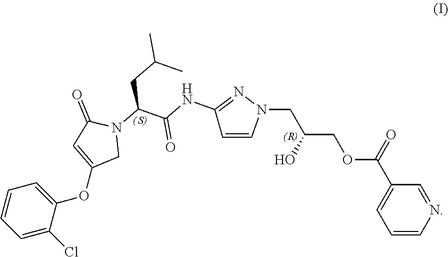

In some embodiments, the compound of formula (I) (free base) is used in the pharmaceutical formulation or methods provided herein. In some embodiments, a pharmaceutically acceptable salt of the compound of formula (I) is used in the pharmaceutical formulation or methods provided herein. In some embodiments, the compound of formula (I) or a pharmaceutically acceptable salt may exist in both amorphous and crystalline forms. In various embodiments of the invention, the compound of formula (I) can be present in either amorphous or crystalline forms or as a mixture of amorphous and crystalline forms.

In some embodiments, the prodrug of Dorzagliatin is one disclosed in WO 2023/040937A1, the disclosure of each of which is incorporated herein by reference in its entirety.

### Immediate-Release Formulation

In some embodiments, the pharmaceutical formulation is an immediate-release formulation.

In some embodiments, the pharmaceutical formulation further comprises one or more excipients, such as fillers, disintegrants, surfactants, glidants, lubricants. In some embodiments, the excipients are selected from the group consisting of fillers, disintegrants, surfactants, glidants, lubricants.

In some embodiments, examples of surfactants that are suitable for use herein include, but are not limited to, salts of alkyl sulfate such as sodium lauryl sulfate, polysorbates such as polysorbate 20, polysorbate 80 etc.; polyoxylglicerides such as caprylocaprol polyoxylglycerides, lauroyl polyoxylglycerides, linoleoyl polyoxylglycerides, oleoyl polyoxylglycerides, stearoyl polyoxylglicerides, polyoxyl stearate; poloxamers such as poloxamer 188; sorbitan ester such as sorbitan monooleate, sorbitan trioleate, sorbitan monostearate, sorbitan monolaurate, etc.; polyoxyethylene alkyl ethers, benzalkonium chloride, and combinations thereof.

In some embodiments, examples of fillers that are suitable for use herein include, but are not limited to, celluloses such as silicified microcrystalline cellulose, microcrystalline cellulose, powder cellulose, etc.; starches such as corn starch, maize starch, potato starch, pregelatinized starch, dextrin, etc.; sugar such as lactose (e.g. lactose monohydrate, spray-dried lactose, lactose anhydrous), sucrose, fructose, compressible sugar, maltose, dextrose, dextrates, etc.; sugar alcohols such as mannitol, sorbitol, xylitol etc.; calcium phosphate, calcium carbonate, calcium sulfate, kaolin, magnesium oxide, and combinations thereof.

In some embodiments, examples of disintegrants that are suitable for use herein include, but are not limited to, starches such as corn starch, pregelatinized starch, sodium starch glycolate, etc.; celluloses such as microcrystalline cellulose, powdered cellulose, methylcellulose, low-substituted hydroxypropyl cellulose, croscarmellose sodium, sodium carboxymethyl cellulose, calcium carboxymethylcellulose, etc.; alginates such as sodium alginate, calcium alginate, etc.; ion exchange resins such as polacrilin potassium; magnesium aluminum silicate, crospovidone, chitosan hydrochloride, and combinations thereof.

In some embodiments, examples of glidants that are suitable for use herein include, but are not limited to, silicon dioxide, colloidal silicone dioxide, talc, magnesium oxide, magnesium silicate, magnesium trisilicate, powdered cellulose, tribasic calcium phosphate, and combinations thereof.

In some embodiments, examples of lubricants that are suitable for use herein include, but are not limited to, magnesium stearate, polyethylene glycol, talc, hydrogenated vegetable oil, stearic acid, myristic acid, palmitic acid, poloxamer, calcium stearate, zinc stearate, glyceryl behenate, glyceryl monostearate, sodium lauryl sulfate, sodium stearyl fumarate, and combinations thereof.

In some embodiments, the pharmaceutical formulation comprises the components with the following amounts (by weight):
about 5-90% of glucokinase activator, or a prodrug (e.g., the compound of formula (I), or a corresponding amount of the pharmaceutically acceptable salt thereof);
about 10-85% of filler(s);
about 0-25% of disintegrant(s);
about 0-15% of surfactant(s);
about 0-10% of glidant(s); and
about 0-15% of lubricant(s).

In some embodiments, the pharmaceutical formulation comprises the components with the following amounts (by weight):
about 5-60% of the compound of formula (I), or a corresponding amount of the pharmaceutically acceptable salt thereof (or other glucokinase activator, or a prodrug thereof);
about 30-85% of silicified microcrystalline cellulose (or other filler);
about 0-25% of croscarmellose sodium (or other disintegrant);
about 0-15% of sodium lauryl sulfate (or other surfactant);
about 0-10% of colloidal silicone dioxide (or other glidant); and
about 0-15% of magnesium stearate (or other lubricant).

In some embodiments, the pharmaceutical formulation comprises the components with the following amounts (by weight):

(a)
about 40-50% of the compound of formula (I), or a corresponding amount of the pharmaceutically acceptable salt thereof (or other glucokinase activator, or a prodrug thereof);
about 42-52% of silicified microcrystalline cellulose (or other filler);
about 0.5-10% of croscarmellose sodium (or other disintegrant);
about 1-8% of sodium lauryl sulfate (or other surfactant);
about 0.1-3% of colloidal silicone dioxide (or other glidant); and
about 0.2-5% of magnesium stearate (or other lubricant).

In some embodiments, the pharmaceutical formulation comprises the components with the following amounts (by weight):

(b)
about 30-37% of the compound of formula (I), or a corresponding amount of the pharmaceutically acceptable salt thereof (or other glucokinase activator, or a prodrug thereof);
about 55-70% of silicified microcrystalline cellulose (or other filler);
about 0.5-10% of croscarmellose sodium (or other disintegrant);
about 0-8% of sodium lauryl sulfate (or other surfactant);
about 0-3% of colloidal silicone dioxide (or other glidant); and
about 0-5% of magnesium stearate (or other lubricant).

In some embodiments, the pharmaceutical formulation comprises the components with the following amounts (by weight):

(c)
about 20-25% of the compound of formula (I), or a corresponding amount of the pharmaceutically acceptable salt thereof (or other glucokinase activator, or a prodrug thereof);
about 60-76% of silicified microcrystalline cellulose (or other filler);
about 0.5-10% of croscarmellose sodium (or other disintegrant);
about 1-8% of sodium lauryl sulfate (or other surfactant);
about 0.1-3% of colloidal silicone dioxide (or other glidant); and
about 0.2-5% of magnesium stearate (or other lubricant).

In some embodiments, the pharmaceutical formulation comprises the components with the following amounts (by weight):

(d)
about 10-12% of the compound of formula (I), or a corresponding amount of the pharmaceutically acceptable salt thereof (or other glucokinase activator, or a prodrug thereof);
about 70-90% (e.g., about 70-85%) of silicified microcrystalline cellulose (or other filler);
about 0.5-10% of croscarmellose sodium (or other disintegrant);
about 1-8% of sodium lauryl sulfate (or other surfactant);
about 0.1-3% of colloidal silicone dioxide (or other glidant); and
about 0.2-5% of magnesium stearate (or other lubricant).

In some embodiments, the pharmaceutical formulation comprises the components with the following amounts (by weight):

(e)
about 30-37% of the compound of formula (I), or a corresponding amount of the pharmaceutically acceptable salt thereof (or other glucokinase activator, or a prodrug thereof);
about 53-65% of silicified microcrystalline cellulose (or other filler);
about 0.5-10% of croscarmellose sodium (or other disintegrant);
about 1-8% of sodium lauryl sulfate (or other surfactant);
about 0-3% of colloidal silicone dioxide (or other glidant); and
about 0.2-5% of magnesium stearate (or other lubricant).

In some embodiments, the pharmaceutical formulation comprises the components with the following amounts (by weight):

(f)
about 30-37% of the compound of formula (I), or a corresponding amount of the pharmaceutically acceptable salt thereof (or other glucokinase activator, or a prodrug thereof);
about 46-57% of silicified microcrystalline cellulose (or other filler);
about 0.5-10% of croscarmellose sodium (or other disintegrant);
about 5-15% of sodium lauryl sulfate (or other surfactant);
about 0-3% of colloidal silicone dioxide (or other glidant); and
about 0.2-5% of magnesium stearate (or other lubricant).

In some embodiments, the pharmaceutical formulation comprises the components with the following amounts (by weight):

(g)
about 33-42% of the compound of formula (I), or a corresponding amount of the pharmaceutically acceptable salt thereof (or other glucokinase activator, or a prodrug thereof);
about 48-59% of silicified microcrystalline cellulose (or other filler);
about 0.5-10% of croscarmellose sodium (or other disintegrant);
about 0-8% of sodium lauryl sulfate (or other surfactant);
about 0.1-8% of colloidal silicone dioxide (or other glidant); and
about 0.2-5% of magnesium stearate (or other lubricant).

In some embodiments, the pharmaceutical formulation comprises the components with the following amounts (by weight):
about 5-60% of the compound of formula (I), or a corresponding amount of the pharmaceutically acceptable salt thereof (or other glucokinase activator, or a prodrug thereof);
about 30-85% of silicified microcrystalline cellulose (or other filler);
about 0-25% of croscarmellose sodium (or other disintegrant);
about 0-15% of sodium lauryl sulfate (or other surfactant);
about 0-10% of colloidal silicone dioxide (or other glidant); and
about 0-15% of magnesium stearate (or other lubricant).

In some embodiments, the pharmaceutical formulation comprises the components with the following amounts (by weight):

(h)
about 16-20% of the compound of formula (I), or a corresponding amount of the pharmaceutically acceptable salt thereof (or other glucokinase activator, or a prodrug thereof);
about 66-81% of silicified microcrystalline cellulose (or other filler);
about 2.5-3.5% of croscarmellose sodium (or other disintegrant);
about 34% of sodium lauryl sulfate (or other surfactant);
about 0.1-3% of colloidal silicone dioxide (or other glidant); and
about 0.2-5% of magnesium stearate (or other lubricant).

In some embodiments, the pharmaceutical formulation comprises the components with the following amounts (by weight):

(i)
about 32-40% of the compound of formula (I), or a corresponding amount of the pharmaceutically acceptable salt thereof (or other glucokinase activator, or a prodrug thereof);
about 49-60% of silicified microcrystalline cellulose (or other filler);
about 2.5-3.5% of croscarmellose sodium (or other disintegrant);
about 34% of sodium lauryl sulfate (or other surfactant);
about 0.1-3% of colloidal silicone dioxide (or other glidant); and
about 0.2-5% of magnesium stearate (or other lubricant).

In some embodiments, the pharmaceutical formulation comprises the components with the following amounts (by weight):

(j)
about 32-40% of the compound of formula (I), or a corresponding amount of the pharmaceutically acceptable salt thereof (or other glucokinase activator, or a prodrug thereof);
about 46-57% of silicified microcrystalline cellulose (or other filler);
about 2.5-3.5% of croscarmellose sodium (or other disintegrant);
about 6-8% of sodium lauryl sulfate (or other surfactant);
about 0.1-3% of colloidal silicone dioxide (or other glidant); and
about 0.2-5% of magnesium stearate (or other lubricant).

In some embodiments, the pharmaceutical formulation comprises the glucokinase activator, or the prodrug in a dose (alternatively, a unit dose) ranging from about 1 mg to about 1500 mg, from about 1.23 mg to about 1230 mg, from about 1 mg to about 1000 mg, from about 5 mg to about 615 mg, from about 10 mg to about 492 mg, from about 15 mg to about 369 mg, from about 20 mg to about 246 mg, from about 50 mg to about 184 mg, or from about 75 mg to about 123 mg.

In some embodiments, the pharmaceutical formulation comprises the glucokinase activator, or the prodrug in a dose (alternatively, a unit dose) of about 10 mg, about 12.3 mg, about 18.4 mg, about 20 mg, about 24.6 mg, about 25 mg, about 30 mg, about 30.75 mg, about 35 mg, about 36.9 mg, about 40 mg, about 43.05 mg, about 45 mg, about 49.2 mg, about 50 mg, about 55 mg, about 55.35 mg, about 60 mg, about 61.5 mg, about 65 mg, about 67.65 mg, about 70 mg, about 73.8 mg, about 75 mg, about 79.95 mg, about 80 mg, about 85 mg, about 86.1 mg, about 90 mg, about 92 mg, about 95 mg, about 98.4 mg, about 100 mg, about 104.55 mg, about 110 mg, about 110.7 mg, about 116.85 mg, about 120 mg, about 123 mg, about 130 mg, about 135.3 mg, about 140 mg, about 147.6 mg, about 150 mg, about 159.9 mg, about 160 mg, about 170 mg, about 172.2 mg, about 180 mg, or about 184 mg, about 190 mg, about 196.8 mg, about 200 mg, about 209.1 mg, about 221.4 mg, about 233.7 mg, about 246 mg, about 250 mg, about 300 mg, about 307.5 mg, about 350 mg, about 369 mg, about 400 mg, about 430.5 mg, or about 492 mg.

In some embodiments, the prodrug is the compound of formula (I), or a corresponding amount of the pharmaceutically acceptable salt thereof, and the compound of formula (I) is present in a dose (alternatively, a unit dose) ranging from about 1.23 mg to about 1230 mg, from about 6.15 mg to about 615 mg, from about 12.3 mg to about 492 mg, from about 18.45 mg to about 369 mg, from about 24.6 mg to about 246 mg, from about 30.75 mg to about 246 mg, from about 61.5 mg to about 184 mg, or from about 92 mg to about 123 mg. In some embodiments, the dose is from about 30.75 mg to about 61.5 mg, from about 61.5 mg to about 92 mg, or from about 92 mg to about 123 mg. In some embodiments, the dose is from about 61.5 mg to about 123 mg.

In some embodiments, the compound of formula (I) is present in a dose (alternatively, a unit dose) of about 10 mg, about 12.3 mg, 18.4 mg, about 20 mg, about 24.6 mg, about 25 mg, about 30 mg, about 30.75 mg, about 35 mg, about 36.9 mg, about 40 mg, about 43.05 mg, about 45 mg, about 49.2 mg, about 50 mg, about 55 mg, about 55.35 mg, about 60 mg, about 61.5 mg, about 65 mg, about 67.65 mg, about 70 mg, about 73.8 mg, about 75 mg, about 79.95 mg, about 80 mg, about 85 mg, about 86.1 mg, about 90 mg, about 92 mg, about 95 mg, about 98.4 mg, about 100 mg, about 104.55 mg, about 110 mg, about 110.7 mg, about 116.85 mg, about 120 mg, about 123 mg, about 130 mg, about 135.3 mg, about 140 mg, about 147.6 mg, about 150 mg, about 159.9 mg, about 160 mg, about 170 mg, about 172.2 mg, about 180 mg, about 184 mg, about 190 mg, about 196.8 mg, about 200 mg, about 209.1 mg, about 221.4 mg, about 233.7 mg, about 246 mg, about 307.5 mg, about 369 mg, about 430.5 mg, or about 492 mg.

In some embodiments, the compound of formula (I) is present in a dose (alternatively, a unit dose) of about 30.75 mg, about 61.5 mg, about 92 mg, about 123 mg, about 184 mg, about 246 mg, about 307.5 mg, about 369 mg, about 430.5 mg, or about 492 mg. In some embodiments, the dose is about 30.75 mg. In some embodiments, the dose is about 61.5 mg. In some embodiments, the dose is about 92 mg. In some embodiments, the dose is about 123 mg.

In some embodiments, the pharmaceutical formulation comprises:
about 20-160 mg of the compound of formula (I), or a corresponding amount of the pharmaceutically acceptable salt thereof (or other glucokinase activator, or a prodrug thereof);
about 90-290 mg of silicified microcrystalline cellulose (or other filler);
about 0-11 mg of croscarmellose sodium (or other disintegrant);
about 0-14 mg of sodium lauryl sulfate (or other surfactant);
about 0-2 mg of colloidal silicone dioxide (or other glidant); and
about 1-4 mg of magnesium stearate (or other lubricant).
   In some embodiments, the total weight of such pharmaceutical formulation is from about 220 mg to about 300 mg. In some embodiments, the total weight of such pharmaceutical formulation is from about 245 mg to about 275 mg.

In some embodiments, the pharmaceutical formulation comprises:

(a)
about 100-150 mg of the compound of formula (I), or a corresponding amount of the pharmaceutically acceptable salt thereof (or other glucokinase activator, or a prodrug thereof);
about 100-155 mg of silicified microcrystalline cellulose (or other filler);
about 6.5-10 mg of croscarmellose sodium (or other disintegrant);
about 8-12 mg of sodium lauryl sulfate (or other surfactant);
about 1.0-2.0 mg of colloidal silicone dioxide (or other glidant); and
about 2.0-3.0 mg of magnesium stearate (or other lubricant).
   In some embodiments, the total weight of such pharmaceutical formulation is about 275 mg.

In some embodiments, the pharmaceutical formulation comprises:

(b)
about 75-110 mg of the compound of formula (I) or a corresponding amount of the pharmaceutically acceptable salt thereof (or other glucokinase activator, or a prodrug thereof);
about 135-205 mg of silicified microcrystalline cellulose (or other filler);
about 6.5-10 mg of croscarmellose sodium (or other disintegrant); and
about 2.0-3.5 mg of magnesium stearate (or other surfactant).
   In some embodiments, the total weight of such pharmaceutical formulation is about 275 mg.

In some embodiments, the pharmaceutical formulation comprises:

(c)
about 50-75 mg of the compound of formula (I), or a corresponding amount of the pharmaceutically acceptable salt thereof (or other glucokinase activator, or a prodrug thereof);
about 150-230 mg of silicified microcrystalline cellulose (or other filler);
about 6.5-10 mg of croscarmellose sodium (or other disintegrant);
about 8-12 mg of sodium lauryl sulfate (or other surfactant);
about 1.0-2.0 mg of colloidal silicone dioxide (or other glidant); and
about 2.0-3.0 mg of magnesium stearate (or other lubricant).
   In some embodiments, the total weight of such pharmaceutical formulation is about 275 mg.

In some embodiments, the pharmaceutical formulation comprises:

(d)
about 25-40 mg of the compound of formula (I), or a corresponding amount of the pharmaceutically acceptable salt thereof (or other glucokinase activator, or a prodrug thereof);
about 175-270 mg of silicified microcrystalline cellulose (or other filler);
about 6.5-10 mg of croscarmellose sodium (or other disintegrant);
about 8-12 mg of sodium lauryl sulfate (or other surfactant);
about 1.0-2.0 mg of colloidal silicone dioxide (or other glidant); and
about 2.0-3.0 mg of magnesium stearate (or other lubricant).
   In some embodiments, the total weight of such pharmaceutical formulation is about 275 mg.

In some embodiments, the pharmaceutical formulation comprises:

(e)
about 75-110 mg of the compound of formula (I) or a corresponding amount of the pharmaceutically acceptable salt thereof (or other glucokinase activator, or a prodrug thereof);
about 125-195 mg of silicified microcrystalline cellulose (or other filler);
about 6.5-10 mg of croscarmellose sodium (or other disintegrant);
about 8-12 mg of sodium lauryl sulfate (or other surfactant); and
about 2.0-3.5 mg of magnesium stearate (or other surfactant).
   In some embodiments, the total weight of such pharmaceutical formulation is about 275 mg.

In some embodiments, the pharmaceutical formulation comprises:

(f)
about 75-110 mg of the compound of formula (I) or a corresponding amount of the pharmaceutically acceptable salt thereof (or other glucokinase activator, or a prodrug thereof);
about 110-170 mg of silicified microcrystalline cellulose (or other filler);
about 6.5-10 mg of croscarmellose sodium (or other disintegrant);
about 25-35 mg of sodium lauryl sulfate (or other surfactant); and
about 2.0-3.5 mg of magnesium stearate (or other surfactant).
   In some embodiments, the total weight of such pharmaceutical formulation is about 275 mg.

In some embodiments, the pharmaceutical formulation comprises:

(g)
about 75-110 mg of the compound of formula (I) or a corresponding amount of the pharmaceutically acceptable salt thereof (or other glucokinase activator, or a prodrug thereof);
about 105-155 mg of silicified microcrystalline cellulose (or other filler);
about 6.5-10 mg of croscarmellose sodium (or other disintegrant);
about 8-12 mg of colloidal silicone dioxide (or other glidant); and
about 2.0-3.5 mg of magnesium stearate (or other surfactant).
   In some embodiments, the total weight of such pharmaceutical formulation is about 245 mg.

In some embodiments, the pharmaceutical formulation comprises:
about 40-320 mg of the compound of formula (I), or a corresponding amount of the pharmaceutically acceptable salt thereof (or other glucokinase activator, or a prodrug thereof);
about 120-350 mg of silicified microcrystalline cellulose (or other filler);
about 5-15 mg of croscarmellose sodium (or other disintegrant);
about 5-30 mg of sodium lauryl sulfate (or other surfactant);
about 0-3 mg of colloidal silicone dioxide (or other glidant); and
about 3-10 mg of magnesium stearate (or other lubricant).
   In some embodiments, the total weight of such pharmaceutical formulation is from about 320 mg to about 360 mg. In some embodiments, the total weight of such pharmaceutical formulation is from about 337.5 mg to about 340 mg.

In some embodiments, the pharmaceutical formulation comprises:

(h)
about 48-75 mg of the compound of formula (I), or a corresponding amount of the pharmaceutically acceptable salt thereof (or other glucokinase activator, or a prodrug thereof);
about 195-300 mg of silicified microcrystalline cellulose (or other filler);
about 8-13 mg of croscarmellose sodium (or other disintegrant);
about 9-15 mg of sodium lauryl sulfate (or other surfactant);
about 1-3 mg of colloidal silicone dioxide (or other glidant); and
about 3-6 mg of magnesium stearate (or other lubricant).
   In some embodiments, the total weight of such pharmaceutical formulation is about 337.5 mg.

In some embodiments, the pharmaceutical formulation comprises:

(i)
about 100-150 mg of the compound of formula (I), or a corresponding amount of the pharmaceutically acceptable salt thereof (or other glucokinase activator, or a prodrug thereof);
about 145-220 mg of silicified microcrystalline cellulose (or other filler);
about 8-13 mg of croscarmellose sodium (or other disintegrant);
about 9-15 mg of sodium lauryl sulfate (or other surfactant);
about 1-3 mg of colloidal silicone dioxide (or other glidant); and
about 4-8 mg of magnesium stearate (or other lubricant).
   In some embodiments, the total weight of such pharmaceutical formulation is about 337.5 mg.

In some embodiments, the pharmaceutical formulation comprises:

(j)
about 100-150 mg of the compound of formula (I), or a corresponding amount of the pharmaceutically acceptable salt thereof (or other glucokinase activator, or a prodrug thereof);
about 130-210 mg of silicified microcrystalline cellulose (or other filler);
about 8-13 mg of croscarmellose sodium (or other disintegrant);
about 19-29 mg of sodium lauryl sulfate (or other surfactant);
about 1-3 mg of colloidal silicone dioxide (or other glidant); and
about 4-8 mg of magnesium stearate (or other lubricant).
   In some embodiments, the total weight of such pharmaceutical formulation is about 340 mg.

In some embodiments, the pharmaceutical formulation is IR1, which comprises the components with the following amounts (by weight):
about 44.73% of the compound of formula (I), or a corresponding amount of the pharmaceutically acceptable salt thereof;
about 47.09% of silicified microcrystalline cellulose;
about 3.09% of croscarmellose sodium;
about 3.64% of sodium lauryl sulfate;
about 0.54% of colloidal silicone dioxide; and
about 0.91% of magnesium stearate.

In some embodiments, the pharmaceutical formulation IR1 comprises:
about 123.0 mg of the compound of formula (I) or a corresponding amount of the pharmaceutically acceptable salt thereof;
about 129.5 mg of silicified microcrystalline cellulose;
about 8.5 mg of croscarmellose sodium;
about 10.0 mg of sodium lauryl sulfate;
about 1.5 mg of colloidal silicone dioxide; and
about 2.5 mg of magnesium stearate.

In some embodiments, the pharmaceutical formulation is IR2, which comprises the components with the following amounts (by weight):
about 33.5% of the compound of formula (I), or a corresponding amount of the pharmaceutically acceptable salt thereof;
about 62.4% of silicified microcrystalline cellulose;
about 3.1% of croscarmellose sodium;
about 0% of sodium lauryl sulfate;
about 0% of colloidal silicone dioxide; and
about 1.0% of magnesium stearate.

In some embodiments, the pharmaceutical formulation IR2 comprises:
about 92.0 mg of the compound of formula (I) or a corresponding amount of the pharmaceutically acceptable salt thereof;
about 171.7 mg of silicified microcrystalline cellulose;
about 8.5 mg of croscarmellose sodium; and
about 2.8 mg of magnesium stearate.

In some embodiments, the pharmaceutical formulation is IR3, which comprises the components with the following amounts (by weight):
about 33.5% of the compound of formula (I), or a corresponding amount of the pharmaceutically acceptable salt thereof;
about 58.8% of silicified microcrystalline cellulose;
about 3.1% of croscarmellose sodium;
about 3.6% of sodium lauryl sulfate;
about 0% of colloidal silicone dioxide; and
about 1.0% of magnesium stearate.

In some embodiments, the pharmaceutical formulation IR3 comprises:
about 92.0 mg of the compound of formula (I) or a corresponding amount of the pharmaceutically acceptable salt thereof;
about 161.7 mg of silicified microcrystalline cellulose;
about 8.5 mg of croscarmellose sodium;
about 10 mg of sodium lauryl sulfate; and
about 2.8 mg of magnesium stearate.

In some embodiments, the pharmaceutical formulation is IR4, which comprises the components with the following amounts (by weight):
about 33.5% of the compound of formula (I), or a corresponding amount of the pharmaceutically acceptable salt thereof;
about 51.5% of silicified microcrystalline cellulose;
about 3.1% of croscarmellose sodium;
about 10.9% of sodium lauryl sulfate;
about 0% of colloidal silicone dioxide; and
about 1.0% of magnesium stearate.

In some embodiments, the pharmaceutical formulation IR4 comprises:
about 92.0 mg of the compound of formula (I) or a corresponding amount of the pharmaceutically acceptable salt thereof;
about 141.7 mg of silicified microcrystalline cellulose;
about 8.5 mg of croscarmellose sodium;
about 30 mg of sodium lauryl sulfate; and
about 2.8 mg of magnesium stearate.

In some embodiments, the pharmaceutical formulation is IR5, which comprises the components with the following amounts (by weight):
about 37.6% of the compound of formula (I), or a corresponding amount of the pharmaceutically acceptable salt thereof;
about 53.8% of silicified microcrystalline cellulose;
about 3.5% of croscarmellose sodium;
about 0% of sodium lauryl sulfate;
about 4.1% of colloidal silicone dioxide; and
about 1.1% of magnesium stearate.

In some embodiments, the pharmaceutical formulation IR5 comprises:
about 92.0 mg of the compound of formula (I), or a corresponding amount of the pharmaceutically acceptable salt thereof;
about 131.7 mg of silicified microcrystalline cellulose;
about 8.5 mg of croscarmellose sodium;
about 10.0 mg of colloidal silicone dioxide; and
about 2.8 mg of magnesium stearate.

In some embodiments, the pharmaceutical formulation is IR6, which comprises the components with the following amounts (by weight):
about 22.4% of the compound of formula (I), or a corresponding amount of the pharmaceutically acceptable salt thereof;
about 69.5% of silicified microcrystalline cellulose;
about 3.1% of croscarmellose sodium;
about 3.6% of sodium lauryl sulfate;
about 0.5% of colloidal silicone dioxide; and
about 0.9% of magnesium stearate.

In some embodiments, the pharmaceutical formulation IR6 comprises:
about 61.5 mg of the compound of formula (I), or a corresponding amount of the pharmaceutically acceptable salt thereof;
about 191.0 mg of silicified microcrystalline cellulose;
about 8.5 mg of croscarmellose sodium; and
about 10 mg of sodium lauryl sulfate;
about 1.5 mg of colloidal silicone dioxide; and
about 2.5 mg of magnesium stearate.

In some embodiments, the pharmaceutical formulation is IR7, which comprises the components with the following amounts (by weight):
about 11.2% of the compound of formula (I), or a corresponding amount of the pharmaceutically acceptable salt thereof;
about 80.6% of silicified microcrystalline cellulose;
about 3.1% of croscarmellose sodium;
about 3.6% of sodium lauryl sulfate;
about 0.5% of colloidal silicone dioxide; and
about 0.9% of magnesium stearate.

In some embodiments, the pharmaceutical formulation IR7 comprises:
about 30.75 mg of the compound of formula (I), or a corresponding amount of the pharmaceutically acceptable salt thereof;
about 221.75 mg of silicified microcrystalline cellulose;
about 8.5 mg of croscarmellose sodium; and
about 10 mg of sodium lauryl sulfate;
about 1.5 mg of colloidal silicone dioxide; and
about 2.5 mg of magnesium stearate.

In some embodiments, the pharmaceutical formulation is IR8, which comprises the components with the following amounts (by weight):
about 18.22% of the compound of formula (I), or a corresponding amount of the pharmaceutically acceptable salt thereof;
about 73.11% of silicified microcrystalline cellulose;
about 3.09% of croscarmellose sodium;
about 3.64% of sodium lauryl sulfate;
about 0.55% of colloidal silicone dioxide; and
about 1.39% of magnesium stearate.

In some embodiments, the pharmaceutical formulation IR8 comprises:
about 61.50 mg of the compound of formula (I), or a corresponding amount of the pharmaceutically acceptable salt thereof;
about 246.75 mg of silicified microcrystalline cellulose;
about 10.42 mg of croscarmellose sodium;
about 12.28 mg of sodium lauryl sulfate;
about 1.85 mg of colloidal silicone dioxide; and
about 4.70 mg of magnesium stearate.

In some embodiments, the pharmaceutical formulation is IR9, which comprises the components with the following amounts (by weight):
about 36.44% of the compound of formula (I), or a corresponding amount of the pharmaceutically acceptable salt thereof;
about 54.47% of silicified microcrystalline cellulose;
about 3.09% of croscarmellose sodium;
about 3.64% of sodium lauryl sulfate;
about 0.55% of colloidal silicone dioxide; and
about 1.81% of magnesium stearate.

In some embodiments, the pharmaceutical formulation IR9 comprises:
about 123.00 mg of the compound of formula (I), or a corresponding amount of the pharmaceutically acceptable salt thereof;
about 183.85 mg of silicified microcrystalline cellulose;
about 10.42 mg of croscarmellose sodium;
about 12.28 mg of sodium lauryl sulfate;
about 1.85 mg of colloidal silicone dioxide; and
about 6.10 mg of magnesium stearate.

In some embodiments, the pharmaceutical formulation is IR10, which comprises the components with the following amounts (by weight):
about 36.18% of the compound of formula (I), or a corresponding amount of the pharmaceutically acceptable salt thereof;
about 51.38% of silicified microcrystalline cellulose;
about 3.09% of croscarmellose sodium;
about 7.06% of sodium lauryl sulfate;
about 0.53% of colloidal silicone dioxide; and
about 1.76% of magnesium stearate.

In some embodiments, the pharmaceutical formulation IR10 comprises:
about 123.00 mg of the compound of formula (I), or a corresponding amount of the pharmaceutically acceptable salt thereof;
about 174.70 mg of silicified microcrystalline cellulose;
about 10.50 mg of croscarmellose sodium;
about 24.00 mg of sodium lauryl sulfate;
about 1.80 mg of colloidal silicone dioxide; and
about 6.00 mg of magnesium stearate.

In some embodiments, the formulation is an oral solid formulation.

In some embodiments, the pharmaceutical formulation is present in the form of a tablet, a granule, a pill, or a capsule, alternatively is a tablet.

In some embodiments, the pharmaceutical formulation is film-coated tablets.

In some embodiments, the film-coated pharmaceutical formulation comprises coating agents selected from the group consisting of sodium carboxymethylcellulose, cellulose acetate, cellulose acetate phthalate, ethylcellulose, gelatin, pharmaceutical glaze, hydroxypropyl cellulose, hydroxypropylmethyl cellulose, hydroxypropyl methylcellulose phthalate, methacrylic acid copolymer, methylcellulose, polyethylene glycol, polyvinyl acetate phthalate, shellac, sucrose, titanium dioxide, carnauba wax, microcrystalline wax, zein, Polyvinyl Alcohol-Part, Hydrolyzed, Xanthan Gum, Gum Acacia, Macrogol (PEG) Polyvinyl Alcohol Graft Copolymer and Opadry.

In some embodiments, the coating agent is Opadry.

In some embodiments, the Opadry (such as Opadry II) is present in the amount of about 2-6% by weight of the core tablet.

In some embodiments, the immediate-release formulation has a dissolution rate of >60% within 15 min, 20 min, 30 min, 40 min, 45 min, 50 min or 60 min.

In some embodiments, the immediate-release formulation has a dissolution rate of >85% within 30 min, 40 min, 45 min, 50 min, 60 min.

In some embodiments, the pharmaceutical formulation is administered once-a-day.

In some embodiments, it is to provide an IR tablet (IR1) as described in Table 1. In some embodiments, it is to provide IR tablets (IR2-IR7) as described in Table 2. In some embodiments, it is to provide IR tablets (IR8-IR10) as described in Table 2-1.

In some embodiments, provided herein is a pharmaceutical formulation, which corresponds to the first part (alternatively, first layer, or immediate-release layer) of any two-parts (or bilayer) formulation provided herein, such as those described in the Bilayer Formulation section below and those described in the Examples. In other words, any such first part (alternatively, first layer, or immediate-release layer) is specifically provided herein as a standalone pharmaceutical formulation.

### Extended-Release Formulation

The present disclosure provides a pharmaceutical formulation, which is an extended-release formulation comprising: glucokinase activator, or a prodrug, or a pharmaceutically acceptable salt, an isotope labeled analogue, a crystalline form, a hydrate, a solvate, or a diastereomeric or enantiomeric form thereof, and release modifier(s).

In some embodiments, examples of release modifiers that are suitable for use herein include, but are not limited to, hydroxypropyl cellulose, hydroxypropyl methylcellulose ((HPMC, e.g. hypromellose E5, hypromellose K4M, hypromellose K100LV, hypromellose K100M), hydroxyethyl cellulose, hydroxyethyl methyl cellulose, methylcellulose, ethylcellulose, carboxymethylcellulose, polyvinylpyrrolidone, copovidone, polyethylene oxide (e.g. POLYOX™ N-60K, POLYOX™ N-80), polyethylene glycol, carbomer copolymer, carbomer homopolymer, carbomer interpolymer, polymethacrylates, starch such as corn starch, pregelatinized starch, etc.; shellac, guar gum, sodium alginate, xanthan gum, alginic acid, acacia, and combinations thereof.

In some embodiments, the release modifier is hypromellose E5, hypromellose K100LV, hypromellose K4M, or hypromellose K100M, or combinations thereof. In some embodiments, the release modifier is hypromellose E5, hypromellose K100LV, or hypromellose K100M, or combinations thereof.

In some embodiments, the release modifier is hypromellose K100M, which is a hypromellose having viscosity from about 75,000 to about 140,000 mPa·s, such as about 100,000 mPa·s (e.g. METHOCEL™ K100M Premium, 2208).

In some embodiments, the release modifier is hypromellose E5, which is a hypromellose having viscosity from about 1 to about 100 mPa·s, e.g., from about 1 to about 50 mPa·s, such as about 5 mPa·s (e.g. METHOCEL™ E5 Premium LV, 2910).

In some embodiments, the release modifier is hypromellose K100LV, which is a hypromellose having viscosity from about 1 to about 1000 mPa·s, e.g., from about 1 to about 500 mPa·s, such as about 100 mPa·s (e.g. METHOCEL™ K100 Premium LV, 2208).

In some embodiments, the release modifier is hypromellose K4M, which is a hypromellose having viscosity from about 100 to about 20,000 mPa·s, e.g., from about 1000 to about 10,000 mPa·s, such as about 4000 mPa·s.

Typical viscosity values for 2% (w/v) aqueous solutions of Methocel (Dow Wolff Cellulosics) were measured at 20° C.

In some embodiments, the release modifier is present in a dose (alternatively, a unit dose) ranging from about 10-80%, alternatively from about 15-45%, alternatively from about 20-40%, alternatively about 25-35%, such as about 30%, said weight percentage is based on the total weight of the formulation.

In some embodiments, the pharmaceutical formulation further comprising one or more excipients selected from the group consisting of fillers, surfactants, glidants, lubricants.

In some embodiments, the pharmaceutical formulation comprises the components with the following amounts (by weight):
about 5-90% (such as 30-60%) of glucokinase activator, or a prodrug (e.g., the compound of formula (I), or a pharmaceutically acceptable salt thereof);
about 5-85% (such as 5-65%) of filler(s);
about 10-80% (such as 15-45%) of release modifier(s);
about 0-40% (such as 0.1-40%) of surfactant(s);
about 0-10% (such as 0.1-10%) of glidant(s); and
about 0-15% (such as 0.1-5%) of lubricant(s).

In some embodiments, the pharmaceutical formulation is ER1, which comprises the components with the following amounts (by weight):
about 30-60% (such as 35-55%) of the compound of formula (I), or a corresponding amount of the pharmaceutically acceptable salt thereof;
about 5-65% (such as 5-15%) of silicified microcrystalline cellulose;
about 15-45% (such as 25-35%) of hypromellose100 LV;
about 10-40% (such as 13-17%) of sodium lauryl sulfate;
about 0.1-10% (such as 0.1-3%) of colloidal silicone dioxide; and
about 0.1-15% (such as 0.1-5%) of magnesium stearate.

alternatively, the pharmaceutical formulation ER1 comprises the components with the following amounts (by weight):
about 44.7% of the compound of formula (I), or a corresponding amount of the pharmaceutically acceptable salt thereof;
about 9.5% of silicified microcrystalline cellulose;
about 29.8% of hypromellose K100LV;
about 14.5% of sodium lauryl sulfate;
about 0.5% of colloidal silicone dioxide, and
about 0.9% of magnesium stearate.

In some embodiments, the pharmaceutical formulation ER1 comprises:
about 80-200 mg (such as 100-150 mg) of the compound of formula (I), or a corresponding amount of the pharmaceutically acceptable salt thereof,
about 1-180 mg (such as 20-30 mg) of silicified microcrystalline cellulose;
about 40-125 mg (such as 70-100 mg) hypromellose K100LV;
about 0-3 mg (such as 0.1-2 mg) colloidal silicone dioxide;
about 20-110 mg (such as 30-50 mg) of sodium lauryl sulfate; and
about 0-5 mg (such as 2-3 mg) of magnesium stearate;
   alternatively, the pharmaceutical formulation ER1 comprises:
about 123 mg of the compound of formula (I), or a corresponding amount of the pharmaceutically acceptable salt thereof;
about 26 mg of silicified microcrystalline cellulose;
about 82 mg of hypromellose K100LV;
about 1.5 mg of colloidal silicone dioxide;
about 40 mg of sodium lauryl sulfate; and
about 2.5 mg of magnesium stearate.

In some embodiments, the pharmaceutical formulation is ER2, which comprises the components with the following amounts (by weight):
about 30-60% (such as 35-55%) of the compound of formula (I), or a corresponding amount of the pharmaceutically acceptable salt thereof;
about 5-65% (such as 15-45%) of silicified microcrystalline cellulose;
about 15-45% (such as 25-35%) of hypromellose K100M;
about 0.1-20% (such as 3-4%) of sodium lauryl sulfate;
about 0.1-10% (such as 0.1-3%) of colloidal silicone dioxide; and
about 0.1-15% (such as 0.1-5%) of magnesium stearate.
   alternatively, the pharmaceutical formulation ER2 comprises the components with the following amounts (by weight):
about 44.73% of the compound of formula (I), or a corresponding amount of the pharmaceutically acceptable salt thereof;
about 20.36% of silicified microcrystalline cellulose;
about 29.82% of hypromellose K100M;
about 3.64% of sodium lauryl sulfate;
about 0.54% of colloidal silicone dioxide; and
about 0.91% of magnesium stearate.

In some embodiments, the pharmaceutical formulation ER2 comprises:
about 80-200 mg (such as 100-150 mg) of the compound of formula (I), or a corresponding amount of the pharmaceutically acceptable salt thereof,
about 1-180 mg (such as 20-90 mg) of silicified microcrystalline cellulose;
about 40-125 mg (such as 70-100 mg) hypromellose K100M;
about 0-3 mg (such as 0.1-2 mg) colloidal silicone dioxide;
about 5-50 mg (such as 5-20 mg) of sodium lauryl sulfate; and
about 0-5 mg (such as 2-3 mg) of magnesium stearate;
   alternatively, the pharmaceutical formulation ER2 comprises:
about 123 mg of the compound of formula (I), or a corresponding amount of the pharmaceutically acceptable salt thereof;
about 56 mg of silicified microcrystalline cellulose;
about 82 mg of hypromellose K100M;
about 1.5 mg of colloidal silicone dioxide;
about 10 mg of sodium lauryl sulfate; and
about 2.5 mg of magnesium stearate.

In some embodiments, the pharmaceutical formulation is ER3, which comprises the components with the following amounts (by weight):
about 30-60% (such as 35-55%) of the compound of formula (I), or a corresponding amount of the pharmaceutically acceptable salt thereof;
about 5-65% (such as 7-12%) of silicified microcrystalline cellulose;
about 15-45% (such as 25-35%) of hypromellose K100M;
about 0-10% (such as 0-3%) colloidal silicone dioxide;
about 10-40% (such as 13-17%) of sodium lauryl sulfate; and
about 0-15% (such as 0-5%) of magnesium stearate;
   alternatively, the pharmaceutical formulation ER3 comprises the components with the following amounts (by weight):
about 44.7% of the compound of formula (I), or a corresponding amount of the pharmaceutically acceptable salt thereof;
about 9.5% of silicified microcrystalline cellulose;
about 29.8% of hypromellose K100M;
about 0.5% of colloidal silicone dioxide;
about 14.5% of sodium lauryl sulfate; and
about 0.9% of magnesium stearate.

In some embodiments, the pharmaceutical formulation ER3 comprises:
about 80-200 mg (such as 100-150 mg) of the compound of formula (I), or a corresponding amount of the pharmaceutically acceptable salt thereof,
about 1-180 mg (such as 20-30 mg) of silicified microcrystalline cellulose;
about 40-125 mg (such as 70-100 mg) hypromellose K100M;
about 0-3 mg (such as 0.1-2 mg) colloidal silicone dioxide;
about 20-110 mg (such as 30-50 mg) of sodium lauryl sulfate; and
about 0-5 mg (such as 2-3 mg) of magnesium stearate;
   alternatively, the pharmaceutical formulation ER3 comprises:
about 123 mg of the compound of formula (I), or a corresponding amount of the pharmaceutically acceptable salt thereof;
about 26 mg of silicified microcrystalline cellulose;
about 82 mg of hypromellose K100M;
about 1.5 mg of colloidal silicone dioxide;
about 40 mg of sodium lauryl sulfate; and
about 2.5 mg of magnesium stearate.

In some embodiments, the pharmaceutical formulation is ER4, which comprises the components with the following amounts (by weight):
about 30-60% (such as 35-55%) of the compound of formula (I), or a corresponding amount of the pharmaceutically acceptable salt thereof;
about 5-65% (such as 7-12%) of silicified microcrystalline cellulose;
about 15-45% (such as 25-35%) of hypromellose E5;
about 0-10% (such as 0-3%) colloidal silicone dioxide;
about 10-40% (such as 13-17%) of sodium lauryl sulfate; and
about 0-15% (such as 0-5%) of magnesium stearate;
   alternatively, the pharmaceutical formulation ER4 comprises the components with the following amounts (by weight):
about 44.7% of the compound of formula (I), or a corresponding amount of the pharmaceutically acceptable salt thereof;
about 9.5% of silicified microcrystalline cellulose;
about 29.8% of hypromellose E5;
about 0.5% of colloidal silicone dioxide;
about 14.5% of sodium lauryl sulfate; and
about 0.9% of magnesium stearate.

In some embodiments, the pharmaceutical formulation ER4 comprises:
about 80-200 mg (such as 100-150 mg) of the compound of formula (I), or a corresponding amount of the pharmaceutically acceptable salt thereof,
about 1-180 mg (such as 20-30 mg) of silicified microcrystalline cellulose;
about 40-125 mg (such as 70-100 mg) hypromellose E5;
about 0-3 mg (such as 0.1-2 mg) colloidal silicone dioxide;
about 20-110 mg (such as 30-50 mg) of sodium lauryl sulfate; and
about 0-5 mg (such as 2-3 mg) of magnesium stearate;
   alternatively, the pharmaceutical formulation ER4 comprises:
about 123 mg of the compound of formula (I), or a corresponding amount of the pharmaceutically acceptable salt thereof;
about 26 mg of silicified microcrystalline cellulose;
about 82 mg of hypromellose E5;
about 1.5 mg of colloidal silicone dioxide;
about 40 mg of sodium lauryl sulfate; and
about 2.5 mg of magnesium stearate.

In some embodiments, the pharmaceutical formulation is ER5, which comprises the components with the following amounts (by weight):
about 30-60% (such as 35-55%) of the compound of formula (I), or a corresponding amount of the pharmaceutically acceptable salt thereof;
about 5-65% (such as 7-12%) of silicified microcrystalline cellulose;
about 10-22.5% (such as 13-17%) of hypromellose E5;
about 10-22.5% (such as 13-17%) of hypromellose K100LV;
about 0-10% (such as 0.1-3%) of colloidal silicone dioxide;
about 10-40% (such as 13-17%) of sodium lauryl sulfate; and
about 0-15% (such as 0.2-5%) of magnesium stearate;
   alternatively, the pharmaceutical formulation ER5 comprises the components with the following amounts (by weight):
about 44.7% of the compound of formula (I), or a corresponding amount of the pharmaceutically acceptable salt thereof;
about 9.5% of silicified microcrystalline cellulose;
about 14.9% of hypromellose E5;
about 14.9% of hypromellose K100LV;
about 0.5% of colloidal silicone dioxide;
about 14.5% of sodium lauryl sulfate;
about 0.9% of magnesium stearate.

In some embodiments, the pharmaceutical formulation ER5 comprises:
about 80-200 mg (such as 100-150 mg) of the compound of formula (I), or a corresponding amount of the pharmaceutically acceptable salt thereof,
about 1-180 mg (such as 20-30 mg) of silicified microcrystalline cellulose;
about 20-60 mg (such as 30-50 mg) of hypromellose E5;
about 20-60 mg (such as 30-50 mg) of hypromellose K100LV;
about 0-3 mg (such as 0.1-2 mg) of colloidal silicone dioxide;
about 20-110 mg (such as 30-50 mg) of sodium lauryl sulfate; and
about 0-5 mg (such as 2-3 mg) of magnesium stearate.
   alternatively, the pharmaceutical formulation ER5 comprises:
about 123 mg of the compound of formula (I), or a corresponding amount of the pharmaceutically acceptable salt thereof;
about 26 mg of silicified microcrystalline cellulose;
about 41 mg of hypromellose E5;
about 41 mg of hypromellose K100LV;
about 1.5 mg of colloidal silicone dioxide;
about 40 mg of sodium lauryl sulfate;
about 2.5 mg of magnesium stearate.

In some embodiments, the pharmaceutical formulation is ER6, which comprises the components with the following amounts (by weight):
about 30-60% (such as 35-55%) of the compound of formula (I), or a corresponding amount of the pharmaceutically acceptable salt thereof;
about 5-65% (such as 15-45%) of silicified microcrystalline cellulose;
about 15-45% (such as 25-35%) of hypromellose K100M;
about 0.1-10% (such as 0.1-3%) of colloidal silicone dioxide; and
about 0.1-15% (such as 0-5%) of magnesium stearate;
   alternatively, the pharmaceutical formulation ER6 comprises the components with the following amounts (by weight):
   about 44.7% of the compound of formula (I), or a corresponding amount of the pharmaceutically acceptable salt thereof, about 24% of silicified microcrystalline cellulose, about 29.8% of hypromellose K100M, about 0.5% of colloidal silicone dioxide, and about 0.9% of magnesium stearate.

In some embodiments, the pharmaceutical formulation ER6 comprises:
about 80-200 mg (such as 100-150 mg) of the compound of formula (I), or a corresponding amount of the pharmaceutically acceptable salt thereof,
about 1-180 mg (such as 20-100 mg) of silicified microcrystalline cellulose;
about 40-125 mg (such as 70-100 mg) hypromellose K100M;
about 0-3 mg (such as 0.1-2 mg) colloidal silicone dioxide; and
about 0-5 mg (such as 2-3 mg) of magnesium stearate;
   alternatively, the pharmaceutical formulation ER6 comprises:
about 123 mg of the compound of formula (I), or a corresponding amount of the pharmaceutically acceptable salt thereof;
about 66 mg of silicified microcrystalline cellulose;
about 82 mg of hypromellose K100M;
about 1.5 mg of colloidal silicone dioxide; and
about 2.5 mg of magnesium stearate.

In some embodiments, the pharmaceutical formulation comprises the components with the following amounts (by weight):
about 30-60% (such as 37-55%) of the compound of formula (I), or a corresponding amount of the pharmaceutically acceptable salt thereof;
about 0-65% (such as 5-15%) of filler(s);
about 15-45% (such as 25-35%) of release modifier(s);
about 0-40% (such as 12-17%, or 20-25%) of surfactant(s);
about 0-10% (such as 0.1-3%) of glidant(s); and
about 0-15% (such as 0-5%) of lubricant(s).

In some embodiments, the pharmaceutical formulation is ER7, which comprises the components with the following amounts (by weight):
about 30-60% (such as 37-55%) of the compound of formula (I), or a corresponding amount of the pharmaceutically acceptable salt thereof;
about 2-65% (such as 5-15%) of silicified microcrystalline cellulose;
about 15-45% (such as 25-35%) of hypromellose E5;
about 5-40% (such as 12-17%) of sodium lauryl sulfate; and
about 0-15% (such as 0-5%) of magnesium stearate;
   alternatively, the pharmaceutical formulation ER7 comprises the components with the following amounts (by weight):
about 46% of the compound of formula (I), or a corresponding amount of the pharmaceutically acceptable salt thereof;
about 8% of silicified microcrystalline cellulose;
about 30% of hypromellose E5;
about 15% of sodium lauryl sulfate; and
about 1% of magnesium stearate.

In some embodiments, the pharmaceutical formulation ER7 comprises:
about 120-250 mg (such as 150-220 mg) of the compound of formula (I), or a corresponding amount of the pharmaceutically acceptable salt thereof,
about 10-100 mg (such as 20-50 mg) of silicified microcrystalline cellulose;
about 60-180 mg (such as 100-140 mg) of hypromellose E5;
about 20-160 mg (such as 50-70 mg) of sodium lauryl sulfate;
about 1-60 mg (such as 3-7 mg) of magnesium stearate; and
   alternatively, the pharmaceutical formulation ER7 comprises:
about 184 mg of the compound of formula (I), or a corresponding amount of the pharmaceutically acceptable salt thereof;
about 32.0 mg of silicified microcrystalline cellulose;
about 120.0 mg of hypromellose E5;
about 60 mg of sodium lauryl sulfate; and
about 4.0 mg of magnesium stearate.

In some embodiments, the pharmaceutical formulation is ER8, which comprises the components with the following amounts (by weight):
about 30-60% (such as 37-55%) of the compound of formula (I), or a corresponding amount of the pharmaceutically acceptable salt thereof;
about 15-45% (such as 25-35%) of hypromellose E5;
about 10-40% (such as 20-25%) of sodium lauryl sulfate; and
about 0-15% (such as 0-5%) of magnesium stearate;
   alternatively, the pharmaceutical formulation ER8 comprises the components with the following amounts (by weight):
about 46% of the compound of formula (I), or a corresponding amount of the pharmaceutically acceptable salt thereof;
about 30% of hypromellose E5;
about 23% of sodium lauryl sulfate; and
about 1% of magnesium stearate.

In some embodiments, the pharmaceutical formulation ER8 comprises:
about 120-250 mg (such as 150-220 mg) of the compound of formula (I), or a corresponding amount of the pharmaceutically acceptable salt thereof,
about 60-180 mg (such as 100-140 mg) of hypromellose E5;
about 40-160 mg (such as 80-100 mg) of sodium lauryl sulfate; and
about 1-40 mg (such as 3-7 mg) of magnesium stearate;
   alternatively, the pharmaceutical formulation ER8 comprises:
about 184 mg of the compound of formula (I), or a corresponding amount of the pharmaceutically acceptable salt thereof;
about 120.0 mg of hypromellose E5;
about 92 mg of sodium lauryl sulfate;
about 4.0 mg of magnesium stearate.

In some embodiments, the pharmaceutical formulation is an oral solid formulation. In some embodiments, the pharmaceutical formulation is present in the form of a tablet, a granule, a pill, or a capsule, alternatively is a tablet.

In some embodiments, the pharmaceutical formulation is film-coated tablets. In some embodiments, the film-coated pharmaceutical formulation comprises coating agents selected from the group consisting of sodium carboxymethylcellulose, cellulose acetate, cellulose acetate phthalate, ethylcellulose, gelatin, pharmaceutical glaze, hydroxypropyl cellulose, hydroxypropylmethyl cellulose, hydroxypropyl methylcellulose phthalate, methacrylic acid copolymer, methylcellulose, polyethylene glycol, polyvinyl acetate phthalate, shellac, sucrose, titanium dioxide, carnauba wax, microcystalline wax, zein, Polyvinyl Alcohol-Part. Hydrolyzed, Xanthan Gum, Gum Acacia, Macrogol (PEG) Polyvinyl Alcohol Graft Copolymer and Opadry. In some embodiments, the coating agent is Opadry. In some embodiments, the Opadry (such as Opadry II) is present in the amount of about 2-6% by weight of the core tablet.

In an embodiment, it is to provide an ER tablet (ER1) as described in Table 3. In an embodiment, it is to provide an ER tablet (ER2) as described in Table 4. In an embodiment, it is to provide an ER tablet (ER3) as described in Table 5. In an embodiment, it is to provide an ER tablet (ER4-ER8) as described in Table 6. In an embodiment, it is to provide an ER tablet (ER9-ER12) as described in Table 6-1.

In some embodiments, the pharmaceutical formulation is administered once-a-day.

In some embodiments, provided herein is a pharmaceutical formulation, which corresponds to the second part (alternatively, second layer, or extended-release layer) of any two-parts (or bilayer) formulation provided herein, such as those described in the Bilayer Formulation section below and those described in the Examples. In other words, any such second part (alternatively, second layer, or extended-release layer) is specifically provided herein as a standalone pharmaceutical formulation.

### Bilayer Formulation

The present disclosure provides a pharmaceutical formulation, comprising at least two parts, wherein the first part is the immediate-release pharmaceutical formulation as defined in the context; and the second part is an extended-release formulation comprises: glucokinase activator, or a prodrug, or a pharmaceutically acceptable salt, an isotope labeled analogue, a crystalline form, a hydrate, a solvate, or a diastereomeric or enantiomeric form thereof; and release modifier(s).

In some embodiments, the glucokinase activator and the prodrug are as defined in the context.

In some embodiments, the extended-release formulation is as defined in the context.

In some embodiments, the release modifier is selected from the group consisting of hydroxypropyl cellulose, hydroxypropyl methylcellulose ((HPMC, e.g. hypromellose E5, hypromellose K4M, hypromellose K100LV, hypromellose K100M), hydroxyethyl cellulose, hydroxyethyl methyl cellulose, methylcellulose, ethylcellulose, carboxymethylcellulose, polyvinylpyrrolidone, copovidone, polyethylene oxide (e.g. POLYOX™ N-60K, POLYOX™ N-80), polyethylene glycol, carbomer copolymer, carbomer homopolymer, carbomer interpolymer, polymethacrylates, starch such as corn starch, pregelatinized starch, etc.; shellac, guar gum, sodium alginate, xanthan gum, alginic acid, acacia, or combinations thereof.

In some embodiments, the release modifier is hypromellose E5, hypromellose K100LV, hypromellose K4M, or hypromellose K100M, or combinations thereof. In some embodiments, the release modifier is hypromellose E5, hypromellose K100LV, or hypromellose K100M, or combinations thereof.

In some embodiments, the release modifier is hypromellose K100M, which is a hypromellose having viscosity from about 75,000 to about 140,000 mPa·s, such as about 100,000 mPa·s (e.g. METHOCEL™ K100M Premium, 2208).

In some embodiments, the release modifier is hypromellose E5, which is a hypromellose having viscosity from about 1 to about 100 mPa·s, e.g., from about 1 to about 50 mPa·s, such as about 5 mPa·s (e.g. METHOCEL™ E5 Premium LV, 2910).

In some embodiments, the release modifier is hypromellose K100LV, which is a hypromellose having viscosity from about 1 to about 1000 mPa·s, e.g., from about 1 to about 500 mPa·s, such as about 100 mPa·s (e.g. METHOCEL™ K100 Premium LV, 2208).

In some embodiments, the release modifier is hypromellose K4M, which is a hypromellose having viscosity from about 100 to about 20,000 mPa·s, e.g., from about 1000 to about 10,000 mPa·s, such as about 4000 mPa·s.

Typical viscosity values for 2% (w/v) aqueous solutions of Methocel (Dow Wolff Cellulosics) were measured at 20° C.

In some embodiments, the release modifier is present in a dose (alternatively, a unit dose) ranging from about 10-80%, alternatively from about 15-45%, alternatively from about 20-40%, alternatively about 25-35%, such as about 30%, said weight percentage is based on the total weight of the second part.

In some embodiments, the weight ratio of glucokinase activator, or the prodrug in the first part to the glucokinase activator, or the prodrug in the second part is about 1:10 to 10:1, about 1:9 to 9:1, about 1:4 to 4:1, about 3:7 to 7:3, about 2:3 to 3:2, about 3:4 to 4:3, about 4:5 to 5:4 or about 5:6 to 6:5; alternatively, the weight ratio of glucokinase activator in the first layer to the glucokinase activator in the second layer is about 1:1, about 1:2, about 2:3, about 3:4, about 4:5, about 5:6, about 2:1, about 3:2, about 4:3, about 5:4 or about 6:5. In one embodiment, the weight ratio of glucokinase activator in the first layer to the glucokinase activator in the second layer is about 2:3.

In some embodiments, the second part further comprises one or more excipients selected from the group consisting of fillers, surfactants, glidants, lubricants.

Examples of fillers, surfactants, glidants and lubricants are as described in the context.

In some embodiments, the second part comprises the components with the following amounts (by weight):
about 5-90% of glucokinase activator, or the prodrug (e.g., 5-60% the compound of formula (I), or a corresponding amount of the pharmaceutically acceptable salt thereof);
about 5-85% of filler(s);
about 10-80% of release modifier(s);
about 0-40% of surfactant(s);
about 0-10% of glidant(s); and
about 0-15% of lubricant(s).

In some embodiments, the second part comprises the components with the following amounts (by weight):
about 8-55% of the compound of formula (I), or a corresponding amount of the pharmaceutically acceptable salt thereof;
about 5-65% of filler(s);
about 15-45% of release modifier(s);
about 1-30% of surfactant(s);
about 0.1-5% of glidant(s); and
about 0.1-10% of lubricant(s).

In some embodiments, the second part comprises the components with the following amounts (by weight):
about 40-50% of the compound of formula (I), or a corresponding amount of the pharmaceutically acceptable salt thereof;
about 5-30% of filler(s);
about 25-35% of release modifier(s);
about 1-25% of surfactant(s);
about 0.1-3% of glidant(s); and
about 0.2-5% of lubricant(s).

In some embodiments, the second part comprises the components with the following amounts (by weight):
about 20-25% of the compound of formula (I), or a pharmaceutically acceptable salt thereof;
about 28-35% of filler(s);
about 24-33% of release modifier(s);
about 13-16% of surfactant(s);
about 0.1-3% of glidant(s); and
about 0.2-5% of lubricant(s).

In some embodiments, the second part comprises the components with the following amounts (by weight):
about 40-50% of the compound of formula (I), or a pharmaceutically acceptable salt thereof;
about 8-10% of filler(s);
about 24-33% of release modifier(s);
about 13-16% of surfactant(s);
about 0.1-3% of glidant(s); and
about 0.2-5% of lubricant(s).

In some embodiments, the second part comprises the components with the following amounts (by weight):
about 32-40% of the compound of formula (I), or a pharmaceutically acceptable salt thereof;
about 7-10% of filler(s);
about 26-33% of release modifier(s);
about 21-26% of surfactant(s);
about 0.1-3% of glidant(s); and
about 0.2-5% of lubricant(s).

In some embodiments, the first part comprising the components with the following amounts (by weight):
about 5-60% of the compound of formula (I), or a corresponding amount of the pharmaceutically acceptable salt thereof;
about 30-85% of filler(s);
about 0.5-25% of disintegrant(s);
about 0-15% of surfactant(s);
about 0-10% of glidant(s); and
about 0-15% of lubricant(s);
   said percentage based on the total weight of the first part;
   the second part comprising the components with the following amounts (by weight):
about 8-55% of the compound of formula (I), or a corresponding amount of the pharmaceutically acceptable salt thereof;
about 5-65% of filler(s);
about 15-45% of release modifier(s);
about 1-30% of surfactant(s);
about 0.1-3% of glidant(s); and
about 0.1-10% of lubricant(s);
   said percentage based on the total weight of the second part.

In some embodiments, the pharmaceutical formulation is present in the form of a tablet, a granule, a pill, or a capsule.

In some embodiments, the pharmaceutical formulation is a bilayer tablet.

In some embodiments, the pharmaceutical formulation is a bilayer tablet, and wherein, the first part is an immediate-release layer, comprising the components with the following amounts (by weight):
about 5-60% of the compound of formula (I), or a corresponding amount of the pharmaceutically acceptable salt thereof (or other glucokinase activator, or a prodrug thereof);
about 30-85% of silicified microcrystalline cellulose (or other filler);
about 0-25% (e.g., about 0.5-25%) of croscarmellose sodium (or other disintegrant);
about 0-15% of sodium lauryl sulfate (or other surfactant);
about 0-10% of colloidal silicone dioxide (or other glidant); and
about 0-15% of magnesium stearate (or other lubricant);
   said percentage based on the total weight of the immediate-release layer;
   the second part is an extended-release layer, comprising the components with the following amounts (by weight):
about 8-55% of the compound of formula (I), or a corresponding amount of the pharmaceutically acceptable salt thereof (or other glucokinase activator, or a prodrug thereof);
about 5-65% of silicified microcrystalline cellulose (or other filler);
about 15-45% of hypromellose E5 (or other release modifier);
about 1-30% of sodium lauryl sulfate (or other surfactant);
about 0.1-3% of colloidal silicone dioxide (or other glidant); and
about 0.1-10% of magnesium stearate (or other lubricant); or
about 8-55% of the compound of formula (I), or a corresponding amount of the pharmaceutically acceptable salt thereof (or other glucokinase activator, or a prodrug thereof);
about 5-65% of silicified microcrystalline cellulose (or other filler);
about 10-20% of hypromellose E5 (or other release modifier);
about 10-20% hypromellose 100LV (or other release modifier);
about 1-30% of sodium lauryl sulfate (or other surfactant);
about 0.1-3% of colloidal silicone dioxide (or other glidant); and
about 0.1-10% of magnesium stearate (or other lubricant); or
about 8-55% of the compound of formula (I), or a corresponding amount of the pharmaceutically acceptable salt thereof (or other glucokinase activator, or a prodrug thereof);
about 5-65% of silicified microcrystalline cellulose (or other filler);
about 15-45% of hypromellose K100LV (or other release modifier);
about 1-30% of sodium lauryl sulfate (or other surfactant);
about 0.1-3% of colloidal silicone dioxide (or other glidant); and
about 0.1-10% of magnesium stearate (or other lubricant); or
about 8-55% of the compound of formula (I), or a corresponding amount of the pharmaceutically acceptable salt thereof (or other glucokinase activator, or a prodrug thereof);
about 5-65% of silicified microcrystalline cellulose (or other filler);
about 15-45% of hypromellose K100M (or other release modifier);
about 0-30% of sodium lauryl sulfate (or other surfactant);
about 0.1-3% of colloidal silicone dioxide (or other glidant); and
about 0.1-10% of magnesium stearate (or other lubricant);
   said percentage based on the total weight of the extended-release layer; or
about 8-55% of the compound of formula (I), or a pharmaceutically acceptable salt thereof (or other glucokinase activator, or a prodrug thereof);
about 5-65% of silicified microcrystalline cellulose (or other filler);
about 15-45% of hypromellose K4M (or other release modifier);
about 1-30% of sodium lauryl sulfate (or other surfactant);
about 0.1-3% of colloidal silicone dioxide (or other glidant); and
about 0.1-10% of magnesium stearate (or other lubricant);
   said percentage based on the total weight of the extended-release layer.

In some embodiments, the pharmaceutical formulation is a bilayer tablet, and wherein, the first layer is an immediate-release layer, comprising the components with the following amounts (by weight):

(a)
about 40-50% of the compound of formula (I), or a corresponding amount of the pharmaceutically acceptable salt thereof (or other glucokinase activator, or a prodrug thereof);
about 42-52% of silicified microcrystalline cellulose (or other filler);
about 0.5-10% of croscarmellose sodium (or other disintegrant);
about 1-8% of sodium lauryl sulfate (or other surfactant);
about 0.1-3% of colloidal silicone dioxide (or other glidant); and
about 0.2-5% of magnesium stearate (or other lubricant);
   said percentage based on the total weight of the immediate-release layer.

In some embodiments, the pharmaceutical formulation is a bilayer tablet, and wherein, the first layer is an immediate-release layer, comprising the components with the following amounts (by weight):

(b)
about 30-37% of the compound of formula (I), or a corresponding amount of the pharmaceutically acceptable salt thereof (or other glucokinase activator, or a prodrug thereof);
about 55-70% of silicified microcrystalline cellulose (or other filler);
about 0.5-10% of croscarmellose sodium (or other disintegrant);
about 0-8% of sodium lauryl sulfate (or other surfactant);
about 0-3% of colloidal silicone dioxide (or other glidant); and
about 0-5% of magnesium stearate (or other lubricant);
   said percentage based on the total weight of the immediate-release layer.

In some embodiments, the pharmaceutical formulation is a bilayer tablet, and wherein, the first layer is an immediate-release layer, comprising the components with the following amounts (by weight):

(c)
about 20-25% of the compound of formula (I), or a corresponding amount of the pharmaceutically acceptable salt thereof (or other glucokinase activator, or a prodrug thereof);
about 60-76% of silicified microcrystalline cellulose (or other filler);
about 0.5-10% of croscarmellose sodium (or other disintegrant);
about 1-8% of sodium lauryl sulfate (or other surfactant);
about 0.1-3% of colloidal silicone dioxide (or other glidant); and
about 0.2-5% of magnesium stearate (or other lubricant);
   said percentage based on the total weight of the immediate-release layer.

In some embodiments, the pharmaceutical formulation is a bilayer tablet, and wherein, the first layer is an immediate-release layer, comprising the components with the following amounts (by weight):

(d)
about 10-12% of the compound of formula (I), or a corresponding amount of the pharmaceutically acceptable salt thereof (or other glucokinase activator, or a prodrug thereof);
about 70-85% of silicified microcrystalline cellulose (or other filler);
about 0.5-10% of croscarmellose sodium (or other disintegrant);
about 1-8% of sodium lauryl sulfate (or other surfactant);
about 0.1-3% of colloidal silicone dioxide (or other glidant); and
about 0.2-5% of magnesium stearate (or other lubricant);
   said percentage based on the total weight of the immediate-release layer.

In some embodiments, the pharmaceutical formulation is a bilayer tablet, and the second layer is an extended-release layer, comprising the components with the following amounts (by weight):
(A) about 40-50% of the compound of formula (I), or a corresponding amount of the pharmaceutically acceptable salt thereof (or other glucokinase activator, or a prodrug thereof); about 8.5-10% of silicified microcrystalline cellulose (or other filler); about 25-35% of hypromellose K100M (or other release modifier); about 13-16% of sodium lauryl sulfate (or other surfactant); about 0.1-3% of colloidal silicone dioxide (or other glidant); and about 0.2-5% of magnesium stearate (or other lubricant); said percentage based on the total weight of the second layer.

In some embodiments, the pharmaceutical formulation is a bilayer tablet, and the second layer is an extended-release layer, comprising the components with the following amounts (by weight):
(B) about 40-50% of the compound of formula (I), or a corresponding amount of the pharmaceutically acceptable salt thereof (or other glucokinase activator, or a prodrug thereof); about 18-22% of silicified microcrystalline cellulose (or other filler); about 25-35% of hypromellose K100M (or other release modifier); about 3-4% of sodium lauryl sulfate (or other surfactant); about 0.1-3% of colloidal silicone dioxide (or other glidant); and about 0.2-5% of magnesium stearate (or other lubricant), said percentage based on the total weight of the extended-release layer.

In some embodiments, the pharmaceutical formulation is a bilayer tablet, and the second layer is an extended-release layer, comprising the components with the following amounts (by weight):
(C) about 40-50% of the compound of formula (I), or a corresponding amount of the pharmaceutically acceptable salt thereof (or other glucokinase activator, or a prodrug thereof); about 8.5-10% of silicified microcrystalline cellulose (or other filler); about 25-35% of hypromellose E5 (or other release modifier); about 13-16% of sodium lauryl sulfate (or other surfactant); about 0.1-3% of colloidal silicone dioxide (or other glidant); and about 0.2-5% of magnesium stearate (or other lubricant), said percentage based on the total weight of the extended-release layer.

In some embodiments, the pharmaceutical formulation is a bilayer tablet, and the second layer is an extended-release layer, comprising the components with the following amounts (by weight):
(D) about 40-50% of the compound of formula (I), or a corresponding amount of the pharmaceutically acceptable salt thereof (or other glucokinase activator, or a prodrug thereof); about 8.5-10% of silicified microcrystalline cellulose (or other filler); about 13-17% of hypromellose E5 (or other release modifier); about 13-17% of hypromellose K100LV (or other release modifier); about 13-16% of sodium lauryl sulfate (or other surfactant); about 0.1-3% of colloidal silicone dioxide (or other glidant); and about 0.2-5% of magnesium stearate (or other lubricant), said percentage based on the total weight of the extended-release layer.

In some embodiments, the pharmaceutical formulation is a bilayer tablet, and the second layer is an extended-release layer, comprising the components with the following amounts (by weight):
(E) about 40-50% of the compound of formula (I), or a corresponding amount of the pharmaceutically acceptable salt thereof (or other glucokinase activator, or a prodrug thereof); about 8.5-10% of silicified microcrystalline cellulose (or other filler); about 25-35% of hypromellose K100LV (or other release modifier); about 13-16% of sodium lauryl sulfate (or other surfactant); about 0.1-3% of colloidal silicone dioxide (or other glidant); and about 0.2-5% of magnesium stearate (or other lubricant), said percentage based on the total weight of the extended-release layer.

In some embodiments, the pharmaceutical formulation is a bilayer tablet, and the second layer is an extended-release layer, comprising the components with the following amounts (by weight):
(F) about 40-50% of the compound of formula (I), or a corresponding amount of the pharmaceutically acceptable salt thereof (or other glucokinase activator, or a prodrug thereof); about 21-27% of silicified microcrystalline cellulose; about 25-35% of hypromellose K100M (or other release modifier); about 0.1-3% of colloidal silicone dioxide (or other glidant); and about 0.2-5% of magnesium stearate (or other lubricant), said percentage based on the total weight of the extended-release layer.

In some embodiments, the pharmaceutical formulation is a bilayer tablet, and the first part is an immediate-release layer, comprising the components with the following amounts (by weight):
about 40-50% of the compound of formula (I), or a corresponding amount of the pharmaceutically acceptable salt thereof (or other glucokinase activator, or a prodrug thereof);
about 42-52% of silicified microcrystalline cellulose (or other filler);
about 0.5-10% of croscarmellose sodium (or other disintegrant);
about 1-8% of sodium lauryl sulfate (or other surfactant);
about 0.1-3% of colloidal silicone dioxide (or other glidant); and
about 0.2-5% of magnesium stearate (or other lubricant);
   said percentage based on the total weight of the immediate-release layer;
   the second part is an extended-release layer, comprising the components with the following amounts (by weight):
about 40-50% of the compound of formula (I), or a corresponding amount of the pharmaceutically acceptable salt thereof (or other glucokinase activator, or a prodrug thereof);
about 8.5-10% of silicified microcrystalline cellulose (or other filler);
about 25-35% of hypromellose K100M (or other release modifier);
about 13-16% of sodium lauryl sulfate (or other surfactant);
about 0.1-3% of colloidal silicone dioxide (or other glidant); and
about 0.2-5% of magnesium stearate (or other lubricant), said percentage based on the total weight of the second layer.

In some embodiments, the pharmaceutical formulation is a bilayer tablet, and the first part is an immediate-release layer, comprising the components with the following amounts (by weight):
about 40-50% of the compound of formula (I), or a corresponding amount of the pharmaceutically acceptable salt thereof (or other glucokinase activator, or a prodrug thereof);
about 42-52% of silicified microcrystalline cellulose (or other filler);
about 0.5-10% of croscarmellose sodium (or other disintegrant);
about 1-8% of sodium lauryl sulfate (or other surfactant);
about 0.1-3% of colloidal silicone dioxide (or other glidant); and
about 0.2-5% of magnesium stearate (or other lubricant);
   said percentage based on the total weight of the immediate-release layer;
   the second part is an extended-release layer, comprising the components with the following amounts (by weight):
about 40-50% of the compound of formula (I), or a corresponding amount of the pharmaceutically acceptable salt thereof (or other glucokinase activator, or a prodrug thereof);
about 18-22% of silicified microcrystalline cellulose (or other filler);
about 25-35% of hypromellose K100M (or other release modifier);
about 3-4% of sodium lauryl sulfate (or other surfactant);
about 0.1-3% of colloidal silicone dioxide (or other glidant); and
about 0.2-5% of magnesium stearate (or other lubricant);
   said percentage based on the total weight of the extended-release layer.

In some embodiments, the pharmaceutical formulation is a bilayer tablet, and the first part is an immediate-release layer, comprising the components with the following amounts (by weight):
about 18-27% of the compound of formula (I), or a corresponding amount of the pharmaceutically acceptable salt thereof (or other glucokinase activator, or a prodrug thereof);
about 56-83% of silicified microcrystalline cellulose (or other filler);
about 0.5-10% of croscarmellose sodium (or other disintegrant);
about 1-8% of sodium lauryl sulfate (or other surfactant);
about 0.1-3% of colloidal silicone dioxide (or other glidant); and
about 0.2-5% of magnesium stearate (or other lubricant);
   said percentage based on the total weight of the immediate-release layer;
   the second part is an extended-release layer, comprising the components with the following amounts (by weight):
about 18-27% of the compound of formula (I), or a corresponding amount of the pharmaceutically acceptable salt thereof (or other glucokinase activator, or a prodrug thereof);
about 25-38% of silicified microcrystalline cellulose (or other filler);
about 25-35% of hypromellose K100M (or other release modifier);
about 13-16% of sodium lauryl sulfate (or other surfactant);
about 0.1-3% of colloidal silicone dioxide (or other glidant); and
about 0.2-5% of magnesium stearate (or other lubricant);
   said percentage based on the total weight of the second layer.

In some embodiments, the immediate-release layer comprising:
(a) about 44.73% of the compound of formula (I), or a corresponding amount of the pharmaceutically acceptable salt thereof; about 47.09% of silicified microcrystalline cellulose; about 3.09% of croscarmellose sodium; about 3.64% of sodium lauryl sulfate; about 0.54% of colloidal silicone dioxide; and about 0.91% of magnesium stearate; said percentage based on the total weight of the immediate-release layer.

In some embodiments, the immediate-release layer comprising:
(b) about 33.5% of the compound of formula (I), or a corresponding amount of the pharmaceutically acceptable salt thereof; about 62.4% of silicified microcrystalline cellulose; about 3.1% of croscarmellose sodium; and about 1.0% of magnesium stearate; said percentage based on the total weight of the immediate-release layer.

In some embodiments, the immediate-release layer comprising:
(c) about 22.4% of the compound of formula (I), or a corresponding amount of the pharmaceutically acceptable salt thereof; about 69.5% of silicified microcrystalline cellulose; about 3.1% of croscarmellose sodium; about 3.6% of sodium lauryl sulfate; about 0.5% of colloidal silicone dioxide; and about 0.9% of magnesium stearate; said percentage based on the total weight of the immediate-release layer.

In some embodiments, the immediate-release layer comprising:
(d) about 11.2% of the compound of formula (I), or a corresponding amount of the pharmaceutically acceptable salt thereof; about 80.6% of silicified microcrystalline cellulose; about 3.1% of croscarmellose sodium; about 3.6% of sodium lauryl sulfate; about 0.5% of colloidal silicone dioxide; and about 0.9% of magnesium stearate; said percentage based on the total weight of the immediate-release layer.

In some embodiments, the extended-release layer comprising:
(A) about 44.73% of the compound of formula (I), or a corresponding amount of the pharmaceutically acceptable salt thereof; about 9.45% of silicified microcrystalline cellulose; about 29.82% of hypromellose K100M; about 14.55% of sodium lauryl sulfate; about 0.54% of colloidal silicone dioxide; and about 0.91% of magnesium stearate; said percentage based on the total weight of the extended-release layer.

In some embodiments, the extended-release layer comprising:
(B) about 44.73% of the compound of formula (I), or a corresponding amount of the pharmaceutically acceptable salt thereof; about 20.36% of silicified microcrystalline cellulose; about 29.82% of hypromellose K100M; about 3.64% of sodium lauryl sulfate; about 0.54% of colloidal silicone dioxide; and about 0.91% of magnesium stearate; said percentage based on the total weight of the extended-release layer.

In some embodiments, the extended-release layer comprising:
(C) about 44.7% of the compound of formula (I), or a corresponding amount of the pharmaceutically acceptable salt thereof; about 9.5% of silicified microcrystalline cellulose; about 29.8% of hypromellose E5; about 14.5% of sodium lauryl sulfate; about 0.5% of colloidal silicone dioxide; and about 0.9% of magnesium stearate; said percentage based on the total weight of the extended-release layer.

In some embodiments, the extended-release layer comprising:
(D) about 44.7% of the compound of formula (I), or a corresponding amount of the pharmaceutically acceptable salt thereof; about 9.5% of silicified microcrystalline cellulose; about 14.9% of hypromellose E5; about 14.9% of hypromellose K100LV; about 14.5% of sodium lauryl sulfate; about 0.5% of colloidal silicone dioxide; and about 0.9% of magnesium stearate; said percentage based on the total weight of the extended-release layer.

In some embodiments, the extended-release layer comprising:
(E) about 44.7% of the compound of formula (I), or a corresponding amount of the pharmaceutically acceptable salt thereof; about 9.5% of silicified microcrystalline cellulose; about 29.8% of hypromellose K100LV; about 14.5% of sodium lauryl sulfate; about 0.5% of colloidal silicone dioxide; and about 0.9% of magnesium stearate; said percentage based on the total weight of the extended-release layer.

In some embodiments, the extended-release layer comprising:
(F) about 44.7% of the compound of formula (I), or a corresponding amount of the pharmaceutically acceptable salt thereof; about 24% of silicified microcrystalline cellulose; about 29.8% of hypromellose K100M; about 1.5% of colloidal silicone dioxide; and about 2.5% of magnesium stearate; said percentage based on the total weight of the extended-release layer.

In some embodiments, the pharmaceutical formulation is a bilayer tablet, and wherein, the first layer is an immediate-release layer, comprising the components with the following amounts (by weight):
(h)
about 16-20% of the compound of formula (I), or a corresponding amount of the pharmaceutically acceptable salt thereof (or other glucokinase activator, or a prodrug thereof);
about 66-81% of silicified microcrystalline cellulose (or other filler);
about 2.5-3.5% of croscarmellose sodium (or other disintegrant);
about 3-4% of sodium lauryl sulfate (or other surfactant);
about 0.1-3% of colloidal silicone dioxide (or other glidant); and
about 0.2-5% of magnesium stearate (or other lubricant);
   said percentage based on the total weight of the immediate-release layer.

In some embodiments, the pharmaceutical formulation is a bilayer tablet, and wherein, the first layer is an immediate-release layer, comprising the components with the following amounts (by weight):

(i)
about 32-40% of the compound of formula (I), or a corresponding amount of the pharmaceutically acceptable salt thereof (or other glucokinase activator, or a prodrug thereof);
about 49-60% of silicified microcrystalline cellulose (or other filler);
about 2.5-3.5% of croscarmellose sodium (or other disintegrant);
about 3-4% of sodium lauryl sulfate (or other surfactant);
about 0.1-3% of colloidal silicone dioxide (or other glidant); and
about 0.2-5% of magnesium stearate (or other lubricant);
   said percentage based on the total weight of the immediate-release layer.

In some embodiments, the pharmaceutical formulation is a bilayer tablet, and wherein, the first layer is an immediate-release layer, comprising the components with the following amounts (by weight):

(j)
about 32-40% of the compound of formula (I), or a corresponding amount of the pharmaceutically acceptable salt thereof (or other glucokinase activator, or a prodrug thereof);
about 46-57% of silicified microcrystalline cellulose (or other filler);
about 2.5-3.5% of croscarmellose sodium (or other disintegrant);
about 6-8% of sodium lauryl sulfate (or other surfactant);
about 0.1-3% of colloidal silicone dioxide (or other glidant); and
about 0.2-5% of magnesium stearate (or other lubricant);
   said percentage based on the total weight of the immediate-release layer.

In some embodiments, the pharmaceutical formulation is a bilayer tablet, and wherein, the first layer is an immediate-release layer, comprising the components with the following amounts (by weight):

(i-f)
about 18-27% of the compound of formula (I), or a corresponding amount of the pharmaceutically acceptable salt thereof (or other glucokinase activator, or a prodrug thereof);
about 56-83% of silicified microcrystalline cellulose (or other filler);
about 0.5-10% of croscarmellose sodium (or other disintegrant);
about 1-8% of sodium lauryl sulfate (or other surfactant);
about 0.1-3% of colloidal silicone dioxide (or other glidant); and
about 0.2-5% of magnesium stearate (or other lubricant);
   said percentage based on the total weight of the immediate-release layer.

In some embodiments, the pharmaceutical formulation is a bilayer tablet, and the second layer is an extended-release layer, comprising the components with the following amounts (by weight):
(E-10) about 40-50% of the compound of formula (I), or a corresponding amount of the pharmaceutically acceptable salt thereof (or other glucokinase activator, or a prodrug thereof), about 8-10% of silicified microcrystalline cellulose (or other filler), about 24-33% of hypromellose K100M (or other release modifier), about 13-16% of sodium lauryl sulfate (or other surfactant), about 0.1-3% of colloidal silicone dioxide (or other glidant), and about 0.2-5% of magnesium stearate (or other lubricant); said percentage based on the total weight of the extended-release layer.

In some embodiments, the pharmaceutical formulation is a bilayer tablet, and the second layer is an extended-release layer, comprising the components with the following amounts (by weight):
(E-9) about 20-25% of the compound of formula (I), or a corresponding amount of the pharmaceutically acceptable salt thereof (or other glucokinase activator, or a prodrug thereof), about 28-35% of silicified microcrystalline cellulose (or other filler), about 24-33% of hypromellose K100M (or other release modifier), about 13-16% of sodium lauryl sulfate (or other surfactant), about 0.1-3% of colloidal silicone dioxide (or other glidant), and about 0.2-5% of magnesium stearate (or other lubricant); said percentage based on the total weight of the extended-release layer.

In some embodiments, the pharmaceutical formulation is a bilayer tablet, and the second layer is an extended-release layer, comprising the components with the following amounts (by weight):
(E-11) about 32-40% of the compound of formula (I), or a corresponding amount of the pharmaceutically acceptable salt thereof (or other glucokinase activator, or a prodrug thereof), about 7-10% of silicified microcrystalline cellulose (or other filler), about 26-33% of hypromellose K100M (or other release modifier), about 21-26% of sodium lauryl sulfate (or other surfactant), about 0.1-3% of colloidal silicone dioxide (or other glidant), and about 0.2-5% of magnesium stearate (or other lubricant), said percentage based on the total weight of the extended-release layer; said percentage based on the total weight of the extended-release layer.

In some embodiments, the pharmaceutical formulation is a bilayer tablet, and the second layer is an extended-release layer, comprising the components with the following amounts (by weight):
(E-12) about 32-40% of the compound of formula (I), or a corresponding amount of the pharmaceutically acceptable salt thereof (or other glucokinase activator, or a prodrug thereof), about 7-10% of silicified microcrystalline cellulose (or other filler), about 26-33% of hypromellose K4M (or other release modifier), about 21-26% of sodium lauryl sulfate (or other surfactant), about 0.1-3% of colloidal silicone dioxide (or other glidant), and about 0.2-5% of magnesium stearate (or other lubricant), said percentage based on the total weight of the extended-release layer; said percentage based on the total weight of the extended-release layer.

In some embodiments, the pharmaceutical formulation is a bilayer tablet, and the second layer is an extended-release layer, comprising the components with the following amounts (by weight):
(E-13) about 32-40% of the compound of formula (I), or a corresponding amount of the pharmaceutically acceptable salt thereof (or other glucokinase activator, or a prodrug thereof), about 7-10% of silicified microcrystalline cellulose (or other filler), about 26-33% of hypromellose K100LV (or other release modifier), about 21-26% of sodium lauryl sulfate (or other surfactant), about 0.1-3% of colloidal silicone dioxide (or other glidant), and about 0.2-5% of magnesium stearate (or other lubricant), said percentage based on the total weight of the extended-release layer; said percentage based on the total weight of the extended-release layer.

In some embodiments, the pharmaceutical formulation is a bilayer tablet, and the second layer is an extended-release layer, comprising the components with the following amounts (by weight):
(E-f) about 18-27% of the compound of formula (I), or a corresponding amount of the pharmaceutically acceptable salt thereof (or other glucokinase activator, or a prodrug thereof), about 25-38% of silicified microcrystalline cellulose (or other filler), about 25-35% of hypromellose K100M (or other release modifier), about 13-16% of sodium lauryl sulfate (or other surfactant), about 0.1-3% of colloidal silicone dioxide (or other glidant), and about 0.2-5% of magnesium stearate (or other lubricant), said percentage based on the total weight of the extended-release layer; said percentage based on the total weight of the extended-release layer.

In some embodiments, the immediate-release layer comprising:

(h)
about 18.22% of the compound of formula (I), or a corresponding amount of the pharmaceutically acceptable salt thereof;
about 73.11% of silicified microcrystalline cellulose;
about 3.09% of croscarmellose sodium;
about 3.64% of sodium lauryl sulfate;
about 0.55% of colloidal silicone dioxide; and
about 1.39% of magnesium stearate; said percentage based on the total weight of the immediate-release layer.

In some embodiments, the immediate-release layer comprising:

(i)
about 36.44% of the compound of formula (I), or a corresponding amount of the pharmaceutically acceptable salt thereof;
about 54.47% of silicified microcrystalline cellulose;
about 3.09% of croscarmellose sodium;
about 3.64% of sodium lauryl sulfate;
about 0.55% of colloidal silicone dioxide; and
about 1.81% of magnesium stearate;
   said percentage based on the total weight of the immediate-release layer.

In some embodiments, the immediate-release layer comprising:

(j)
about 36.18% of the compound of formula (I), or a corresponding amount of the pharmaceutically acceptable salt thereof;
about 51.38% of silicified microcrystalline cellulose;
about 3.09% of croscarmellose sodium;
about 7.06% of sodium lauryl sulfate;
about 0.53% of colloidal silicone dioxide; and
about 1.76% of magnesium stearate;
   said percentage based on the total weight of the immediate-release layer.

In some embodiments, the immediate-release layer comprising:

(i-f)
about 22.36% of the compound of formula (I), or a corresponding amount of the pharmaceutically acceptable salt thereof;
about 69.45% of silicified microcrystalline cellulose;
about 3.09% of croscarmellose sodium;
about 3.64% of sodium lauryl sulfate;
about 0.54% of colloidal silicone dioxide; and
about 0.91% of magnesium stearate;
   said percentage based on the total weight of the immediate-release layer.

In some embodiments, the extended-release layer comprising:
(E-10) about 44.73% of the compound of formula (I), or a corresponding amount of the pharmaceutically acceptable salt thereof; about 8.86% of silicified microcrystalline cellulose; about 29.82% of hypromellose K100M; about 14.55% of sodium lauryl sulfate; about 0.55% of colloidal silicone dioxide; and about 1.50% of magnesium stearate; said percentage based on the total weight of the extended-release layer.

In some embodiments, the extended-release layer comprising:
(E-9) about 22.36% of the compound of formula (I), or a corresponding amount of the pharmaceutically acceptable salt thereof; about 31.52% of silicified microcrystalline cellulose; about 29.82% of hypromellose K100M; about 14.55% of sodium lauryl sulfate; about 0.55% of colloidal silicone dioxide; and about 1.21% of magnesium stearate; said percentage based on the total weight of the extended-release layer.

In some embodiments, the extended-release layer comprising:
(E-11) about 36.18% of the compound of formula (I), or a corresponding amount of the pharmaceutically acceptable salt thereof; about 8.71% of silicified microcrystalline cellulose; about 29.59% of hypromellose K100M; about 23.53% of sodium lauryl sulfate; about 0.53% of colloidal silicone dioxide; and about 1.47% of magnesium stearate; said percentage based on the total weight of the extended-release layer.

In some embodiments, the extended-release layer comprising:
(E-12) about 36.18% of the compound of formula (I), or a corresponding amount of the pharmaceutically acceptable salt thereof; about 8.71% of silicified microcrystalline cellulose; about 29.59% of hypromellose K4M; about 23.53% of sodium lauryl sulfate; about 0.53% of colloidal silicone dioxide; and about 1.47% of magnesium stearate; said percentage based on the total weight of the extended-release layer.

In some embodiments, the extended-release layer comprising:
(E-13) about 36.18% of the compound of formula (I), or a corresponding amount of the pharmaceutically acceptable salt thereof; about 8.71% of silicified microcrystalline cellulose; about 29.59% of hypromellose K100LV; about 23.53% of sodium lauryl sulfate; about 0.53% of colloidal silicone dioxide; and about 1.47% of magnesium stearate; said percentage based on the total weight of the extended-release layer.

In some embodiments, the extended-release layer comprising:
(E-f) about 22.36% of the compound of formula (I), or a corresponding amount of the pharmaceutically acceptable salt thereof; about 31.82% of silicified microcrystalline cellulose; about 29.82% of hypromellose K100M; about 14.55% of sodium lauryl sulfate; about 0.54% of colloidal silicone dioxide; and about 0.91% of magnesium stearate, said percentage based on the total weight of the extended-release layer.

In some embodiments, the glucokinase activator is the compound of formula (I) or a corresponding amount of the pharmaceutically acceptable salt thereof.

In some embodiments, the pharmaceutical formulation (alternatively, bilayer tablet) comprises the glucokinase activator, or the prodrug in a dose (alternatively, a unit dose) ranging from about 1 mg to about 1500 mg, from about 1.23 mg to about 1230 mg, from about 1 mg to about 1000 mg, from about 5 mg to about 615 mg, from about 10 mg to about 492 mg, from about 15 mg to about 369 mg, from about 20 mg to about 246 mg, or from about 25 mg to about 246 mg, from about 50 mg to about 184 mg, or from about 75 mg to about 123 mg.

In some embodiments, the pharmaceutical formulation comprises the glucokinase activator, or the prodrug in a dose (alternatively, a unit dose) of about 10 mg, about 12.3 mg, about 18.4 mg, about 20 mg, about 24.6 mg, about 25 mg, about 30 mg, about 30.75 mg, about 35 mg, about 36.9 mg, about 40 mg, about 43.05 mg, about 45 mg, about 49.2 mg, about 50 mg, about 55 mg, about 55.35 mg, about 60 mg, about 61.5 mg, about 65 mg, about 67.65 mg, about 70 mg, about 73.8 mg, about 75 mg, about 79.95 mg, about 80 mg, about 85 mg, about 86.1 mg, about 90 mg, about 92 mg, about 95 mg, about 98.4 mg, about 100 mg, about 104.55 mg, about 110 mg, about 110.7 mg, about 116.85 mg, about 120 mg, about 123 mg, about 130 mg, about 135.3 mg, about 140 mg, about 147.6 mg, about 150 mg, about 159.9 mg, about 160 mg, about 170 mg, about 172.2 mg, about 180 mg, or about 184 mg, about 190 mg, about 196.8 mg, about 200 mg, about 209.1 mg, about 221.4 mg, about 233.7 mg, about 246 mg, about 250 mg, about 300 mg, about 307.5 mg, about 350 mg, about 369 mg, about 400 mg, about 430.5 mg, or about 492 mg.

In some embodiments, the pharmaceutical formulation (alternatively, bilayer tablet) comprises the compound of formula (I) or a corresponding amount of the pharmaceutically acceptable salt, the compound of formula (I) is in a dose (alternatively, a unit dose) ranging from about 1.23 mg to about 1230 mg, from about 6.15 mg to about 615 mg, from about 12.3 mg to about 492 mg, from about 18.45 mg to about 369 mg, from about 24.6 mg to about 246 mg, from about 30.75 mg to about 246 mg; from about 61.5 mg to about 184 mg; or from about 92 mg to about 123 mg.

In some embodiments, the compound of formula (I) is present in a dose (alternatively, a unit dose) of about 10 mg, about 12.3 mg, about 18.4 mg, about 20 mg, about 24.6 mg, about 25 mg, about 30 mg, about 30.75 mg, about 35 mg, about 36.9 mg, about 40 mg, about 43.05 mg, about 45 mg, about 49.2 mg, about 50 mg, about 55 mg, about 55.35 mg, about 60 mg, about 61.5 mg, about 65 mg, about 67.65 mg, about 70 mg, about 73.8 mg, about 75 mg, about 79.95 mg, about 80 mg, about 85 mg, about 86.1 mg, about 90 mg, about 92 mg, about 95 mg, about 98.4 mg, about 100 mg, about 104.55 mg, about 110 mg, about 110.7 mg, about 116.85 mg, about 120 mg, about 123 mg, about 130 mg, about 135.3 mg, about 140 mg, about 147.6 mg, about 150 mg, about 159.9 mg, about 160 mg, about 170 mg, about 172.2 mg, about 180 mg, about 184 mg, about 190 mg, about 196.8 mg, about 200 mg, about 209.1 mg, about 221.4 mg, about 233.7 mg, about 246 mg, about 307.5 mg, about 369 mg, about 430.5 mg, or about 492 mg.

In some embodiments, the compound of formula (I) is present in a dose (alternatively, a unit dose) of about 30.75 mg, about 61.5 mg, about 92 mg, about 123 mg, about 184 mg, about 246 mg, about 307.5 mg, about 369 mg, about 430.5 mg, or about 492 mg. In one embodiment, the dose is about 61.5 mg. In one embodiment, the dose is about 123 mg. In one embodiment, the dose is about 184.5 mg (or about 185 mg). In one embodiment, the dose is about 246 mg.

In some embodiments, the first part (alternatively, the first layer) is IR1 as described in Table 1. In some embodiments, the first part (alternatively, the first layer) is any one of IR2-IR7 as described in Table 2. In some embodiments, the first part (alternatively, the first layer) is any one of IR8-IR10 as described in Table 2-1. In some embodiments, the second part (alternatively, the second layer) is ER1 as described in Table 3. In some embodiments, the second part (alternatively, the second layer) is ER2 as described in Table 4. In some embodiments, the second part (alternatively, the second layer) is ER3 as described in Table 5. In some embodiments, the second part (alternatively, the second layer) is any one of ER4-ER8 as described in Table 6. In some embodiments, the second part (alternatively, the second layer) is any one of ER9-ER12 as described in Table 6-1.

In some embodiments, the weight ratio of the first part (alternatively, the first layer) to the second part (alternatively, the second layer) is about 1:10 to 10:1, about 1:9 to 9:1, about 1:4 to 4:1, about 3:7 to 7:3, about 2:3 to 3:2, about 3:4 to 4:3, about 4:5 to 5:4 or about 5:6 to 6:5; alternatively, the weight ratio of the first layer to the second layer is about 1:1, about 1:2, about 2:3, about 3:4, about 4:5, about 5:6, about 2:1, about 3:2, about 4:3, about 5:4 or about 6:5. In some embodiments, said weight ratio is about 2:3. In some embodiments, said weight ratio is about 45:55. In some embodiments, said weight ratio is about 1:1.

In some embodiments, the pharmaceutical formulation is a bilayer tablet, the first part is a first layer (e.g., an immediate-release layer), comprising:
about 16-20% of the compound of formula (I), or a corresponding amount of the pharmaceutically acceptable salt thereof;
about 66-81% of filler(s) (e.g., silicified microcrystalline cellulose);
about 2.5-3.5% of disintegrant(s) (e.g., croscarmellose sodium);
about 34% of surfactant(s) (e.g., sodium lauryl sulfate);
about 0.1-3% of glidant(s) (e.g., colloidal silicone dioxide); and
about 0.2-5% of lubricant(s) (e.g., magnesium stearate);
   said percentage based on the total weight of the first layer;
   the second part is a second layer (e.g., an extended-release layer), comprising:
about 20-25% of the compound of formula (I), or a pharmaceutically acceptable salt thereof;
about 28-35% of filler(s) (e.g., silicified microcrystalline cellulose);
about 24-33% of release modifier(s) (e.g., hypromellose K100M);
about 13-16% of surfactant(s) (e.g., sodium lauryl sulfate);
about 0.1-3% of glidant(s) (e.g., colloidal silicone dioxide); and
about 0.2-5% of lubricant(s) (e.g., magnesium stearate);
   said percentage based on the total weight of the second layer;
   wherein the weight ratio of the compound of formula (I) in the first part to the compound of formula (I) in the second part is from about 1:4 to about 3:2;
   optionally the total weight of the compound of formula (I) in the pharmaceutical formulation is no more than about 92.25 mg (e.g., is about 61.5 mg).

In some embodiments, the pharmaceutical formulation is a bilayer tablet, the first part is a first layer (e.g., an immediate-release layer), comprising:
about 18.22% of the compound of formula (I), or a corresponding amount of the pharmaceutically acceptable salt thereof;
about 73.11% of filler(s) (e.g., silicified microcrystalline cellulose);
about 3.09% of disintegrant(s) (e.g., croscarmellose sodium);
about 3.64% of surfactant(s) (e.g., sodium lauryl sulfate);
about 0.55% of glidant(s) (e.g., colloidal silicone dioxide); and
about 1.39% of lubricant(s) (e.g., magnesium stearate);
   said percentage based on the total weight of the first layer;
   the second part is a second layer (e.g., an extended-release layer), comprising:
about 22.36% of the compound of formula (I), or a corresponding amount of the pharmaceutically acceptable salt thereof;
about 31.52% of filler(s) (e.g., silicified microcrystalline cellulose);
about 29.82% of release modifier(s) (e.g., hypromellose K100M);
about 14.55% of surfactant(s) (e.g., sodium lauryl sulfate);
about 0.55% of glidant(s) (e.g., colloidal silicone dioxide); and
about 1.21% of lubricant(s) (e.g., magnesium stearate);
   said percentage based on the total weight of the second layer;
   wherein the weight ratio of the compound of formula (I) in the first part to the compound of formula (I) in the second part is from about 1:4 to about 3:2;
   optionally the total weight of the compound of formula (I) in the pharmaceutical formulation is no more than about 92.25 mg (e.g., is about 61.5 mg).

In some embodiments, the pharmaceutical formulation is a bilayer tablet, the first part is a first layer (e.g., an immediate-release layer), comprising:
about 32-40% of the compound of formula (I), or a corresponding amount of the pharmaceutically acceptable salt thereof (or other glucokinase activator, or a prodrug thereof);
about 49-60% of filler(s) (e.g., silicified microcrystalline cellulose);
about 2.5-3.5% of disintegrant(s) (e.g., croscarmellose sodium);
about 3-4% of surfactant(s) (e.g., sodium lauryl sulfate);
about 0.1-3% of glidant(s) (e.g., colloidal silicone dioxide); and
about 0.2-5% of lubricant(s) (e.g., magnesium stearate);
   said percentage based on the total weight of the first layer;
   the second part is a second layer (e.g., an extended-release layer), comprising:
about 40-50% of the compound of formula (I), or a pharmaceutically acceptable salt thereof;
about 8-10% of filler(s) (e.g., silicified microcrystalline cellulose);
about 24-33% of release modifier(s) (e.g., hypromellose K100M);
about 13-16% of surfactant(s) (e.g., sodium lauryl sulfate);
about 0.1-3% of glidant(s) (e.g., colloidal silicone dioxide); and
about 0.2-5% of lubricant(s) (e.g., magnesium stearate);
   said percentage based on the total weight of the second layer;
   wherein the weight ratio of the compound of formula (I) in the first part to the compound of formula (I) in the second part is from about 1:4 to about 3:2;
   optionally the total weight of the compound of formula (I) in the pharmaceutical formulation is from about 92.25 mg to about 153.75 mg (e.g., is about 123 mg).

In some embodiments, the pharmaceutical formulation is a bilayer tablet, the first part is a first layer (e.g., an immediate-release layer), comprising:
about 36.44% of the compound of formula (I), or a corresponding amount of the pharmaceutically acceptable salt thereof;
about 54.47% of filler(s) (e.g., silicified microcrystalline cellulose);
about 3.09% of disintegrant(s) (e.g., croscarmellose sodium);
about 3.64% of surfactant(s) (e.g., sodium lauryl sulfate);
about 0.55% of glidant(s) (e.g., colloidal silicone dioxide); and
about 1.81% of lubricant(s) (e.g., magnesium stearate);
   said percentage based on the total weight of the first layer;
   the second part is a second layer (e.g., an extended-release layer), comprising:
about 44.73% of the compound of formula (I), or a corresponding amount of the pharmaceutically acceptable salt thereof;
about 8.86% of filler(s) (e.g., silicified microcrystalline cellulose);
about 29.82% of release modifier(s) (e.g., hypromellose K100M);
about 14.55% of surfactant(s) (e.g., sodium lauryl sulfate);
about 0.55% of glidant(s) (e.g., colloidal silicone dioxide); and
about 1.50% of lubricant(s) (e.g., magnesium stearate);
   said percentage based on the total weight of the second layer;
   wherein the weight ratio of the compound of formula (I) in the first part to the compound of formula (I) in the second part is from about 1:4 to about 3:2;
   optionally the total weight of the compound of formula (I) in the pharmaceutical formulation is from about 92.25 mg to about 153.75 mg (e.g., is about 123 mg).

In some embodiments, the pharmaceutical formulation is a bilayer tablet, the first part is a first layer (e.g., an immediate-release layer), comprising:
about 32-40% of the compound of formula (I), or a corresponding amount of the pharmaceutically acceptable salt thereof (or other glucokinase activator, or a prodrug thereof);
about 46-57% of filler(s) (e.g., silicified microcrystalline cellulose);
about 2.5-3.5% of disintegrant(s) (e.g., croscarmellose sodium);
about 6-8% of surfactant(s) (e.g., sodium lauryl sulfate);
about 0.1-3% of colloidal silicone dioxide (or other glidant); and
about 0.2-5% of lubricant(s) (e.g., magnesium stearate);
   said percentage based on the total weight of the first layer;
   the second part is a second layer (e.g., an extended-release layer), comprising:
about 32-40% of the compound of formula (I), or a pharmaceutically acceptable salt thereof;
about 7-10% of filler(s) (e.g., silicified microcrystalline cellulose);
about 26-33% of release modifier(s) (e.g., hypromellose K100M, hypromellose K4M, or hypromellose K100LV);
about 21-26% of surfactant(s) (e.g., sodium lauryl sulfate);
about 0.1-3% of glidant(s) (e.g., colloidal silicone dioxide); and
about 0.2-5% of lubricant(s) (e.g., magnesium stearate);
   said percentage based on the total weight of the second layer;
   wherein the weight ratio of the compound of formula (I) in the first part to the compound of formula (I) in the second part is from about 1:4 to about 3:2;
   optionally the total weight of the compound of formula (I) in the pharmaceutical formulation is from about 92.25 mg to about 153.75 mg (e.g., is about 123 mg), from about 153.75 mg to about 215.25 mg (e.g., is about 184.5 mg), or no less than about 215.25 mg, or is from about 215.25 mg to about 276.75 mg (e.g., is about 246 mg).

In some embodiments, the pharmaceutical formulation is a bilayer tablet, the first part is a first layer (e.g., an immediate-release layer), comprising:
about 36.18% of the compound of formula (I), or a corresponding amount of the pharmaceutically acceptable salt thereof;
about 51.38% of filler(s) (e.g., silicified microcrystalline cellulose);
about 3.09% of disintegrant(s) (e.g., croscarmellose sodium);
about 7.06% of surfactant(s) (e.g., sodium lauryl sulfate);
about 0.53% of glidant(s) (e.g., colloidal silicone dioxide); and
about 1.76% of lubricant(s) (e.g., magnesium stearate);
   said percentage based on the total weight of the first layer;
   the second part is a second layer (e.g., an extended-release layer), comprising:
about 36.18% of the compound of formula (I), or a corresponding amount of the pharmaceutically acceptable salt thereof;
about 8.71% of filler(s) (e.g., silicified microcrystalline cellulose);
about 29.59% of release modifier(s) (e.g., hypromellose K100M, hypromellose K4M, or hypromellose K100LV);
about 23.53% of surfactant(s) (e.g., sodium lauryl sulfate);
about 0.53% of glidant(s) (e.g., colloidal silicone dioxide); and
about 1.47% of lubricant(s) (e.g., magnesium stearate);
   said percentage based on the total weight of the second layer;
   wherein the weight ratio of the compound of formula (I) in the first part to the compound of formula (I) in the second part is from about 1:4 to about 3:2;
   optionally the total weight of the compound of formula (I) in the pharmaceutical formulation is from about 92.25 mg to about 153.75 mg (e.g., is about 123 mg), from about 153.75 mg to about 215.25 mg (e.g., is about 184.5 mg), or no less than about 215.25 mg, or is from about 215.25 mg to about 276.75 mg (e.g., is about 246 mg).

In some embodiments, the weight ratio of the compound of formula (I) in the first part to the compound of formula (I) in the second part is from about 2:3 to about 1:1, or is about 2:3, or about 1:1. In some embodiments, the weight ratio is about 2:3. In some embodiments, the weight ratio is about 1:1.

In some embodiments, the weight ratio of the first part to the second part is from about 1:4 to about 3:2, or from about 2:3 to about 1:1, or is about 2:3, about 45:55, or about 1:1. In some embodiments, the weight ratio is from about 2:3 to about 1:1. In some embodiments, the weight ratio is about 2:3. In some embodiments, the weight ratio is about 44:55. In some embodiments, the weight ratio is about 1:1.

In some embodiments, the total weight of the compound of formula (I) in the pharmaceutical formulation is no more than about 92.25 mg (e.g., is about 61.5 mg), and the release modifier is a hypromellose having viscosity no more than about 140,000 mPa·s, from about 75,000 to about 140,000 mPa·s, or about 100,000 mPa·s (e.g., hypromellose K100M).

In some embodiments, the total weight of the compound of formula (I) in the pharmaceutical formulation is from about 92.25 mg to about 153.75 mg (e.g., is about 123 mg), and the release modifier is a hypromellose having viscosity no more than about 140,000 mPa·s, from about 75,000 to about 140,000 mPa·s, or about 100,000 mPa·s (e.g., hypromellose K100M).

In some embodiments, the total weight of the compound of formula (I) in the pharmaceutical formulation is from about 92.25 mg to about 153.75 mg (e.g., is about 123 mg), and the release modifier is a hypromellose having viscosity from about 100 to about 20,000 mPa·s, from about 1000 to about 10,000 mPa·s, or about 4000 mPa·s (e.g., hypromellose K4M).

In some embodiments, the total weight of the compound of formula (I) in the pharmaceutical formulation is from about 153.75 mg to about 215.25 mg (e.g., is about 184.5 mg), and the release modifier is a hypromellose having viscosity no more than about 140,000 mPa·s, from about 75,000 to about 140,000 mPa·s, or about 100,000 mPa·s (e.g., hypromellose K100M).

In some embodiments, the total weight of the compound of formula (I) in the pharmaceutical formulation is from about 153.75 mg to about 215.25 mg (e.g., is about 184.5 mg), and the release modifier is a hypromellose having viscosity from about 100 to about 20,000 mPa·s, from about 1000 to about 10,000 mPa·s, or about 4000 mPa·s (e.g., hypromellose K4M).

In some embodiments, the total weight of the compound of formula (I) in the pharmaceutical formulation is no less than about 215.25 mg, or is from about 215.25 mg to about 276.75 mg (e.g., is about 246 mg), and the release modifier is a hypromellose having viscosity from about 100 to about 20,000 mPa·s, from about 1000 to about 10,000 mPa·s, or about 4000 mPa·s (e.g., hypromellose K4M).

In some embodiments, the total weight of the compound of formula (I) in the pharmaceutical formulation is no less than about 215.25 mg, or is from about 215.25 mg to about 276.75 mg (e.g., is about 246 mg), and the release modifier is a hypromellose having viscosity from about 1 to about 1000 mPa·s, from about 1 to about 500 mPa·s, or about 100 mPa·s (e.g., hypromellose K100LV).

In some embodiments, the first layer comprises:
about 10-100 mg of the compound of formula (I), or a corresponding amount of the pharmaceutically acceptable salt thereof;
about 30-150 mg of silicified microcrystalline cellulose;
about 0-10 mg of croscarmellose sodium;
about 0-10 mg of sodium lauryl sulfate;
about 0-5 mg of colloidal silicone dioxide; and
about 0.1-10 mg of magnesium stearate.

In some embodiments, the first layer comprises:

(a)
about 40-61.5 mg of the compound of formula (I), or a corresponding amount of the pharmaceutically acceptable salt thereof;
about 40-65 mg of silicified microcrystalline cellulose;
about 2-5 mg of croscarmellose sodium;
about 2-15 mg of sodium lauryl sulfate;
about 0.1-3 mg of colloidal silicone dioxide; and
about 0.1-5 mg of magnesium stearate.

In some embodiments, the first layer comprises:

(b)
about 30-45 mg of the compound of formula (I), or a corresponding amount of the pharmaceutically acceptable salt thereof;
about 50-85 mg of silicified microcrystalline cellulose;
about 2-5 mg of croscarmellose sodium; and
about 0.1-3 mg of magnesium stearate.

In some embodiments, the first layer comprises:

(c)
about 20-30 mg of the compound of formula (I), or a corresponding amount of the pharmaceutically acceptable salt thereof;
about 60-95 mg of silicified microcrystalline cellulose;
about 2-5 mg of croscarmellose sodium;
about 2-15 mg of sodium lauryl sulfate;
about 0.1-3 mg of colloidal silicone dioxide; and
about 0.1-5 mg of magnesium stearate.

In some embodiments, the first layer comprises:

(d)
about 10-15 mg of the compound of formula (I), or a corresponding amount of the pharmaceutically acceptable salt thereof;
about 70-110 mg of silicified microcrystalline cellulose;
about 2-5 mg of croscarmellose sodium;
about 2-15 mg of sodium lauryl sulfate;
about 0.1-3 mg of colloidal silicone dioxide; and
about 0.1-5 mg of magnesium stearate.

In some embodiments, the second layer comprises:

### (A)

about 60-90 mg of the compound of formula (I), or a corresponding amount of the pharmaceutically acceptable salt thereof;
about 10-30 mg of silicified microcrystalline cellulose;
about 35-60 mg of hypromellose K100M;
about 15-30 mg of sodium lauryl sulfate;
about 0.1-3 mg of colloidal silicone dioxide; and
about 0.1-5 mg of magnesium stearate.

In some embodiments, the second layer comprises:

### (B)

about 60-90 mg of the compound of formula (I), or a corresponding amount of the pharmaceutically acceptable salt thereof;
about 20-40 mg of silicified microcrystalline cellulose;
about 35-60 mg of hypromellose K100M;
about 3-15 mg of sodium lauryl sulfate;
about 0.1-3 mg of colloidal silicone dioxide; and
about 0.1-5 mg of magnesium stearate.

In some embodiments, the second layer comprises:

### (C)

about 60-90 mg of the compound of formula (I), or a corresponding amount of the pharmaceutically acceptable salt thereof;
about 10-30 mg of silicified microcrystalline cellulose;
about 35-60 mg of hypromellose E5;
about 15-30 mg of sodium lauryl sulfate;
about 0.1-3 mg of colloidal silicone dioxide; and
about 0.1-5 mg of magnesium stearate.

In some embodiments, the second layer comprises:

### (D)

about 60-90 mg of the compound of formula (I), or a corresponding amount of the pharmaceutically acceptable salt thereof;
about 10-30 mg of silicified microcrystalline cellulose;
about 15-30 mg of hypromellose E5;
about 15-30 mg of hypromellose K100LV;
about 15-30 mg of sodium lauryl sulfate;
about 0.1-3 mg of colloidal silicone dioxide; and
about 0.1-5 mg of magnesium stearate.

In some embodiments, the second layer comprises:

### (E)

about 60-90 mg of the compound of formula (I), or a corresponding amount of the pharmaceutically acceptable salt thereof;
about 10-30 mg of silicified microcrystalline cellulose;
about 35-60 mg of hypromellose K100LV
about 15-30 mg of sodium lauryl sulfate;
about 0.1-3 mg of colloidal silicone dioxide; and
about 0.1-5 mg of magnesium stearate.

In some embodiments, the second layer comprises:

### (F)

about 60-90 mg of the compound of formula (I) or a corresponding amount of the pharmaceutically acceptable salt thereof;
about 30-50 mg of silicified microcrystalline cellulose;
about 35-60 mg of hypromellose K100M
about 0.1-3 mg of colloidal silicone dioxide; and
about 0.1-5 mg of magnesium stearate.

In some embodiments, the first layer comprises:

(a)
about 49.2 mg of the compound of formula (I), or a corresponding amount of the pharmaceutically acceptable salt thereof;
about 51.8 mg of silicified microcrystalline cellulose;
about 3.4 mg of croscarmellose sodium;
about 4.0 mg of sodium lauryl sulfate;
about 0.6 mg of colloidal silicone dioxide; and
about 1.0 mg of magnesium stearate.

In some embodiments, the first layer comprises:

(b)
about 36.8 mg of the compound of formula (I), or a corresponding amount of the pharmaceutically acceptable salt thereof;
about 68.68 mg of silicified microcrystalline cellulose;
about 3.4 mg of croscarmellose sodium; and
about 1.12 mg of magnesium stearate.

In some embodiments, the first layer comprises:

(c)
about 24.6 mg of the compound of formula (I), or a corresponding amount of the pharmaceutically acceptable salt thereof;
about 76.4 mg of silicified microcrystalline cellulose;
about 3.4 mg of croscarmellose sodium;
about 4.0 mg of sodium lauryl sulfate;
about 0.6 mg of colloidal silicone dioxide; and
about 1.0 mg of magnesium stearate.

In some embodiments, the first layer comprises:

(d)
about 12.3 mg of the compound of formula (I), or a corresponding amount of the pharmaceutically acceptable salt thereof;
about 88.7 mg of silicified microcrystalline cellulose;
about 3.4 mg of croscarmellose sodium;
about 4.0 mg of sodium lauryl sulfate;
about 0.6 mg of colloidal silicone dioxide; and
about 1.0 mg of magnesium stearate.

In some embodiments, the second layer comprises:

### (A)

about 73.8 mg of the compound of formula (I), or a corresponding amount of the pharmaceutically acceptable salt thereof;
about 15.6 mg of silicified microcrystalline cellulose;
about 49.2 mg of hypromellose K100M;
about 24 mg of sodium lauryl sulfate;
about 0.9 mg of colloidal silicone dioxide; and
about 1.5 mg of magnesium stearate.

In some embodiments, the second layer comprises:

### (B)

about 73.8 mg of the compound of formula (I), or a corresponding amount of the pharmaceutically acceptable salt thereof;
about 33.6 mg of silicified microcrystalline cellulose;
about 49.2 mg of hypromellose K100M;
about 6.0 mg of sodium lauryl sulfate;
about 0.9 mg of colloidal silicone dioxide; and
about 1.5 mg of magnesium stearate.

In some embodiments, the second layer comprises:

### (C)

about 73.8 mg of the compound of formula (I), or a corresponding amount of the pharmaceutically acceptable salt thereof;
about 15.6 mg of silicified microcrystalline cellulose;
about 49.2 mg of hypromellose E5;
about 24 mg of sodium lauryl sulfate;
about 0.9 mg of colloidal silicone dioxide; and
about 1.5 mg of magnesium stearate.

In some embodiments, the second layer comprises:

### (D)

about 73.8 mg of the compound of formula (I), or a corresponding amount of the pharmaceutically acceptable salt thereof;
about 15.6 mg of silicified microcrystalline cellulose;
about 24.6 mg of hypromellose E5;
about 24.6 mg of hypromellose K100LV;
about 24 mg of sodium lauryl sulfate;
about 0.9 mg of colloidal silicone dioxide; and
about 1.5 mg of magnesium stearate.

In some embodiments, the second layer comprises:

### (E)

about 73.8 mg of the compound of formula (I), or a corresponding amount of the pharmaceutically acceptable salt thereof;
about 15.6 mg of silicified microcrystalline cellulose;
about 49.2 mg of hypromellose K100LV;
about 24 mg of sodium lauryl sulfate;
about 0.9 mg of colloidal silicone dioxide; and
about 1.5 mg of magnesium stearate.

In some embodiments, the second layer comprises:

### (F)

about 73.8 mg of the compound of formula (I) or a corresponding amount of the pharmaceutically acceptable salt thereof;
about 39.6 mg of silicified microcrystalline cellulose;
about 49.2 mg of hypromellose K100M
about 0.9 mg of colloidal silicone dioxide; and
about 1.5 mg of magnesium stearate.

In some embodiments, the first layer comprises:
about 15-265 mg of the compound of formula (I), or a corresponding amount of the pharmaceutically acceptable salt thereof;
about 30-250 mg of silicified microcrystalline cellulose;
about 0-15 mg of croscarmellose sodium;
about 0-35 mg of sodium lauryl sulfate;
about 0-5 mg of colloidal silicone dioxide; and
about 0.1-10 mg of magnesium stearate.

In some embodiments, the first layer comprises:
about 15-150 mg of the compound of formula (I), or a corresponding amount of the pharmaceutically acceptable salt thereof;
about 50-210 mg of silicified microcrystalline cellulose;
about 1-15 mg of croscarmellose sodium;
about 1-30 mg of sodium lauryl sulfate;
about 0-5 mg of colloidal silicone dioxide; and
about 0-10 mg of magnesium stearate.

In some embodiments, the first layer comprises:

(e)
about 19-30 mg the compound of formula (I), or a corresponding amount of the pharmaceutically acceptable salt thereof;
about 78-120 mg silicified microcrystalline cellulose;
about 3-6 mg croscarmellose sodium;
about 3-6 mg sodium lauryl sulfate;
about 0.1-3 mg colloidal silicone dioxide; and
about 1.5-6 mg of magnesium stearate.

In some embodiments, the first layer comprises:

(f)
about 39-60 mg the compound of formula (I), or a corresponding amount of the pharmaceutically acceptable salt thereof;
about 56-89 mg silicified microcrystalline cellulose;
about 2-5 mg croscarmellose sodium;
about 4-12 mg sodium lauryl sulfate;
about 0.1-3 mg colloidal silicone dioxide; and
about 1.5-6 mg of magnesium stearate.

In some embodiments, the first layer comprises:

(g)
about 58-120 mg the compound of formula (I), or a corresponding amount of the pharmaceutically acceptable salt thereof;
about 83-168 mg silicified microcrystalline cellulose;
about 5-10 mg croscarmellose sodium;
about 11-23 mg sodium lauryl sulfate;
about 0.1-3 mg colloidal silicone dioxide; and
about 1.5-6 mg of magnesium stearate.

In some embodiments, the first layer comprises:

(h)
about 98-148 mg of the compound of formula (I), or a corresponding amount of the pharmaceutically acceptable salt thereof;
about 139-210 mg of silicified microcrystalline cellulose;
about 8-13 mg of croscarmellose sodium;
about 19-30 mg of sodium lauryl sulfate;
about 0.1-3 mg of colloidal silicone dioxide; and
about 4-8 mg of magnesium stearate;

In some embodiments, the second layer comprises:

(G) about 29-45 mg the compound of formula (I), or a corresponding amount of the pharmaceutically acceptable salt thereof;
about 41-63 mg silicified microcrystalline cellulose;
about 39-60 mg hypromellose K100M;
about 19-30 mg of sodium lauryl sulfate;
about 0.1-3 mg of colloidal silicone dioxide; and
about 1-5 mg of magnesium stearate.

In some embodiments, the second layer comprises:

### (H)

about 60-90 mg of the compound of formula (I), or a corresponding amount of the pharmaceutically acceptable salt thereof;
about 11-18 mg silicified microcrystalline cellulose;
about 39-60 mg hypromellose K100M;
about 19-30 mg sodium lauryl sulfate;
about 0.1-3 mg colloidal silicone dioxide; and
about 1-5 mg magnesium stearate.

In some embodiments, the second layer comprises:

### (I)

about 60-90 mg of the compound of formula (I), or a corresponding amount of the pharmaceutically acceptable salt thereof;
about 14-22 mg silicified microcrystalline cellulose;
about 48-73 mg hypromellose K100M;
about 38-58 mg sodium lauryl sulfate;
about 0.1-3 mg colloidal silicone dioxide; and
about 1-5 mg magnesium stearate.

In some embodiments, the second layer comprises:

### (J)

about 60-90 mg of the compound of formula (I), or a corresponding amount of the pharmaceutically acceptable salt thereof;
about 14-22 mg silicified microcrystalline cellulose;
about 48-73 mg hypromellose K4M;
about 38-58 mg sodium lauryl sulfate;
about 0.1-3 mg colloidal silicone dioxide; and
about 1-5 mg magnesium stearate.

In some embodiments, the second layer comprises:

### (K1)

about 88-133 mg of the compound of formula (I), or a corresponding amount of the pharmaceutically acceptable salt thereof;
about 21-32 mg silicified microcrystalline cellulose;
about 72-110 mg hypromellose K100M;
about 58-86 mg sodium lauryl sulfate;
about 0.1-3 mg colloidal silicone dioxide; and
about 3-6 mg magnesium stearate.

In some embodiments, the second layer comprises:

### (L)

about 88-133 mg of the compound of formula (I), or a corresponding amount of the pharmaceutically acceptable salt thereof;
about 21-32 mg silicified microcrystalline cellulose;
about 72-110 mg hypromellose K4M;
about 58-86 mg sodium lauryl sulfate;
about 0.1-3 mg colloidal silicone dioxide; and
about 3-6 mg magnesium stearate.

In some embodiments, the second layer comprises:

### (M)

about 118-178 mg of the compound of formula (I), or a corresponding amount of the pharmaceutically acceptable salt thereof;
about 28-43 mg silicified microcrystalline cellulose;
about 96-145 mg hypromellose K4M;
about 76-116 mg sodium lauryl sulfate;
about 0.1-3 mg colloidal silicone dioxide; and
about 4-8 mg magnesium stearate.

In some embodiments, the second layer comprises:

### (N)

about 118-178 mg of the compound of formula (I), or a corresponding amount of the pharmaceutically acceptable salt thereof;
about 28-43 mg silicified microcrystalline cellulose;
about 96-145 mg hypromellose K100LV;
about 76-116 mg sodium lauryl sulfate;
about 0.1-3 mg colloidal silicone dioxide; and
about 4-8 mg magnesium stearate.

In some embodiments, the second layer comprises:

### (O)

about 98-148 mg of the compound of formula (I), or a corresponding amount of the pharmaceutically acceptable salt thereof;
about 23-36 mg silicified microcrystalline cellulose;
about 80-121 mg hypromellose K100LV;
about 64-96 mg sodium lauryl sulfate;
about 0.1-3 mg colloidal silicone dioxide; and
about 3-7 mg magnesium stearate.

In some embodiments, the first layer comprises:

(e)
about 24.6 mg the compound of formula (I), or a corresponding amount of the pharmaceutically acceptable salt thereof;
about 98.70 mg silicified microcrystalline cellulose;
about 4.17 mg croscarmellose sodium;
about 4.91 mg sodium lauryl sulfate;
about 0.74 mg colloidal silicone dioxide; and
about 1.88 mg of magnesium stearate.

In some embodiments, the first layer comprises:

(f)
about 49.20 mg the compound of formula (I), or a corresponding amount of the pharmaceutically acceptable salt thereof;
about 73.54 mg silicified microcrystalline cellulose;
about 4.17 mg croscarmellose sodium;
about 4.91 mg sodium lauryl sulfate;
about 0.74 mg colloidal silicone dioxide; and
about 2.44 mg of magnesium stearate.

In some embodiments, the first layer comprises:

(g)
about 49.20 mg the compound of formula (I), or a corresponding amount of the pharmaceutically acceptable salt thereof;
about 69.88 mg silicified microcrystalline cellulose;
about 4.20 mg croscarmellose sodium;
about 9.60 mg sodium lauryl sulfate;
about 0.72 mg colloidal silicone dioxide; and
about 2.40 mg of magnesium stearate.

In some embodiments, the first layer comprises:

(h)
about 73.80 mg the compound of formula (I), or a corresponding amount of the pharmaceutically acceptable salt thereof;
about 104.82 mg silicified microcrystalline cellulose;
about 6.30 mg croscarmellose sodium;
about 14.40 mg sodium lauryl sulfate;
about 1.08 mg colloidal silicone dioxide; and
about 3.6 mg of magnesium stearate.

In some embodiments, the first layer comprises:

(i)
about 98.40 mg the compound of formula (I), or a corresponding amount of the pharmaceutically acceptable salt thereof;
about 139.76 mg silicified microcrystalline cellulose;
about 8.40 mg croscarmellose sodium;
about 19.20 mg sodium lauryl sulfate;
about 1.44 mg colloidal silicone dioxide; and
about 4.8 mg of magnesium stearate.

In some embodiments, the first layer comprises:

(j)
about 123.00 mg the compound of formula (I), or a corresponding amount of the pharmaceutically acceptable salt thereof;
about 174.70 mg silicified microcrystalline cellulose;
about 10.50 mg croscarmellose sodium;
about 24.00 mg sodium lauryl sulfate;
about 1.80 mg colloidal silicone dioxide; and
about 6.00 mg of magnesium stearate;
In some embodiments, the second layer comprises:

### (G)

about 36.90 mg the compound of formula (I), or a corresponding amount of the pharmaceutically acceptable salt thereof;
about 52.00 mg silicified microcrystalline cellulose;
about 49.20 mg hypromellose K100M;
about 24.00 mg of sodium lauryl sulfate;
about 0.90 mg of colloidal silicone dioxide; and
about 2.00 mg of magnesium stearate.

In some embodiments, the second layer comprises:

### (H)

about 73.80 mg of the compound of formula (I), or a corresponding amount of the pharmaceutically acceptable salt thereof;
about 14.62 mg silicified microcrystalline cellulose;
about 49.20 mg hypromellose K100M;
about 24.00 mg sodium lauryl sulfate;
about 0.90 mg colloidal silicone dioxide; and
about 2.48 mg magnesium stearate.

In some embodiments, the second layer comprises:

### (I)

about 73.80 mg of the compound of formula (I), or a corresponding amount of the pharmaceutically acceptable salt thereof;
about 17.76 mg silicified microcrystalline cellulose;
about 60.36 mg hypromellose K100M;
about 48.00 mg sodium lauryl sulfate;
about 1.08 mg colloidal silicone dioxide; and
about 3.00 mg magnesium stearate.

In some embodiments, the second layer comprises:

### (J)

about 73.80 mg of the compound of formula (I), or a corresponding amount of the pharmaceutically acceptable salt thereof;
about 17.76 mg silicified microcrystalline cellulose;
about 60.36 mg hypromellose K4M;
about 48.00 mg sodium lauryl sulfate;
about 1.08 mg colloidal silicone dioxide; and
about 3.00 mg magnesium stearate.

In some embodiments, the second layer comprises:

### (K1)

about 110.70 mg of the compound of formula (I), or a corresponding amount of the pharmaceutically acceptable salt thereof;
about 26.64 mg silicified microcrystalline cellulose;
about 90.54 mg hypromellose K100M;
about 72.00 mg sodium lauryl sulfate;
about 1.62 mg colloidal silicone dioxide; and
about 4.50 mg magnesium stearate.

In some embodiments, the second layer comprises:

### (L)

about 110.70 mg of the compound of formula (I), or a corresponding amount of the pharmaceutically acceptable salt thereof;
about 26.64 mg silicified microcrystalline cellulose;
about 90.54 mg hypromellose K4M;
about 72.00 mg sodium lauryl sulfate;
about 1.62 mg colloidal silicone dioxide; and
about 4.50 mg magnesium stearate.

In some embodiments, the second layer comprises:

### (M)

about 147.60 mg of the compound of formula (I), or a corresponding amount of the pharmaceutically acceptable salt thereof;
about 35.52 mg silicified microcrystalline cellulose;
about 120.72 mg hypromellose K4M;
about 96.00 mg sodium lauryl sulfate;
about 2.16 mg colloidal silicone dioxide; and
about 6.00 mg magnesium stearate.

In some embodiments, the second layer comprises:

### (N)

about 147.60 mg of the compound of formula (I), or a corresponding amount of the pharmaceutically acceptable salt thereof;
about 35.52 mg silicified microcrystalline cellulose;
about 120.72 mg hypromellose K100LV;
about 96.00 mg sodium lauryl sulfate;
about 2.16 mg colloidal silicone dioxide; and
about 6.00 mg magnesium stearate.

In some embodiments, the second layer comprises:

### (O)

about 123.00 mg of the compound of formula (I), or a corresponding amount of the pharmaceutically acceptable salt thereof;
about 29.60 mg silicified microcrystalline cellulose;
about 100.60 mg hypromellose K100LV;
about 80.00 mg sodium lauryl sulfate;
about 1.80 mg colloidal silicone dioxide; and
about 5.00 mg magnesium stearate.

In some specific embodiments, the pharmaceutical formulation is a bilayer tablet, wherein the formulation is a bilayer tablet, the first part is an immediate-release layer, comprising:
about 40-61.5 mg of the compound of formula (I), or a corresponding amount of the pharmaceutically acceptable salt thereof;
about 40-65 mg of silicified microcrystalline cellulose;
about 2-5 mg of croscarmellose sodium;
about 2-15 mg of sodium lauryl sulfate;
about 0.1-3 mg of colloidal silicone dioxide; and
about 0.1-5 mg of magnesium stearate;
   the second part is an extended-release layer, comprising:
about 60-90 mg of the compound of formula (I), or a corresponding amount of the pharmaceutically acceptable salt thereof;
about 10-30 mg of silicified microcrystalline cellulose;
about 35-60 mg of hypromellose K100M;
about 15-30 mg of sodium lauryl sulfate;
about 0.1-3 mg of colloidal silicone dioxide; and
about 0.1-5 mg of magnesium stearate.

In some embodiments, the pharmaceutical formulation is a bilayer tablet, wherein the formulation is a bilayer tablet, the first part is an immediate-release layer, comprising:
about 40-61.5 mg of the compound of formula (I), or a corresponding amount of the pharmaceutically acceptable salt thereof;
about 40-65 mg of silicified microcrystalline cellulose;
about 2-5 mg of croscarmellose sodium;
about 2-15 mg of sodium lauryl sulfate;
about 0.1-3 mg of colloidal silicone dioxide; and
about 0.1-5 mg of magnesium stearate;
   the second part is an extended-release layer, comprising:
about 60-90 mg of the compound of formula (I) or a corresponding amount of the pharmaceutically acceptable salt thereof;
about 20-40 mg of silicified microcrystalline cellulose;
about 35-60 mg of hypromellose K100M;
about 3-15 mg of sodium lauryl sulfate;
about 0.1-3 mg of colloidal silicone dioxide; and
about 0.1-5 mg of magnesium stearate.

In some embodiments, the pharmaceutical formulation is a bilayer tablet, the first part is an immediate-release layer, comprising:
about 20-30 mg of the compound of formula (I), or the pharmaceutically acceptable salt thereof;
about 61-92 mg of silicified microcrystalline cellulose;
about 2-5 mg of croscarmellose sodium;
about 2-15 mg of sodium lauryl sulfate;
about 0.1-3 mg of colloidal silicone dioxide; and
about 0.1-5 mg of magnesium stearate;
   the second part is an extended-release layer, comprising:
about 30-45 mg of the compound of formula (I), or the pharmaceutically acceptable salt thereof;
about 42-63 mg of silicified microcrystalline cellulose;
about 35-60 mg of hypromellose K100M;
about 15-30 mg of sodium lauryl sulfate;
about 0.1-3 mg of colloidal silicone dioxide; and
about 0.1-5 mg of magnesium stearate.

In a specific embodiment, the pharmaceutical formulation is a bilayer tablet, the first part is an immediate-release layer, comprising:
about 49.2 mg of the compound of formula (I), or the pharmaceutically acceptable salt thereof;
about 51.8 mg of silicified microcrystalline cellulose;
about 3.4 mg of croscarmellose sodium;
about 4.0 mg of sodium lauryl sulfate;
about 0.6 mg of colloidal silicone dioxide; and
about 1.0 mg of magnesium stearate;
   the second part is an extended-release layer, comprising:
about 73.8 mg of the compound of formula (I), or the pharmaceutically acceptable salt thereof;
about 15.6 mg of silicified microcrystalline cellulose;
about 49.2 mg of Hypromellose K100M;
about 24 mg of sodium lauryl sulfate;
about 0.9 mg of colloidal silicone dioxide; and
about 1.5 mg of magnesium stearate.

In a specific embodiment, the pharmaceutical formulation is a bilayer tablet, the first part is an immediate-release layer, comprising:
about 49.2 mg of the compound of formula (I), or the pharmaceutically acceptable salt thereof;
about 51.8 mg of silicified microcrystalline cellulose;
about 3.4 mg of croscarmellose sodium;
about 4.0 mg of sodium lauryl sulfate;
about 0.6 mg of colloidal silicone dioxide; and
about 1.0 mg of magnesium stearate;
   the second part is an extended-release layer, comprising:
about 73.8 mg of the compound of formula (I), or the pharmaceutically acceptable salt thereof;
about 33.6 mg of silicified microcrystalline cellulose;
about 49.2 mg of hypromellose K100M;
about 6 mg of sodium lauryl sulfate;
about 0.9 mg of colloidal silicone dioxide; and
about 1.5 mg of magnesium stearate.

In a specific embodiment, the pharmaceutical formulation is a bilayer tablet, the first part is an immediate-release layer, comprising:
about 24.6 mg of the compound of formula (I), or the pharmaceutically acceptable salt thereof;
about 76.4 mg of silicified microcrystalline cellulose;
about 3.4 mg of croscarmellose sodium;
about 4.0 mg of sodium lauryl sulfate;
about 0.6 mg of colloidal silicone dioxide; and
about 1.0 mg of magnesium stearate;
   the second part is an extended-release layer, comprising:
about 36.9 mg of the compound of formula (I), or the pharmaceutically acceptable salt thereof;
about 52.5 mg of silicified microcrystalline cellulose;
about 49.2 mg of hypromellose K100M;
about 24.0 mg of sodium lauryl sulfate;
about 0.9 mg of colloidal silicone dioxide; and
about 1.5 mg of magnesium stearate.

In a specific embodiment, the pharmaceutical formulation is a bilayer tablet, the first part is an immediate-release layer, comprising:
about 24.60 mg of the compound of formula (I), or a corresponding amount of the pharmaceutically acceptable salt thereof;
about 98.70 mg of silicified microcrystalline cellulose;
about 4.17 mg of croscarmellose sodium;
about 4.91 mg of sodium lauryl sulfate;
about 0.74 mg of colloidal silicone dioxide; and
about 1.88 mg of magnesium stearate;
   the second part is an extended-release layer, comprising:
about 36.90 mg of the compound of formula (I) or a corresponding amount of the pharmaceutically acceptable salt thereof;
about 52.00 mg of silicified microcrystalline cellulose;
about 49.20 mg of hypromellose K100M;
about 24.00 mg of sodium lauryl sulfate;
about 0.90 mg of colloidal silicone dioxide; and
about 2.00 mg of magnesium stearate.

In a specific embodiment, the pharmaceutical formulation is a bilayer tablet, the first part is an immediate-release layer, comprising:
about 49.20 mg of the compound of formula (I), or a corresponding amount of the pharmaceutically acceptable salt thereof;
about 73.54 mg of silicified microcrystalline cellulose;
about 4.17 mg of croscarmellose sodium;
about 4.91 mg of sodium lauryl sulfate;
about 0.74 mg of colloidal silicone dioxide; and
about 2.44 mg of magnesium stearate;
   the second part is an extended-release layer, comprising:
about 73.80 mg of the compound of formula (I) or a corresponding amount of the pharmaceutically acceptable salt thereof;
about 14.62 mg of silicified microcrystalline cellulose;
about 49.20 mg of hypromellose K100M;
about 24.00 mg of sodium lauryl sulfate;
about 0.90 mg of colloidal silicone dioxide; and
about 2.48 mg of magnesium stearate.

In a specific embodiment, the pharmaceutical formulation is a bilayer tablet, the first part is an immediate-release layer, comprising:
about 49.20 mg of the compound of formula (I), or a corresponding amount of the pharmaceutically acceptable salt thereof;
about 69.88 mg of silicified microcrystalline cellulose;
about 4.20 mg of croscarmellose sodium;
about 9.60 mg of sodium lauryl sulfate;
about 0.72 mg of colloidal silicone dioxide; and
about 2.40 mg of magnesium stearate;
   the second part is an extended-release layer, comprising:
about 73.80 mg of the compound of formula (I) or a corresponding amount of the pharmaceutically acceptable salt thereof;
about 17.76 mg of silicified microcrystalline cellulose;
about 60.36 mg of hypromellose K100M;
about 48.00 mg of sodium lauryl sulfate;
about 1.08 mg of colloidal silicone dioxide; and
about 3.00 mg of magnesium stearate.

In a specific embodiment, the pharmaceutical formulation is a bilayer tablet, the first part is an immediate-release layer, comprising:
about 49.20 mg of the compound of formula (I), or a corresponding amount of the pharmaceutically acceptable salt thereof;
about 69.88 mg of silicified microcrystalline cellulose;
about 4.20 mg of croscarmellose sodium;
about 9.60 mg of sodium lauryl sulfate;
about 0.72 mg of colloidal silicone dioxide; and
about 2.40 mg of magnesium stearate;
   the second part is an extended-release layer, comprising:
about 73.80 mg of the compound of formula (I) or a corresponding amount of the pharmaceutically acceptable salt thereof;
about 17.76 mg of silicified microcrystalline cellulose;
about 60.36 mg of hypromellose K4M;
about 48.00 mg of sodium lauryl sulfate;
about 1.08 mg of colloidal silicone dioxide; and
about 3.00 mg of magnesium stearate.

In a specific embodiment, the pharmaceutical formulation is a bilayer tablet, the first part is an immediate-release layer, comprising:
about 73.80 mg of the compound of formula (I), or a corresponding amount of the pharmaceutically acceptable salt thereof;
about 104.82 mg of silicified microcrystalline cellulose;
about 6.30 mg of croscarmellose sodium;
about 14.40 mg of sodium lauryl sulfate;
about 1.08 mg of colloidal silicone dioxide; and
about 3.60 mg of magnesium stearate;
   the second part is an extended-release layer, comprising:
about 110.70 mg of the compound of formula (I) or a corresponding amount of the pharmaceutically acceptable salt thereof;
about 26.64 mg of silicified microcrystalline cellulose;
about 90.54 mg of hypromellose K100M;
about 72.00 mg of sodium lauryl sulfate;
about 1.62 mg of colloidal silicone dioxide; and
about 4.50 mg of magnesium stearate.

In a specific embodiment, the pharmaceutical formulation is a bilayer tablet, the first part is an immediate-release layer, comprising:
about 73.80 mg of the compound of formula (I), or a corresponding amount of the pharmaceutically acceptable salt thereof;
about 104.82 mg of silicified microcrystalline cellulose;
about 6.30 mg of croscarmellose sodium;
about 14.40 mg of sodium lauryl sulfate;
about 1.08 mg of colloidal silicone dioxide; and
about 3.60 mg of magnesium stearate;
   the second part is an extended-release layer, comprising:
about 110.70 mg of the compound of formula (I) or a corresponding amount of the pharmaceutically acceptable salt thereof;
about 26.64 mg of silicified microcrystalline cellulose;
about 90.54 mg of hypromellose K4M;
about 72.00 mg of sodium lauryl sulfate;
about 1.62 mg of colloidal silicone dioxide; and
about 4.50 mg of magnesium stearate.

In a specific embodiment, the pharmaceutical formulation is a bilayer tablet, the first part is an immediate-release layer, comprising: about 98.40 mg of the compound of formula (I), or a corresponding amount of the pharmaceutically acceptable salt thereof;
about 139.76 mg of silicified microcrystalline cellulose;
about 8.40 mg of croscarmellose sodium;
about 19.20 mg of sodium lauryl sulfate;
about 1.44 mg of colloidal silicone dioxide; and
about 4.80 mg of magnesium stearate;
   the second part is an extended-release layer, comprising:
about 147.60 mg of the compound of formula (I) or a corresponding amount of the pharmaceutically acceptable salt thereof;
about 35.52 mg of silicified microcrystalline cellulose;
about 120.72 mg of hypromellose K4M;
about 96.00 mg of sodium lauryl sulfate;
about 2.16 mg of colloidal silicone dioxide; and
about 6.00 mg of magnesium stearate.

In a specific embodiment, the pharmaceutical formulation is a bilayer tablet, the first part is an immediate-release layer, comprising: about 98.40 mg of the compound of formula (I), or a corresponding amount of the pharmaceutically acceptable salt thereof;
about 139.76 mg of silicified microcrystalline cellulose;
about 8.40 mg of croscarmellose sodium;
about 19.20 mg of sodium lauryl sulfate;
about 1.44 mg of colloidal silicone dioxide; and
about 4.80 mg of magnesium stearate;
   the second part is an extended-release layer, comprising:
about 147.60 mg of the compound of formula (I) or a corresponding amount of the pharmaceutically acceptable salt thereof;
about 35.52 mg of silicified microcrystalline cellulose;
about 120.72 mg of hypromellose K100LV;
about 96.00 mg of sodium lauryl sulfate;
about 2.16 mg of colloidal silicone dioxide; and
about 6.00 mg of magnesium stearate.

In a specific embodiment, the pharmaceutical formulation is a bilayer tablet, the first part is an immediate-release layer, comprising: about 123.00 mg of the compound of formula (I), or a corresponding amount of the pharmaceutically acceptable salt thereof;
about 174.70 mg of silicified microcrystalline cellulose;
about 10.50 mg of croscarmellose sodium;
about 24.00 mg of sodium lauryl sulfate;
about 1.80 mg of colloidal silicone dioxide; and
about 6.00 mg of magnesium stearate;
   the second part is an extended-release layer, comprising:
about 123.00 mg of the compound of formula (I) or a corresponding amount of the pharmaceutically acceptable salt thereof;
about 29.60 mg of silicified microcrystalline cellulose;
about 100.60 mg of hypromellose K100LV;
about 80.00 mg of sodium lauryl sulfate;
about 1.80 mg of colloidal silicone dioxide; and
about 5.00 mg of magnesium stearate.

In some embodiments, the pharmaceutical formulation is film-coated tablets.

In some embodiments, the film-coated bilayer tablet comprises coating agents selected from the group consisting of sodium carboxymethylcellulose, cellulose acetate, cellulose acetate phthalate, ethylcellulose, gelatin, pharmaceutical glaze, hydroxypropyl cellulose, hydroxypropylmethyl cellulose, hydroxypropyl methylcellulose phthalate, methacrylic acid copolymer, methylcellulose, polyethylene glycol, polyvinyl acetate phthalate, shellac, sucrose, titanium dioxide, carnauba wax, microcrystalline wax, zein, Polyvinyl Alcohol-Part. Hydrolyzed, Xanthan Gum, Gum Acacia, Macrogol (PEG) Polyvinyl Alcohol Graft Copolymer and Opadry.

In some embodiments, the coating agent is Opadry.

In some embodiments, the Opadry (such as Opadry II) is present in the amount of about 2-6% by weight of the core tablet.

In some embodiments, the pharmaceutical formulation has a dissolution rate of >20% at 1 hour. In some embodiments, the pharmaceutical formulation has a dissolution rate of >30% at 1 hour. In some embodiments, the pharmaceutical formulation has a dissolution rate of ≥40% at 1 hour.

In some embodiments, the pharmaceutical formulation is administered once-a-day.

### Method of Use

In some embodiments, the present disclosure provides a method of preparing single part formulations (single layer IR, ER tables, immediate-release part or extended-release part), and the steps of the method include:
(1) mixing all the ingredients, alternatively, mixing all the ingredients except the lubricant first;
(2) adding the lubricant to mix, alternatively further filling the obtained mixture into a vial, a sachet or a capsule, or compressing it into a tablet with a desired shape; and yet alternatively, further coating the obtained tablet.

In some other embodiments, the present disclosure provides a method of preparing single part formulations (single layer IR, ER tablets, immediate-release part or extended-release part), and the steps of the method include:
(1) mixing all the ingredients, alternatively, mixing all the ingredients for dry granulation such as slugging or roller compaction except retaining portion of glidant and lubricant;
(2) adding the glidant and lubricant to mix with the granulated and milled granules, alternatively further filling the obtained mixture into a vial, a sachet or a capsule, or compressing it into a tablet with a desired shape; and yet alternatively, further coating the obtained tablet.

In some embodiments, the present disclosure provides a method of preparing the bi-layer formulations, the first part and the second part perform the granulation and/or blending procedure as defined above separately, alternatively further filling the obtained two mixtures into a vial, a sachet or a capsule, or compressing it into a tablet with a desired shape; and yet alternatively, further coating the obtained tablet.

In some embodiments, the present disclosure provides the above pharmaceutical formulations in the manufacture of a medicament for preventing, slowing the progression of, delaying, or treating one or more metabolic disorders selected from the group consisting of: diabetes (including type I, type II diabetes, type II diabetes with obesity, and cystic fibrosis-related diabetes (CFRD)), diabetic kidney disease, impaired glucose tolerance, impaired fasting blood glucose, hyperglycemia, postprandial hyperglycemia, overweight, obesity, hypertension, insulin resistance, diseases associated with islet dysfunction and/or metabolic syndrome; or improving blood glucose control and/or reducing fasting plasma glucose, postprandial plasma glucose and/or glycosylated hemoglobin HbA1c; or preventing, slowing, delaying, or reversing complications of diabetes mellitus; or curing diabetes, causing diabetes remission or regressing diabetes.

The present disclosure also provides methods of treating diseases or disorders associated with GK activity comprising administering to a mammal, e.g., a human, in need of such treatment a therapeutically effective amount of the pharmaceutical formulation described herein, and optionally a film coated tablet. The pharmaceutical formulation of the present disclosure, as described herein, can be administered to a mammal, e.g., a human, for preventing, slowing the progression of, delaying, or treating one or more metabolic disorders selected from the group consisting of: diabetes including type I, type II diabetes, type II diabetes with obesity, and cystic fibrosis-related diabetes (CFRD)), diabetic kidney disease, impaired glucose tolerance, impaired fasting blood glucose, hyperglycemia, postprandial hyperglycemia, overweight, obesity, hypertension, insulin resistance, diseases associated with islet dysfunction and/or metabolic syndrome; or improving blood glucose control and/or reducing fasting plasma glucose, postprandial plasma glucose and/or glycosylated hemoglobin HbA1c; or preventing, slowing, delaying, or reversing complications of diabetes mellitus; or curing diabetes, causing diabetes remission or regressing diabetes.

In some embodiments, the pharmaceutical formulation of the present disclosure is administered to humans for preventing, slowing the progression of, delaying, or treating type II diabetes with obesity.

In some embodiments, the pharmaceutical formulation is administered orally.

In some embodiments, pharmaceutical formulation is administered orally as a tablet.

In some embodiments, pharmaceutical formulation is administered orally as a bilayer tablet.

In some embodiments, the pharmaceutical formulation is administered once-a-day.

In some embodiments, the therapeutically effective amount of the compound of formula (I) in the pharmaceutical formulation is about 30.75 mg, about 61.5 mg, about 92 mg, about 123 mg, about 184 mg, about 246 mg, about 307.5 mg, about 369 mg, about 430.5 mg, or about 492 mg per day. In some embodiments, the therapeutically effective amount of the compound of formula (I) in the pharmaceutical formulation is about 184.5 mg, about 185 mg.

### EXAMPLES

The present disclosure will be better understood by reference to the following Examples, which are provided as exemplary of the invention, and not by way of limitation.

The materials or reagents used herein are commercially available or prepared by synthetic methods commonly known in the art.

The following examples further describe and illustrate embodiments that are within the scope of the present disclosure. However, the present disclosure is not limited to the examples, and any modifications and replacements made on the technical basis of the present disclosure fall within the protective scope of the present disclosure.

### Example 1

Dorzagliatin (i.e. HMS5552) can be prepared using similar procedures as described in U.S. Pat. No. 7,741,327 B2.

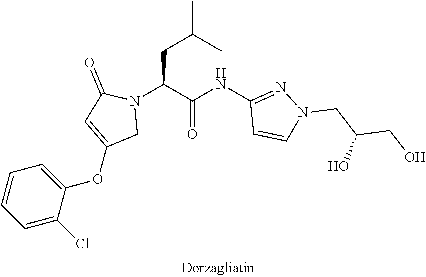 [SMILES: CC(C)C[C@@H](C(=O)Nc1ccn(C[C@@H](O)CO)n1)N1CC(Oc2ccccc2Cl)=CC1=O]

### Example 2

The compound of formula (I) can be prepared using similar procedures as described in WO 2023/040937A1.

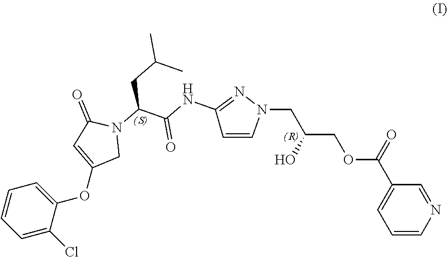

### Example 3

Single layer formulation IR1 comprising the compound of formula (I) (123.0 mg) as described in Table 1, which is an immediate-release formulation.

TABLE 1
Formulation of IR1 (Tablet A)

| Constituent | Function | Amount/mg | % w/w |
|---|---|---|---|
| The compound of formula (I) | API | 123.0 | 44.73 |
| Silicified Microcrystalline | Filler | 129.5 | 47.09 |
| Cellulose (ProSolv 90) | | | |
| Croscarmellose sodium, NF | Disintegrant | 8.5 | 3.09 |
| Sodium Lauryl Sulfate, NF (SLS) | Surfactant | 10.0 | 3.64 |
| Colloidal Silicone Dioxide | Glidant | 1.5 | 0.54 |
| (Aerosil 200) | | | |
| Magnesium Stearate, NF | Lubricant | 2.5 | 0.91 |

| | | |
|---|---|---|
| Total | 275.0 | 100.00 |

Single layer formulation IR2-IR7 comprising the compound of formula (I) as described in Table 2, which are immediate-release formulations, and can be used to prepare bilayer tablets of present disclosure.

TABLE 2
Formulation of IR2-IR7

| | IR2 | | IR3 | | IR4 | | IR5 | | IR6 | | IR7 | |
|---|---|---|---|---|---|---|---|---|---|---|---|---|
| | Amount | % | Amount | % | Amount | % | Amount | % | Amount | % | Amount | % |
| Formulation | (mg) | W/W | (mg) | W/W | (mg) | W/W | (mg) | W/W | (mg) | W/W | (mg) | W/W |
| API | 92.0 | 33.5 | 92.0 | 33.5 | 92.0 | 33.5 | 92.0 | 37.6 | 61.5 | 22.4 | 30.75 | 11.2 |
| Silicified | 171.7 | 62.4 | 161.7 | 58.8 | 141.7 | 51.5 | 131.7 | 53.8 | 191.0 | 69.5 | 221.75 | 80.6 |
| Microcrystalline | | | | | | | | | | | | |
| Cellulose | | | | | | | | | | | | |
| (ProSolv 90) | | | | | | | | | | | | |
| Croscarmellose | 8.5 | 3.1 | 8.5 | 3.1 | 8.5 | 3.1 | 8.5 | 3.5 | 8.5 | 3.1 | 8.5 | 3.1 |
| sodium, NF | | | | | | | | | | | | |
| Colloidal | 0.0 | 0.0 | 0.0 | 0.0 | 0.0 | 0.0 | 10.0 | 4.1 | 1.5 | 0.5 | 1.5 | 0.5 |
| Silicone Dioxide | | | | | | | | | | | | |
| (Aerosil 200) | | | | | | | | | | | | |
| Sodium Lauryl | 0.0 | 0.0 | 10.0 | 3.6 | 30.0 | 10.9 | 0.0 | 0.0 | 10.0 | 3.6 | 10.0 | 3.6 |
| Sulfate, NF(SLS) | | | | | | | | | | | | |
| Magnesium | 2.8 | 1.0 | 2.8 | 1.0 | 2.8 | 1.0 | 2.8 | 1.1 | 2.5 | 0.9 | 2.50 | 0.9 |
| Stearate, NF | | | | | | | | | | | | |
| Total | 275.0 | 100.0 | 275.0 | 100.0 | 275.0 | 100.0 | 245.0 | 100.00 | 275.0 | 100.0 | 275.0 | 100.0 |

Single layer formulation IR8-IR10 comprising the compound of formula (I) as described in Table 2-1, which are immediate-release formulations.

TABLE 2-1
Formulation of IR8-IR10

| | IR8 | | IR9 | | IR10 | |
|---|---|---|---|---|---|---|
| | Amount | % | Amount | % | Amount | % |
| Formulation | (mg) | W/W | (mg) | W/W | (mg) | W/W |
| The compound of | 61.50 | 18.22 | 123.00 | 36.44 | 123.00 | 36.18 |
| formula (I) | | | | | | |
| Silicified | 246.75 | 73.11 | 183.85 | 54.47 | 174.70 | 51.38 |
| Microcrystalline | | | | | | |
| Cellulose (ProSolv 90) | | | | | | |
| Croscarmellose | 10.42 | 3.09 | 10.42 | 3.09 | 10.50 | 3.09 |
| sodium, NF | | | | | | |
| Sodium Lauryl | 12.28 | 3.64 | 12.28 | 3.64 | 24.00 | 7.06 |
| Sulfate, NF (SLS) | | | | | | |
| Colloidal Silicone | 1.85 | 0.55 | 1.85 | 0.55 | 1.80 | 0.53 |
| Dioxide (Aerosil 200) | | | | | | |
| Magnesium Stearate, NF | 4.70 | 1.39 | 6.10 | 1.81 | 6.00 | 1.76 |
| Total | 337.50 | 100.00 | 337.50 | 100.00 | 340.00 | 100.00 |

In some embodiments, the single layer immediate-release formulations provided herein is used alone in the methods provided herein. In some embodiments, the single layer immediate-release formulations provided herein is used to prepare bilayer tablets provided herein.

Procedure for preparation of IR single layer tablets:
1) Mix API with all the excipients except the magnesium stearate in a mortar and pestle until uniform.
2) Add the magnesium stearate by geometric dilution and mix.
3) Compress single layer tablets using Carver Press with 9.53 mm FFBE round tooling.

### Example 4

Extended-release formulations (ER1, ER2 and ER3) of the compound of formula (I) were prepared as described in Table 3-5.

TABLE 3
Formulation of ER1 (Tablet B)
ER1

| Constituent | Function | Amount/mg | % W/W |
|---|---|---|---|
| The compound of formula (I) | API | 123.0 | 44.7 |
| Silicified Microcrystalline | Filler | 26.0 | 9.5 |
| Cellulose (ProSolv 90) | | | |
| Hypromellose K100LV | Release | 82.0 | 29.8 |
| | modifier | | |
| Sodium Lauryl Sulfate, NF (SLS) | Surfactant | 40.0 | 14.5 |
| Colloidal Silicone Dioxide | Glidant | 1.5 | 0.5 |
| (Aerosil 200) | | | |
| Magnesium Stearate, NF | Lubricant | 2.5 | 0.9 |

| | | |
|---|---|---|
| TOTAL | 275 | 100.0 |

TABLE 4
Formulation of ER2 (Tablet C)
ER2

| | | Amount/ | |
|---|---|---|---|
| Constituent | Function | mg | % W/W |
| The compound of formula (I) | API | 123.0 | 44.7 |
| Silicified Microcrystalline | Filler | 56.0 | 20.4 |
| Cellulose (ProSolv 90) | | | |
| Hypromellose K100M | Release modifier | 82.0 | 29.8 |
| Sodium Lauryl Sulfate, NF | Surfactant | 10.0 | 3.6 |
| (SLS) | | | |
| Colloidal Silicone Dioxide | Glidant | 1.5 | 0.5 |
| (Aerosil 200) | | | |
| Magnesium Stearate, NF | Lubricant | 2.5 | 0.9 |

| | | |
|---|---|---|
| TOTAL | 275 | 100.0 |

TABLE 5
Formulation of ER3
ER3

| | | Amount/ | |
|---|---|---|---|
| Constituent | Function | mg | % W/W |
| The compound of formula (I) | API | 123.0 | 44.7 |
| Silicified Microcrystalline | Filler | 26.0 | 9.5 |
| Cellulose (ProSolv 90) | | | |
| Hypromellose K100M | Release modifier | 82.0 | 29.8 |
| Sodium Lauryl Sulfate, NF | Surfactant | 40.0 | 14.5 |
| (SLS) | | | |
| Colloidal Silicone Dioxide | Glidant | 1.5 | 0.5 |
| (Aerosil 200) | | | |
| Magnesium Stearate, NF | Lubricant | 2.5 | 0.9 |

| | | |
|---|---|---|
| TOTAL | 275.0 | 100.0 |

Extended-release formulations ER4-ER8 comprising the compound of formula (I) were prepared as described in Table 6.

TABLE 6
Formulations of ER4-ER8

| | ER4 | | ER5 | | ER6 | | ER7 | | ER8 | |
|---|---|---|---|---|---|---|---|---|---|---|
| | Amount | % | Amount | % | Amount | % | Amount | % | Amount | % |
| Constituent | (mg) | W/W | (mg) | W/W | (mg) | W/W | (mg) | W/W | (mg) | W/W |
| The compound of | 123.0 | 44.7 | 123.0 | 44.7 | 123.0 | 44.7 | 184.0 | 46.0 | 184.0 | 46.0 |
| formula (I) | | | | | | | | | | |
| Silicified | 26.0 | 9.5 | 26.0 | 9.5 | 66.0 | 24.0 | 32.0 | 8.0 | 0.0 | 0.0 |
| Microcrystalline | | | | | | | | | | |
| Cellulose (ProSolv 90) | | | | | | | | | | |
| Hypromellose E5 | 82.0 | 29.8 | 41.0 | 14.9 | 0.0 | 0.0 | 120.0 | 30.0 | 120.0 | 30.0 |
| Hypromellose K100LV | 0.0 | 0.0 | 41.0 | 14.9 | 0.0 | 0.0 | 0.0 | 0.0 | 0.0 | 0.0 |
| Hypromellose K100M | 0.0 | 0.0 | 0.0 | 0.0 | 82.0 | 29.8 | 0.0 | 0.0 | 0.0 | 0.0 |
| Colloidal Silicone | 1.5 | 0.5 | 1.5 | 0.5 | 1.5 | 0.5 | 0.0 | 0.0 | 0.0 | 0.0 |
| Dioxide (Aerosil 200) | | | | | | | | | | |
| Sodium Lauryl | 40.0 | 14.5 | 40.0 | 14.5 | 0.0 | 0.0 | 60.0 | 15.0 | 92.0 | 23.0 |
| Sulfate, NF(SLS) | | | | | | | | | | |
| Magnesium Stearate, | 2.5 | 0.9 | 2.5 | 0.9 | 2.5 | 0.9 | 4.0 | 1.0 | 4.0 | 1.0 |
| NF | | | | | | | | | | |
| Total | 275.0 | 100.0 | 275.0 | 100.0 | 275.0 | 100.0 | 400.0 | 100.0 | 400.0 | 100.0 |

Extended-release formulations ER9-ER12 comprising the compound of formula (I) were prepared as described in Table 6-1.

TABLE 6-1
Formulations of ER9-ER12

| | ER9 | | ER10 | | ER11 | | ER12 | |
|---|---|---|---|---|---|---|---|---|
| | Amount | % | Amount | % | Amount | % | Amount | % |
| Constituent | (mg) | W/W | (mg) | W/W | (mg) | W/W | (mg) | W/W |
| The compound of | 61.50 | 22.36 | 123.00 | 44.73 | 123.00 | 36.18 | 123.00 | 36.18 |
| formula (I) | | | | | | | | |
| Silicified | 86.67 | 31.52 | 24.37 | 8.86 | 29.60 | 8.71 | 29.60 | 8.71 |
| Microcrystalline | | | | | | | | |
| Cellulose (ProSolv 90) | | | | | | | | |
| Hypromellose K100M | 82.00 | 29.82 | 82.00 | 29.82 | 100.60 | 29.59 | 0.00 | 0.00 |
| Hypromellose K4M | 0.00 | 0.00 | 0.00 | 0.00 | 0.00 | 0.00 | 100.60 | 29.59 |
| Sodium Lauryl Sulfate, | 40.00 | 14.55 | 40.00 | 14.55 | 80.00 | 23.53 | 80.00 | 23.53 |
| NF (SLS) | | | | | | | | |
| Colloidal Silicone Dioxide | 1.50 | 0.55 | 1.50 | 0.55 | 1.80 | 0.53 | 1.80 | 0.53 |
| (Aerosil 200) | | | | | | | | |
| Magnesium Stearate, NF | 3.33 | 1.21 | 4.13 | 1.50 | 5.00 | 1.47 | 5.00 | 1.47 |
| Total | 275.00 | 100.0 | 275.00 | 100.00 | 340.00 | 100.00 | 340.00 | 100.00 |

In some embodiments, the single layer extended-release formulations provided herein is used alone in the methods provided herein. In some embodiments, the single layer extended-release formulations provided herein is used to prepare bilayer tablets provided herein.

### Procedure for Preparation of ER Single Layer Tablets:

1) Mix API with all the excipients except the magnesium stearate in a mortar and pestle until uniform.
2) Add the magnesium stearate by geometric dilution and mix.
3) Compress single layer tablets using Carver Press with 9.53 mm FFBE round tooling.

### Example 5

A bilayer tablet D of the compound of formula (I) (123 mg) was prepared as described below, which comprises an immediate-release layer and an extended-release layer.

TABLE 7
Formulation of bilayer tablet D with 123 mg strength

| | | | |
|---|---|---|---|
| | | Content/ | % |
| Constituent | Function | mg | (w/w) |

Immediate-release layer

| | | | |
|---|---|---|---|
| The compound of formula (I) | API | 49.2 | 44.73 |
| Silicified Microcrystalline | Filler | 51.8 | 47.09 |
| Cellulose (ProSolv 90) | | | |
| Croscarmellose sodium, NF | Disintegrant | 3.4 | 3.09 |
| Sodium Lauryl Sulfate, NF | Surfactant | 4.0 | 3.64 |
| (SLS) | | | |
| Colloidal Silicone Dioxide | Glidant | 0.6 | 0.54 |
| (Aerosil 200) | | | |
| Magnesium Stearate, NF | Lubricant | 1.0 | 0.91 |

| | | |
|---|---|---|
| Subtotal | 110 | 100.00 |

Extended-release layer

| | | | |
|---|---|---|---|
| The compound of formula (I) | API | 73.8 | 44.73 |
| Silicified Microcrystalline | Filler | 15.6 | 9.45 |
| Cellulose (ProSolv 90) | | | |
| Hypromellose K100M | Release modifier | 49.2 | 29.82 |
| Sodium Lauryl Sulfate, NF | Surfactant | 24.0 | 14.55 |
| (SLS) | | | |
| Colloidal Silicone Dioxide | Glidant | 0.9 | 0.54 |
| (Aerosil 200) | | | |
| Magnesium Stearate, NF | Lubricant | 1.5 | 0.91 |

| | | |
|---|---|---|
| Subtotal | 165.0 | 100.00 |
| Total | 275.0 | — |

### Procedure for Preparation of Bilayer Tablets:

Same blending procedure for both TR and ER layer blend, performed separately.
1) Mixing API with all the excipients except the magnesium stearate in a mortar and pestle until uniform.
2) Adding the magnesium stearate by geometric dilution and mix.
3) Compressing bilayer tablets (slug the first layer blend then adding the second layer blend and compress again) using Carver Press with 9.53 mm FFBE round tooling.

### Example 6

A bilayer tablet E of the compound of formula (I) (123 mg) was prepared in the same procedure as described in Example 5, which comprises an immediate-release layer and an extended-release layer.

TABLE 8
Formulation of bilayer tablet E with 123 mg strength

| | | | |
|---|---|---|---|
| | | | % |
| Constituent | Function | Content/mg | (w/w) |

Immediate-release layer

| | | | |
|---|---|---|---|
| The compound of formula (I) | API | 49.2 | 44.73 |
| Silicified Microcrystalline | Filler | 51.8 | 47.09 |
| Cellulose (ProSolv 90) | | | |
| Croscarmellose sodium, NF | Disintegrant | 3.4 | 3.09 |
| Sodium Lauryl Sulfate, NF (SLS) | Surfactant | 4.0 | 3.64 |
| Colloidal Silicone Dioxide | Glidant | 0.6 | 0.54 |
| (Aerosil 200) | | | |
| Magnesium Stearate, NF | Lubricant | 1.0 | 0.91 |

| | | |
|---|---|---|
| Subtotal | 110 | 100.00 |

Extended-release layer

| | | | |
|---|---|---|---|
| The compound of formula (I) | API | 73.8 | 44.73 |
| Silicified Microcrystalline | Filler | 33.6 | 20.36 |
| Cellulose (ProSolv 90) | | | |
| Hypromellose K100M | Release | 49.2 | 29.82 |
| | modifier | | |
| Sodium Lauryl Sulfate, NF (SLS) | Surfactant | 6.0 | 3.64 |
| Colloidal Silicone Dioxide | Glidant | 0.9 | 0.54 |
| (Aerosil 200) | | | |
| Magnesium Stearate, NF | Lubricant | 1.5 | 0.91 |

| | | |
|---|---|---|
| Subtotal | 165 | 100.00 |
| Total | 275 | — |

### Example 7

A bilayer tablet F of the compound of formula (I) (61.5 mg) was prepared in the same procedure as described in Example 5, which comprises an immediate-release layer and an extended-release layer.

TABLE 9
Formulation of bilayer tablet with 61.5 mg strength

| | | | |
|---|---|---|---|
| | | | % |
| Constituent | Function | Content/mg | (w/w) |

Immediate-release layer

| | | | |
|---|---|---|---|
| The compound of formula (I) | API | 24.6 | 22.36 |
| Silicified Microcrystalline | Filler | 76.4 | 69.45 |
| Cellulose (ProSolv 90) | | | |
| Croscarmellose sodium, NF | Disintegrant | 3.4 | 3.09 |
| Colloidal Silicone Dioxide | Glidant | 0.6 | 0.54 |
| (Aerosil 200) | | | |
| Sodium Lauryl Sulfate, | Surfactant | 4.0 | 3.64 |
| NF (SLS) | | | |
| Magnesium Stearate, NF | Lubricant | 1.0 | 0.91 |

| | | |
|---|---|---|
| Subtotal | 110.0 | 100.0 |

Extended-release layer

| | | | |
|---|---|---|---|
| The compound of formula (I) | API | 36.9 | 22.36 |
| Silicified Microcrystalline | Filler | 52.5 | 31.82 |
| Cellulose (ProSolv 90) | | | |
| Hypromellose K100M | Release | 49.2 | 29.82 |
| | modifier | | |
| Colloidal Silicone Dioxide | Glidant | 0.9 | 0.54 |
| (Aerosil 200) | | | |
| Sodium Lauryl Sulfate, | Surfactant | 24.0 | 14.55 |
| NF (SLS) | | | |
| Magnesium Stearate, NF | Lubricant | 1.5 | 0.91 |

| | | |
|---|---|---|
| Subtotal | 165.0 | 100.0 |
| Total | 275.0 | — |

A bilayer tablet G of the compound of formula (I) (61.5 mg) and a bilayer tablet H of the compound of formula (I) (123 mg) were prepared in the same procedure as described in Example 5, which comprises an immediate-release layer and an extended-release layer.

TABLE 9-1
Bilayer tablets G (61.5 mg) and H (123 mg)

| | | G (61.5 mg) | | H (123 mg) | |
|---|---|---|---|---|---|
| Constituent | Function | Content/mg | % (w/w) | Content/mg | % (w/w) |

Immediate-release layer

| | | | | | |
|---|---|---|---|---|---|
| The compound of formula (I) | API | 24.60 | 18.22 | 49.20 | 36.44 |
| Silicified Microcrystalline Cellulose | Filler | 98.70 | 73.11 | 73.54 | 54.47 |
| (ProSolv 90) | | | | | |
| Croscarmellose sodium, NF | Disintegrant | 4.17 | 3.09 | 4.17 | 3.09 |
| Sodium Lauryl Sulfate, NF (SLS) | Surfactant | 4.91 | 3.64 | 4.91 | 3.64 |
| Colloidal Silicone Dioxide (Aerosil 200) | Glidant | 0.74 | 0.55 | 0.74 | 0.55 |
| Magnesium Stearate, NF | Lubricant | 1.88 | 1.39 | 2.44 | 1.81 |

| | | | | |
|---|---|---|---|---|
| Subtotal | 135.00 | 100.00 | 135.00 | 100.00 |

Extended-release layer

| | | | | | |
|---|---|---|---|---|---|
| The compound of formula (I) | API | 36.90 | 22.36 | 73.80 | 44.73 |
| Silicified Microcrystalline Cellulose | Filler | 52.00 | 31.52 | 14.62 | 8.86 |
| (ProSolv 90) | | | | | |
| Hypromellose K100M | Release modifier | 49.20 | 29.82 | 49.20 | 29.82 |
| Sodium Lauryl Sulfate, NF (SLS) | Surfactant | 24.00 | 14.55 | 24.00 | 14.55 |
| Colloidal Silicone Dioxide (Aerosil 200) | Glidant | 0.90 | 0.55 | 0.90 | 0.55 |
| Magnesium Stearate, NF | Lubricant | 2.00 | 1.21 | 2.48 | 1.50 |

| | | | | |
|---|---|---|---|---|
| Subtotal | 165.00 | 100.00 | 165.00 | 100.00 |
| Total | 300.00 | — | 300.00 | — |

Bilayer tablets I and J of the compound of formula (I) (123 mg), bilayer tablets K1 and L of the compound of formula (I) (184.5 mg), and bilayer tablet M of the compound of formula (I) (246 mg) were prepared in the same procedure as described in Example 5, which comprises an immediate-release layer and an extended-release layer.

TABLE 9-2
Formulation of bilayer tablets I, J, K1, L, and M

| | I (123 mg) | | J (123 mg) | | K1 (184.5 mg) | | L (184.5 mg) | | M (246 mg) | |
|---|---|---|---|---|---|---|---|---|---|---|
| | Content/ | % | Content/ | % | Content/ | % | Content/ | % | Content/ | % |
| Constituent | mg | (w/w) | mg | (w/w) | mg | (w/w) | mg | (w/w) | mg | (w/w) |

Immediate-release layer

| | | | | | | | | | | |
|---|---|---|---|---|---|---|---|---|---|---|
| The compound | 49.20 | 36.18 | 49.20 | 36.18 | 73.80 | 36.18 | 73.80 | 36.18 | 98.40 | 36.18 |
| of formula (I) | | | | | | | | | | |
| Silicified | 69.88 | 51.38 | 69.88 | 51.38 | 104.82 | 51.38 | 104.82 | 51.38 | 139.76 | 51.38 |
| Microcrystalline | | | | | | | | | | |
| Cellulose | | | | | | | | | | |
| (ProSolv 90) | | | | | | | | | | |
| Croscarmellose | 4.20 | 3.09 | 4.20 | 3.09 | 6.30 | 3.09 | 6.30 | 3.09 | 8.40 | 3.09 |
| sodium, NF | | | | | | | | | | |
| Sodium Lauryl | 9.60 | 7.06 | 9.60 | 7.06 | 14.40 | 7.06 | 14.40 | 7.06 | 19.20 | 7.06 |
| Sulfate, NF (SLS) | | | | | | | | | | |
| Colloidal Silicone | 0.72 | 0.53 | 0.72 | 0.53 | 1.08 | 0.53 | 1.08 | 0.53 | 1.44 | 0.53 |
| Dioxide (Aerosil 200) | | | | | | | | | | |
| Magnesium | 2.40 | 1.76 | 2.40 | 1.76 | 3.60 | 1.76 | 3.60 | 1.76 | 4.8 | 1.76 |
| Stearate, NF | | | | | | | | | | |
| Subtotal | 136.00 | 100.00 | 136.00 | 100.00 | 204.00 | 100.00 | 204.00 | 100.00 | 272.00 | 100.00 |

Extended-release layer

| | | | | | | | | | | |
|---|---|---|---|---|---|---|---|---|---|---|
| The compound | 73.80 | 36.18 | 73.80 | 36.18 | 110.70 | 36.18 | 110.70 | 36.18 | 147.60 | 36.18 |
| of formula (I) | | | | | | | | | | |
| Silicified | 17.76 | 8.71 | 17.76 | 8.71 | 26.64 | 8.71 | 26.64 | 8.71 | 35.52 | 8.71 |
| Microcrystalline | | | | | | | | | | |
| Cellulose | | | | | | | | | | |
| (ProSolv 90) | | | | | | | | | | |
| Hypromellose | 60.36 | 29.59 | — | — | 90.54 | 29.59 | — | — | — | — |
| K100M | | | | | | | | | | |
| Hypromellose | — | — | 60.36 | 29.59 | — | — | 90.54 | 29.59 | 120.72 | 29.59 |
| K4M | | | | | | | | | | |
| Sodium Lauryl | 48.00 | 23.53 | 48.00 | 23.53 | 72.00 | 23.53 | 72.00 | 23.53 | 96.00 | 23.53 |
| Sulfate, NF | | | | | | | | | | |
| (SLS) | | | | | | | | | | |
| Colloidal | 1.08 | 0.53 | 1.08 | 0.53 | 1.62 | 0.53 | 1.62 | 0.53 | 2.16 | 0.53 |
| Silicone | | | | | | | | | | |
| Dioxide | | | | | | | | | | |
| (Aerosil 200) | | | | | | | | | | |
| Magnesium | 3.0 | 1.47 | 3.0 | 1.47 | 4.50 | 1.47 | 4.50 | 1.47 | 6.00 | 1.47 |
| Stearate, NF | | | | | | | | | | |
| Subtotal | 204.00 | 100.00 | 204.00 | 100.00 | 306.00 | 100.00 | 306.00 | 100.00 | 408.00 | 100.00 |
| Total | 340.00 | — | 340.00 | — | 510.00 | — | 510.00 | — | 680.00 | — |

Bilayer tablets N and O of the compound of formula (I) (246 mg) were prepared in the same procedure as described in Example 5, which comprises an immediate-release layer and an extended-release layer.

TABLE 9-3
Formulation of bilayer tablets N and O

| | N (246 mg) | | O (246 mg) | |
|---|---|---|---|---|
| Ingredients | mg/tab | Layer % | mg/tab | Layer % |

IR Layer formulas

| | | | | |
|---|---|---|---|---|
| Dorzagliatin Prodrug | 98.40 | 36.18% | 123.00 | 36.18% |
| Silicified Microcrystalline Cellulose | 139.76 | 51.38% | 174.70 | 51.38% |
| (ProSolv 90) | | | | |
| Croscarmellose sodium, NF | 8.40 | 3.09% | 10.50 | 3.09% |
| Sodium Lauryl Sulfate, NF (SLS) | 19.20 | 7.06% | 24.00 | 7.06% |
| Colloidal Silicone Dioxide | 1.44 | 0.53% | 1.80 | 0.53% |
| (Aerosil 200) | | | | |
| Magnesium Stearate, NF | 4.80 | 1.76% | 6.00 | 1.76% |
| Total | 272.00 | 100.00% | 340.00 | 100.00% |

ER Layer Formulas

| | | | | |
|---|---|---|---|---|
| Dorzagliatin Prodrug | 147.60 | 36.18% | 123.00 | 36.18% |
| Silicified Microcrystalline | 35.52 | 8.71% | 29.60 | 8.71% |
| Cellulose (ProSolv 90) | | | | |
| Hypromellose K100LV | 120.72 | 29.59% | 100.60 | 29.59% |
| Sodium Lauryl Sulfate, NF (SLS) | 96.00 | 23.53% | 80.00 | 23.53% |
| Colloidal Silicone Dioxide | 2.16 | 0.53% | 1.80 | 0.53% |
| (Aerosil 200) | | | | |
| Magnesium Stearate, NF | 6.00 | 1.47% | 5.00 | 1.47% |
| Total | 408.00 | 100.00% | 340.00 | 100.00% |
| Core Total | 680.00 | — | 680.00 | — |

### Example 8

### Preparation of Film Coated Bilayer Tablets

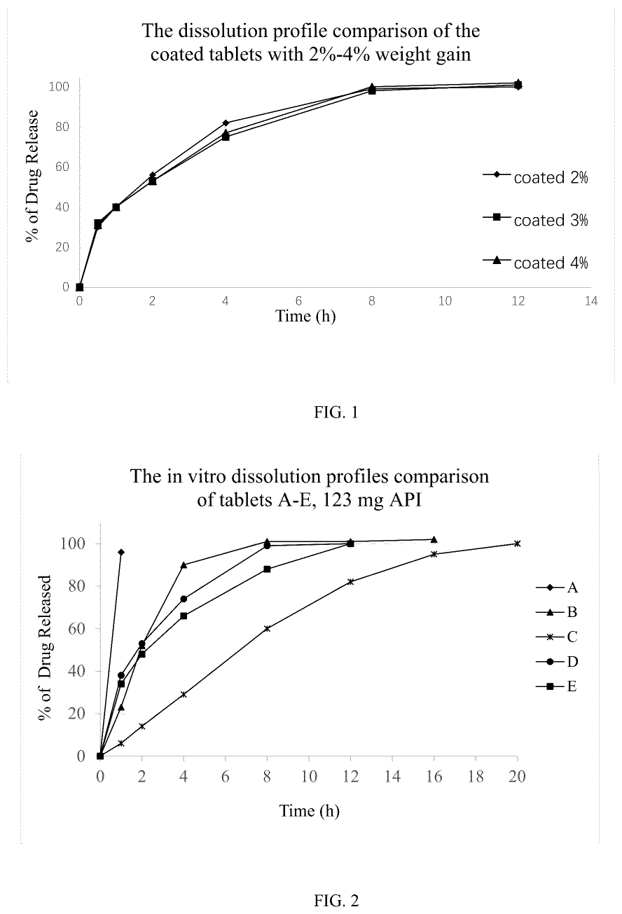

The core tablets prepared with formula from Example 5 were film coated in a BG 1-5 Film Pan Coater with Opadry® II Complete Film Coating System. The coating material was dispersed into the required amount of purified water with the aid of a propeller mixer and mixed for NLT 45 minutes. The coating dispersion was sprayed onto the core tablets with the appropriate coating parameters with tablet weight gain up to 2%, 3% and 4%. Different coating weight gain samples were tested for dissolution in 900 mL purified water of 0.5% SDS stirred at a paddle speed of 50 rpm. Dissolution profiles from different weight gain samples showed to be similar. The dissolution profiles are shown in FIG. 1.

### Example 9

### Tablets Dissolution Test

Samples from IR tablets, ER tablets, bilayer tablet D, E, and F were tested for dissolution in 900 mL purified water of 0.25% or 0.5% SDS stirred at a paddle speed of 50 rpm.

Samples from bilayer tablets G-O were tested for dissolution in 900 mL purified water of 0.5% SDS stirred at a paddle speed of 50 rpm.

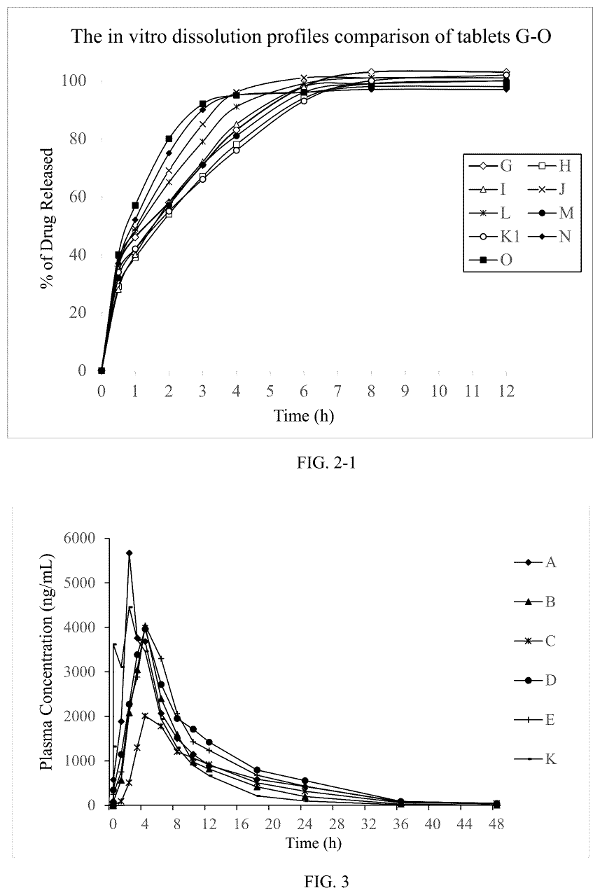

The dissolution results from the above tablets were summarized and shown in Tables 10-12, Table 12-1. The dissolution profiles of tablets A-E are shown in FIG. 2, and the dissolution profiles of tablets G-O are shown in FIG. 2-1.

TABLE 10
Dissolution profiles of tablets IR1-IR5

| | Cumulative drug release (%) | | | | |
|---|---|---|---|---|---|
| Time (min) | IR1 | IR2 | IR3* | IR4* | IR5* |
| 15 | 65 | 75 | 51 | 37 | 50 |
| 30 | 86 | 91 | 61 | 63 | 76 |
| 45 | 93 | 98 | 66 | 77 | 85 |
| 60 | 96 | 101 | 96 | 98 | 97 |

*Dissolution testing was performed in 0.25% of SDS.

TABLE 11
Dissolution profiles of tablets ER1-ER8

| Time | Cumulative drug release (%) | | | | | | | |
|---|---|---|---|---|---|---|---|---|
| (h) | ER1 | ER2 | ER3 | ER4 | ER5 | ER6 | ER7 | ER8 |
| 1 | 23 | 6 | 7 | 52 | 36 | 4 | 34 | 60 |
| 2 | 52 | 14 | 17 | 88 | 71 | 11 | 69 | 93 |
| 4 | 90 | 29 | 38 | 99 | 96 | 27 | 93 | 99 |
| 8 | 101 | 60 | 79 | 99 | 99 | 56 | 99 | 102 |
| 12 | 101 | 82 | 101 | — | — | 79 | 100 | 102 |
| 16 | 102 | 95 | 102 | — | — | 92 | 101 | 103 |
| 20 | — | 100 | — | — | — | 99 | 102 | — |

TABLE 12
Dissolution profiles of single layer IR tablet (tablet A), single layer
ER tablets (tablet B and tablet C) and bilayer tablets D, E, and F

| | |
|---|---|
| Time | Cumulative drug release (%) |

| (h) | A | B | C | D | E | F |
|---|---|---|---|---|---|---|
| 0 | 0 | 0 | 0 | 0 | 0 | 0 |
| 1 | 96 | 23 | 6 | 38 | 34 | 40 |
| 2 | — | 52 | 14 | 53 | 48 | 55 |
| 4 | — | 90 | 29 | 74 | 66 | 79 |
| 8 | — | 101 | 60 | 99 | 88 | 100 |
| 12 | — | 101 | 82 | 100 | 100 | 101 |
| 16 | — | 102 | 95 | — | — | — |
| 20 | — | — | 100 | — | — | — |

TABLE 12-1
Dissolution Profiles Comparison for bilayer tablets G to O
in 900 mL of 0.5% SDS stirred at a paddle speed of 50 rpm

| Time | Cumulative drug release (%) | | | | | | | | |
|---|---|---|---|---|---|---|---|---|---|
| (h) | G | H | I | J | K1 | L | M | N | O |
| 0 | 0 | 0 | 0 | 0 | 0 | 0 | 0 | 0 | 0 |
| 0.5 | 36 | 29 | 28 | 36 | 34 | 38 | 32 | 37 | 40 |
| 1 | 46 | 39 | 40 | 49 | 42 | 48 | 42 | 52 | 57 |
| 2 | 58 | 54 | 58 | 69 | 55 | 65 | 57 | 75 | 80 |
| 3 | 71 | 67 | 72 | 85 | 66 | 79 | 71 | 90 | 92 |
| 4 | 83 | 78 | 85 | 96 | 76 | 91 | 81 | 95 | 95 |
| 6 | 98 | 94 | 98 | 101 | 93 | 99 | 96 | 96 | 96 |
| 8 | 103 | 99 | 99 | 101 | 100 | 101 | 99 | 97 | 98 |
| 12 | 103 | 100 | 100 | 101 | 102 | 101 | 100 | 97 | 98 |

As shown in Table 10, IR tablets released more than about 85% at 1 hour after initiation of dissolution testing.

As shown in Table 11 and Table 12 and FIG. 2, all tablets had completely released the API at the end of the dissolution testing (tablet A released 98% after 75 minutes of dissolution).

Bilayer tablets D and E completely released all the API at 12 hours of dissolution time point.

As shown in Table 12-1 and FIG. 2-1, all bilayer tablets G-O had completely released the API at 12 hours of dissolution time point. Comparison of bilayer tablets N (246 mg) and O (246 mg) to tablet M (246 mg) was made using low viscosity polymer Hypromellose K100LV.

### Example 10

### Pharmacological and Pharmacokinetic Study

The purpose of this test example was to evaluate the pharmacokinetic/pharmacodynamic properties of tablets A-E and Dorzagliatin commercial products (HuaTangNing, 75 mg), identified as tablet K, following single oral (PO) administration in non-naïve male beagle dogs.

Eighteen non-naïve male beagle dogs supplied by Marshall Bioresources (Beijing and Jiangsu, China) were randomly divided into seven groups with 3 animals/group.

Fresh drinking water (reverses osmosis) was available to all animals, ad libitum. Animals were fed twice daily during non-study period with approximately 220 grams of certified animal diet daily from certified vendor. All animals were fed as usual prior to dosing and fed 110 g food on the morning of dosing day, then administered a single dose 4 hours later.

Animals in Groups 1 to 5 were administered tablets A-E by single oral administration at 123 mg/animal (equal to 100 mg of Dorzagliatin, also labeled as HMS5552-K in the following tables), respectively. Animals in Group 6 were administered tablet K by single oral administration at 75 mg/animal.

Approximately 0.5 mL blood samples were collected at pre-dose (0), 0.25, 0.5, 1, 2, 3, 4, 6, 8, 10, 12, 18, 24, 36 and 48 hours post-dose via peripheral vessel from each study animal. All blood samples were transferred into commercial tubes containing anticoagulant K2-EDTA (0.85-1.15 mg), placed on wet ice. Appropriate amount of blood was taken out to measure blood glucose with Accu-Chek Performa Glucose Meter and recorded. Plasma samples were prepared within 30 min of collection by centrifuging the blood samples at approximately 2-8° C., 3200×g for 10 minutes. Plasma samples were transferred to labeled polypropylene tubes and quickly frozen over dry ice and kept at −60° C. or lower until liquid chromatography tandem mass spectrometry (LC-MS/MS) analysis.

### Pharmacokinetics Data Analysis

The plasma concentration of HMS5552-K of tablet K and the compound of formula (I) in study animals was subjected to a non-compartmental pharmacokinetic analysis by using the Phoenix WinNonlin software (version 8.3.5, Certara). The linear/log trapezoidal rule was applied in obtaining the PK parameters. Mean plasma PK parameters of HMS5552-K are shown in Table 13, and FIG. 3.

TABLE 13
PK Parameters of HMS5552-K in Dogs

| | Treatment | | | | | |
|---|---|---|---|---|---|---|
| HMS5552-K | (123 mg/Tablet/Dog) | | | | | (75 mg/Tablet/Dog) |
| PK parameters | A | B | C | D | E | K |
| Cₘₐₓ (ng/mL) | 5667 | 4027 | 2173 | 3967 | 4043 | 4880 |
| Tₘₐₓ (h) | 2.00 | 4.00 | 4.67 | 3.67 | 4.00 | 1.67 |
| T_{1/2} (h) | 7.49 | 5.06 | 7.06 | 6.96 | 6.36 | 4.68 |
| AUC₀₋₄₈ (ng · h/mL) | 37299 | 28748 | 22899 | 40926 | 38377 | 30001 |
| AUC_{0-inf} (ng · h/mL) | 37779 | 28806 | 23364 | 41404 | 38746 | 30053 |
| MRT₀₋ₗₐₛₜ (h) | 9.43 | 8.68 | 11.7 | 11.1 | 10.2 | 6.25 |
| MRT_{0-inf} (h) | 10.1 | 8.78 | 12.5 | 11.6 | 10.7 | 6.33 |
| Cₘₐₓ Ratio (vs Group K) * | 0.87 | 0.62 | 0.33 | 0.61 | 0.62 | — |
| AUC₀₋₄₈ Ratio (vs Group K) * | 0.93 | 0.72 | 0.57 | 1.02 | 0.96 | — |
| AUC_{inf} Ratio (vs Group K) * | 0.94 | 0.72 | 0.58 | 1.03 | 0.97 | — |

* Cₘₐₓ and AUC values were normalized to the strength of HMS5552-75 mg.

As shown in Table 13, the relative bioavailability of HMS5552-K in tablets A, D, E relative to the tablet K of the parent drug were 94%, 103%, and 97%. IR tablet A, bilayer tablet D and E had lower Cₘₐₓ of HMS5552 (about ˜87%, 61%, 62% respectively) than tablet K. Bilayer tablet D and E had lower Cₘₐₓ of HMS5552 than IR tablet A. Lower Cₘₐₓ values usually reduce sides effects of medications.

The mean residence time (MRT) of HMS5552 in tablets A, D, E in dog plasma was nearly doubled compared to a single dose of HMS5552-K in tablet K, implicating the MRT of HMS5552 in single dose of A, D, or E is similar to the total MRT of HMS5552-K in tablet K BID.

According to T_{1/2}(h) in Table 13, the half-life of HMS5552-K was increased compared to a single dose of HMS5552-K in tablet K.

Increased MRT and T_{1/2}(h) for the compound from the extended-release formulations implicated longer absorption window resulted from longer GI track residence time and potentially increases greater stimulation of secretion of GLP-1.

Groups administered with IR tablet A, bilayer tablet D and E all had excellent (about 100%) relative bioavailability of HMS5552-K to tablet K group and much lower Cₘₐₓ of HMS5552-K than tablet K.

No safety issues were observed in this single dose study.

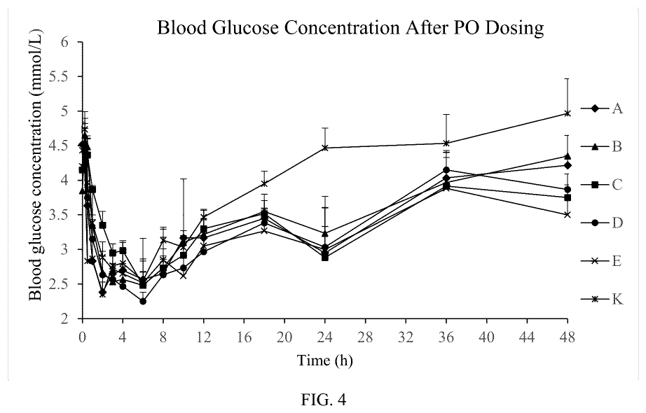

Mean blood glucose at each time point after administration is shown in Table 14 and the blood glucose-time curve is shown in FIG. 4.

Blood Glucose data after oral administration of different tablets are shown in Table 15.

TABLE 14
Mean blood glucose of each time points from tablets A-E and K

Mean BG in groups (mmol/L)

| Time(h) | A | B | C | D | E | K |
|---|---|---|---|---|---|---|
| 0 | 4.52 | 3.85 | 4.15 | 4.15 | 4.20 | 4.43 |
| 0.25 | 4.22 | 4.65 | 4.43 | 4.52 | 4.73 | 4.15 |
| 0.5 | 3.63 | 4.48 | 4.37 | 3.75 | 3.97 | 2.83 |
| 1 | 2.83 | 3.35 | 3.87 | 3.15 | 3.38 | 2.87 |
| 2 | 2.38 | 2.67 | 3.35 | 2.63 | 2.88 | 2.35 |
| 3 | 2.65 | 2.53 | 2.95 | 2.57 | 2.72 | 2.77 |
| 4 | 2.70 | 2.57 | 2.98 | 2.47 | 2.65 | 2.80 |
| 6 | 2.57 | 2.48 | 2.48 | 2.25 | 2.52 | 2.55 |
| 8 | 2.65 | 2.73 | 2.73 | 2.63 | 2.85 | 3.13 |
| 10 | 3.17 | 3.10 | 2.92 | 2.73 | 2.62 | 3.03 |
| 12 | 3.17 | 3.22 | 3.30 | 2.97 | 3.05 | 3.47 |
| 18 | 3.45 | 3.55 | 3.52 | 3.38 | 3.27 | 3.95 |
| 24 | 2.95 | 3.23 | 2.88 | 3.03 | 3.00 | 4.47 |
| 36 | 4.03 | 3.97 | 3.92 | 4.15 | 3.88 | 4.53 |
| 48 | 4.22 | 4.35 | 3.75 | 3.87 | 3.50 | 4.97 |

As shown in Table 14 and FIG. 4, tablet K (Dorzagliatin, 75 mg) exhibited up to 12 h glucose lowering effects. All tablets A-E (the compound of formula (I), 123 mg) exhibited up to 24 hours of glucose-lowering effects, similar to the BID effects of tablet K, which makes it possible to administer the compound of formula (I) once-a-day.

Besides, IR tablet A, bilayer tablets D and E exhibited fast onset glucose lowering effects similar to tablet K, which is of great importance for diabetes patients.

TABLE 15
Blood Glucose data after oral administration
of different tablets (tablets A-E, K)

| PD parameters | A | B | C | D | E | K |
|---|---|---|---|---|---|---|
| Cₘₐₓ (mmol/L) | 4.53 | 4.68 | 4.50 | 4.53 | 4.73 | 5.08 |
| Cₘᵢₙ (mmol/L) | 2.13 | 2.28 | 2.48 | 2.25 | 2.47 | 2.18 |
| Tₘᵢₙ (h) | 1.0-8.0 | 4.0-6.0 | 6.00 | 6.00 | 6.0-10.0 | 0.5-2.0 |
| AUC₀₋₁₂ | 34.0 | 34.7 | 36.5 | 32.5 | 34.3 | 35.0 |
| (h · mmol/L) | | | | | | |
| AUC₀₋₂₄ | 73.0 | 75.3 | 76.1 | 70.7 | 72.0 | 82.5 |
| (h · mmol/L) | | | | | | |
| *CEₘₐₓ | −2.38 | −1.57 | −1.67 | −1.90 | −1.73 | −2.25 |

*CEₘₐₓ = Cₘᵢₙ − C_{0 h}

As shown in Table 15, tablets A-E have similar mean AUC₀₋₁₂ (h·mmol/L) as tablet K, while tablets A, D and E have lower AUC₀₋₂₄ (h·mmol/L), which also represents better glucose-lowering effects.

The above is a further detailed description of the present disclosure in connection with the specific alternative embodiments, and the specific embodiments of the present disclosure are not limited to the description. For those skilled in the art, without departing from the concept of the present disclosure, some simple deductions or substitutions can be made, which should be regarded as falling within the protection scope of the present disclosure.

1. A pharmaceutical formulation, comprising:
(a) glucokinase activator, or a prodrug, or a pharmaceutically acceptable salt, an isotope labeled analogue, a crystalline form, a hydrate, a solvate, or a diastereomeric or enantiomeric form thereof;
(b) optionally one or more excipients;
wherein the pharmaceutical formulation is an immediate-release formulation;
alternatively, wherein the prodrug is a prodrug of Dorzagliatin, or a pharmaceutically acceptable salt, an isotope labeled analogue, a crystalline form, a hydrate, a solvate, or a diastereomeric or enantiomeric form thereof.

2-75. (canceled)

76. A pharmaceutical formulation comprising at least two parts, wherein:
(i) the first part is an immediate-release pharmaceutical formulation comprising a glucokinase activator, or a prodrug, or a pharmaceutically acceptable salt, an isotope labeled analogue, a crystalline form, a hydrate, a solvate, or a diastereomeric or enantiomeric form thereof, and optionally one or more excipients; and
(ii) wherein the second part is an extended-release formulation comprising the glucokinase activator, or a prodrug, or a pharmaceutically acceptable salt, an isotope labeled analogue, a crystalline form, a hydrate, a solvate, or a diastereomeric or enantiomeric form thereof, and release modifier(s);
or wherein the glucokinase activator, or a prodrug, or a pharmaceutically acceptable salt, an isotope labeled analogue, a crystalline form, a hydrate, a solvate, or a diastereomeric or enantiomeric form thereof is a prodrug of Dorzagliatin, or a pharmaceutically acceptable salt, an isotope labeled analogue, a crystalline form, a hydrate, a solvate, or a diastereomeric or enantiomeric form thereof.

77. The pharmaceutical formulation according to claim 76, wherein the glucokinase activator, or a prodrug, or a pharmaceutically acceptable salt, an isotope labeled analogue, a crystalline form, a hydrate, a solvate, or a diastereomeric or enantiomeric form thereof is a compound of formula (I), 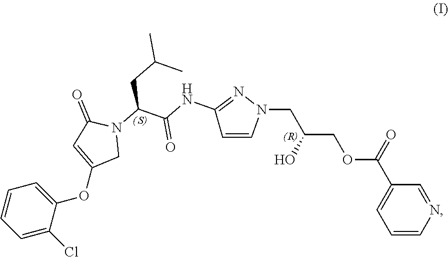
or a pharmaceutically acceptable salt thereof.

78. The pharmaceutical formulation according to claim 77, wherein the release modifier(s) is hydroxypropyl cellulose, hydroxypropyl methylcellulose (HPMC), hydroxyethyl cellulose, hydroxyethyl methyl cellulose, methylcellulose, ethylcellulose, carboxymethylcellulose, polyvinylpyrrolidone, copovidone, polyethylene oxide, polyethylene glycol, carbomer copolymer, carbomer homopolymer, carbomer interpolymer, polymethacrylates, starch, shellac, guar gum, sodium alginate, xanthan gum, alginic acid, or acacia, or a combination thereof.

79. The pharmaceutical formulation according to claim 77, wherein the release modifier(s) is hypromellose E5, hypromellose K100LV, hypromellose K4M, or hypromellose K100M, or a combination thereof.

80. The pharmaceutical formulation according to claim 77, wherein the release modifier(s) is present in a dose of about 10-80%, about 15-45%, about 20-40%, about 25-35%, about 30%, said weight percentage is based on the total weight of the second part.

81. The pharmaceutical formulation according to claim 77, wherein the weight ratio of the compound of formula (I) in the first part to the compound of formula (I) in the second part is about 1:10 to 10:1, about 1:9 to 9:1, about 1:4 to 4:1, about 3:7 to 7:3, about 2:3 to 3:2, about 3:4 to 4:3, about 4:5 to 5:4, or about 5:6 to 6:5; or wherein the ratio is about 2:3 to about 1:1, or about 2:3, or about 1:1.

82. The pharmaceutical formulation according to claim 77, wherein the second part comprises by weight:
about 5-90% of the compound of formula (I), or a pharmaceutically acceptable salt thereof;
about 5-85% of filler(s);
about 10-80% of release modifier(s);
about 0-40% of surfactant(s);
about 0-10% of glidant(s); and
about 0-15% of lubricant(s);
or
about 8-55% of the compound of formula (I), or a pharmaceutically acceptable salt thereof;
about 5-65% of filler(s);
about 15-45% of release modifier(s);
about 1-30% of surfactant(s);
about 0.1-5% of glidant(s); and
about 0.1-10% of lubricant(s);
or
about 40-50% of the compound of formula (I), or a pharmaceutically acceptable salt thereof;
about 5-30% of filler(s);
about 25-35% of release modifier(s);
about 1-25% of surfactant(s);
about 0.1-3% of glidant(s); and
about 0.2-5% of lubricant(s);
or
about 20-25% of the compound of formula (I), or a pharmaceutically acceptable salt thereof;
about 28-35% of filler(s);
about 24-33% of release modifier(s);
about 13-16% of surfactant(s);
about 0.1-3% of glidant(s); and
about 0.2-5% of lubricant(s);
or
about 40-50% of the compound of formula (I), or a pharmaceutically acceptable salt thereof;
about 8-10% of filler(s);
about 24-33% of release modifier(s);
about 13-16% of surfactant(s);
about 0.1-3% of glidant(s); and
about 0.2-5% of lubricant(s);
or
about 32-40% of the compound of formula (I), or a pharmaceutically acceptable salt thereof;
about 7-10% of filler(s);
about 26-33% of release modifier(s);
about 21-26% of surfactant(s);
about 0.1-3% of glidant(s); and
about 0.2-5% of lubricant(s).

83. The pharmaceutical formulation according to claim 77, wherein
(i) the first part comprises by weight:
about 5-60% of the compound of formula (I), or a pharmaceutically acceptable salt thereof;
about 30-85% of filler(s);
about 0.5-25% of disintegrant(s);
about 0-15% of surfactant(s);
about 0-10% of glidant(s); and
about 0-15% of lubricant(s);
said percentage based on the total weight of the first part; and
(ii) the second part comprises by weight:
about 8-55% of the compound of formula (I), or a pharmaceutically acceptable salt thereof;
about 5-65% of filler(s);
about 15-45% of release modifier(s);
about 1-30% of surfactant(s);
about 0.1-3% of glidant(s); and
about 0.1-10% of lubricant(s);
said percentage based on the total weight of the second part.

84. The pharmaceutical formulation according to claim 77, wherein the pharmaceutical formulation is a tablet, a granule, a pill, or a capsule; or wherein the pharmaceutical formulation is a bilayer tablet.

85. The pharmaceutical formulation according to claim 77, wherein the pharmaceutical formulation is a bilayer tablet, and wherein:
(i) the first part is an immediate-release layer comprising by weight:
about 5-60% of the compound of formula (I), or a pharmaceutically acceptable salt thereof;
about 30-85% of silicified microcrystalline cellulose;
about 0-25% of croscarmellose sodium;
about 0-15% of sodium lauryl sulfate;
about 0-10% of colloidal silicone dioxide; and
about 0-15% of magnesium stearate;
said percentage based on the total weight of the immediate-release layer; and
(ii) the second part is an extended-release layer comprising by weight:
about 8-55% of the compound of formula (I), or a pharmaceutically acceptable salt thereof;
about 5-65% of silicified microcrystalline cellulose;
about 15-45% of hypromellose E5;
about 1-30% of sodium lauryl sulfate;
about 0.1-3% of colloidal silicone dioxide; and
about 0.1-10% of magnesium stearate;
or
about 8-55% of the compound of formula (I), or a pharmaceutically acceptable salt thereof;
about 5-65% of silicified microcrystalline cellulose;
about 10-20% of hypromellose E5;
about 10-20% hypromellose K100LV;
about 1-30% of sodium lauryl sulfate;
about 0.1-3% of colloidal silicone dioxide; and
about 0.1-10% of magnesium stearate;
or
about 8-55% of the compound of formula (I), or a pharmaceutically acceptable salt thereof;
about 5-65% of silicified microcrystalline cellulose;
about 15-45% of hypromellose K100LV;
about 1-30% of sodium lauryl sulfate;
about 0.1-3% of colloidal silicone dioxide; and
about 0.1-10% of magnesium stearate;
or
about 8-55% of the compound of formula (I), or a pharmaceutically acceptable salt thereof;
about 5-65% of silicified microcrystalline cellulose;
about 15-45% of hypromellose K100M;
about 0-30% of sodium lauryl sulfate;
about 0.1-3% of colloidal silicone dioxide; and
about 0.1-10% of magnesium stearate;
said percentage based on the total weight of the extended-release layer;
or
about 8-55% of the compound of formula (I), or a pharmaceutically acceptable salt thereof;
about 5-65% of silicified microcrystalline cellulose;
about 15-45% of hypromellose K4M;
about 1-30% of sodium lauryl sulfate;
about 0.1-3% of colloidal silicone dioxide; and
about 0.1-10% of magnesium stearate;
said percentage based on the total weight of the extended-release layer.

86. The pharmaceutical formulation according to claim 77, wherein the pharmaceutical formulation is a bilayer tablet, and wherein:
(i) the first part is an immediate-release layer, comprising the components by weight:
(a)
about 40-50% of the compound of formula (I), or a corresponding amount of the pharmaceutically acceptable salt thereof,
about 42-52% of silicified microcrystalline cellulose;
about 0.5-10% of croscarmellose sodium;
about 1-8% of sodium lauryl sulfate;
about 0.1-3% of colloidal silicone dioxide; and
about 0.2-5% of magnesium stearate;
(b)
about 30-37% of the compound of formula (I), or a corresponding amount of the pharmaceutically acceptable salt thereof,
about 55-70% of silicified microcrystalline cellulose;
about 0.5-10% of croscarmellose sodium;
about 0-8% of sodium lauryl sulfate;
about 0-3% of colloidal silicone dioxide; and
about 0-5% of magnesium stearate;
(c)
about 20-25% of the compound of formula (I), or a corresponding amount of the pharmaceutically acceptable salt thereof,
about 60-76% of silicified microcrystalline cellulose;
about 0.5-10% of croscarmellose sodium;
about 1-8% of sodium lauryl sulfate;
about 0.1-3% of colloidal silicone dioxide; and
about 0.2-5% of magnesium stearate;
or (d)
about 10-12% of the compound of formula (I), or a corresponding amount of the pharmaceutically acceptable salt thereof,
about 70-85% of silicified microcrystalline cellulose;
about 0.5-10% of croscarmellose sodium;
about 1-8% of sodium lauryl sulfate;
about 0.1-3% of colloidal silicone dioxide; and
about 0.2-5% of magnesium stearate;
said percentage based on the total weight of the immediate-release layer;
and (ii) the second part is an extended-release layer comprising by weight:
(A) about 40-50% of the compound of formula (I), or a corresponding amount of the pharmaceutically acceptable salt thereof, about 8.5-10% of silicified microcrystalline cellulose, about 25-35% of hypromellose K100M, about 13-16% of sodium lauryl sulfate, about 0.1-3% of colloidal silicone dioxide, and about 0.2-5% of magnesium stearate, said percentage based on the total weight of the second layer;
(B) about 40-50% of the compound of formula (I), or a corresponding amount of the pharmaceutically acceptable salt thereof, about 18-22% of silicified microcrystalline cellulose, about 25-35% of hypromellose K100M, about 3-4% of sodium lauryl sulfate, about 0.1-3% of colloidal silicone dioxide, and about 0.2-5% of magnesium stearate, said percentage based on the total weight of the extended-release layer;
(C) about 40-50% of the compound of formula (I), or a corresponding amount of the pharmaceutically acceptable salt thereof, about 8.5-10% of silicified microcrystalline cellulose, about 25-35% of hypromellose E5, about 13-16% of sodium lauryl sulfate, about 0.1-3% of colloidal silicone dioxide, and about 0.2-5% of magnesium stearate, said percentage based on the total weight of the extended-release layer;
(D) about 40-50% of the compound of formula (I), or a corresponding amount of the pharmaceutically acceptable salt thereof, about 8.5-10% of silicified microcrystalline cellulose, about 13-17% of hypromellose E5, about 13-17% of hypromellose K100LV, about 13-16% of sodium lauryl sulfate, about 0.1-3% of colloidal silicone dioxide, and about 0.2-5% of magnesium stearate, said percentage based on the total weight of the extended-release layer;
(E) about 40-50% of the compound of formula (I), or a corresponding amount of the pharmaceutically acceptable salt thereof, about 8.5-10% of silicified microcrystalline cellulose, about 25-35% of hypromellose100LV, about 13-16% of sodium lauryl sulfate, about 0.1-3% of colloidal silicone dioxide, and about 0.2-5% of magnesium stearate, said percentage based on the total weight of the extended-release layer; or
(F) about 40-50% of the compound of formula (I), or a corresponding amount of the pharmaceutically acceptable salt thereof, about 21-27% of silicified microcrystalline cellulose, about 25-35% of hypromellose K100M, about 0.1-3% of colloidal silicone dioxide, and about 0.2-5% of magnesium stearate;
said percentage based on the total weight of the extended-release layer.

87. The pharmaceutical formulation of claim 86, wherein the immediate-release layer comprises:
(a) about 44.73% of the compound of formula (I), or a corresponding amount of the pharmaceutically acceptable salt thereof; about 47.09% of silicified microcrystalline cellulose; about 3.09% of croscarmellose sodium; about 3.64% of sodium lauryl sulfate; about 0.54% of colloidal silicone dioxide; and about 0.91% of magnesium stearate, said percentage based on the total weight of the immediate-release layer;
(b) about 33.5% of the compound of formula (I), or a corresponding amount of the pharmaceutically acceptable salt thereof, about 62.4% of silicified microcrystalline cellulose; about 3.1% of croscarmellose sodium; and about 1.0% of magnesium stearate, said percentage based on the total weight of the immediate-release layer;
(c) about 22.4% of the compound of formula (I), or a corresponding amount of the pharmaceutically acceptable salt thereof, about 69.5% of silicified microcrystalline cellulose; about 3.1% of croscarmellose sodium; about 3.6% of sodium lauryl sulfate; about 0.5% of colloidal silicone dioxide; and about 0.9% of magnesium stearate, said percentage based on the total weight of the immediate-release layer; or
(d) about 11.2% of the compound of formula (I), or a corresponding amount of the pharmaceutically acceptable salt thereof, about 80.6% of silicified microcrystalline cellulose; about 3.1% of croscarmellose sodium; about 3.6% of sodium lauryl sulfate; about 0.5% of colloidal silicone dioxide; and about 0.9% of magnesium stearate, said percentage based on the total weight of the immediate-release layer;
the extended-release layer comprising:
(A) about 44.73% of the compound of formula (I), or a corresponding amount of the pharmaceutically acceptable salt thereof, about 9.45% of silicified microcrystalline cellulose;
about 29.82% of hypromellose K100M; about 14.55% of sodium lauryl sulfate; about 0.54% of colloidal silicone dioxide; and about 0.91% of magnesium stearate, said percentage based on the total weight of the extended-release layer; or
(B) about 44.73% of the compound of formula (I), or a corresponding amount of the pharmaceutically acceptable salt thereof; about 20.36% of silicified microcrystalline cellulose; about 29.82% of hypromellose K100M; about 3.64% of sodium lauryl sulfate; about 0.54% of colloidal silicone dioxide; and about 0.91% of magnesium stearate, said percentage based on the total weight of the extended-release layer;
(C) about 44.7% of the compound of formula (I), or a corresponding amount of the pharmaceutically acceptable salt thereof, about 9.5% of silicified microcrystalline cellulose; about 29.8% of hypromellose E5; about 14.5% of sodium lauryl sulfate; about 0.5% of colloidal silicone dioxide; and about 0.9% of magnesium stearate, said percentage based on the total weight of the extended-release layer;
(D) about 44.7% of the compound of formula (I), or a corresponding amount of the pharmaceutically acceptable salt thereof, about 9.5% of silicified microcrystalline cellulose; about 14.9% of hypromellose E5; about 14.9% of hypromellose K100LV; about 14.5% of sodium lauryl sulfate; about 0.5% of colloidal silicone dioxide; and about 0.9% of magnesium stearate, said percentage based on the total weight of the extended-release layer;
(E) about 44.7% of the compound of formula (I), or a corresponding amount of the pharmaceutically acceptable salt thereof, about 9.5% of silicified microcrystalline cellulose; about 29.8% of hypromellose K100LV; about 14.5% of sodium lauryl sulfate; about 0.5% of colloidal silicone dioxide; and about 0.9% of magnesium stearate, said percentage based on the total weight of the extended-release layer;
or (F) about 44.7% of the compound of formula (I), or a corresponding amount of the pharmaceutically acceptable salt thereof; about 24% of silicified microcrystalline cellulose; about 29.8% of hypromellose K100M; about 1.5% of colloidal silicone dioxide; and about 2.5% of magnesium stearate, said percentage based on the total weight of the extended-release layer.

88. The pharmaceutical formulation of claim 77, wherein the formulation is a bilayer tablet, and wherein:
the first part is an immediate-release layer comprising by weight:
(h)
about 16-20% of the compound of formula (I), or a corresponding amount of the pharmaceutically acceptable salt thereof,
about 66-81% of silicified microcrystalline cellulose;
about 2.5-3.5% of croscarmellose sodium;
about 3-4% of sodium lauryl sulfate;
about 0.1-3% of colloidal silicone dioxide; and
about 0.2-5% of magnesium stearate;
(i)
about 32-40% of the compound of formula (I), or a corresponding amount of the pharmaceutically acceptable salt thereof,
about 49-60% of silicified microcrystalline cellulose;
about 2.5-3.5% of croscarmellose sodium;
about 3-4% of sodium lauryl sulfate;
about 0.1-3% of colloidal silicone dioxide; and
about 0.2-5% of magnesium stearate;
(j)
about 32-40% of the compound of formula (I), or a corresponding amount of the pharmaceutically acceptable salt thereof,
about 46-57% of silicified microcrystalline cellulose;
about 2.5-3.5% of croscarmellose sodium;
about 6-8% of sodium lauryl sulfate;
about 0.1-3% of colloidal silicone dioxide; and
about 0.2-5% of magnesium stearate;
or (i-f)
about 18-27% of the compound of formula (I), or a corresponding amount of the pharmaceutically acceptable salt thereof,
about 56-83% of silicified microcrystalline cellulose;
about 0.5-10% of croscarmellose sodium;
about 1-8% of sodium lauryl sulfate;
about 0.1-3% of colloidal silicone dioxide; and
about 0.2-5% of magnesium stearate;
said percentage based on the total weight of the immediate-release layer;
and the second part is an extended-release layer comprising by weight:
(E-10) about 40-50% of the compound of formula (I), or a corresponding amount of the pharmaceutically acceptable salt thereof, about 8-10% of silicified microcrystalline cellulose, about 24-33% of hypromellose K100M, about 13-16% of sodium lauryl sulfate, about 0.1-3% of colloidal silicone dioxide, and about 0.2-5% of magnesium stearate;
(E-9) about 20-25% of the compound of formula (I), or a corresponding amount of the pharmaceutically acceptable salt thereof, about 28-35% of silicified microcrystalline cellulose, about 24-33% of hypromellose K100M, about 13-16% of sodium lauryl sulfate, about 0.1-3% of colloidal silicone dioxide, and about 0.2-5% of magnesium stearate;
(E-11) about 32-40% of the compound of formula (I), or a corresponding amount of the pharmaceutically acceptable salt thereof, about 7-10% of silicified microcrystalline cellulose, about 26-33% of hypromellose K100M, about 21-26% of sodium lauryl sulfate, about 0.1-3% of colloidal silicone dioxide, and about 0.2-5% of magnesium stearate, said percentage based on the total weight of the extended-release layer;
(E-12) about 32-40% of the compound of formula (I), or a corresponding amount of the pharmaceutically acceptable salt thereof, about 7-10% of silicified microcrystalline cellulose, about 26-33% of hypromellose K4M, about 21-26% of sodium lauryl sulfate, about 0.1-3% of colloidal silicone dioxide, and about 0.2-5% of magnesium stearate, said percentage based on the total weight of the extended-release layer; said percentage based on the total weight of the extended-release layer;
(E-13) about 32-40% of the compound of formula (I), or a corresponding amount of the pharmaceutically acceptable salt thereof, about 7-10% of silicified microcrystalline cellulose, about 26-33% of hypromellose K100LV, about 21-26% of sodium lauryl sulfate, about 0.1-3% of colloidal silicone dioxide, and about 0.2-5% of magnesium stearate, said percentage based on the total weight of the extended-release layer; said percentage based on the total weight of the extended-release layer; or
(E-f) about 18-27% of the compound of formula (I), or a corresponding amount of the pharmaceutically acceptable salt thereof, about 25-38% of silicified microcrystalline cellulose, about 25-35% of hypromellose K100M, about 13-16% of sodium lauryl sulfate, about 0.1-3% of colloidal silicone dioxide; and about 0.2-5% of magnesium stearate, said percentage based on the total weight of the second layer.

89. The pharmaceutical formulation according to claim 88, wherein the immediate-release layer comprises:
(h)
about 18.22% of the compound of formula (I), or a corresponding amount of the pharmaceutically acceptable salt thereof,
about 73.11% of silicified microcrystalline cellulose;
about 3.09% of croscarmellose sodium;
about 3.64% of sodium lauryl sulfate;
about 0.55% of colloidal silicone dioxide; and
about 1.39% of magnesium stearate;
(i)
about 36.44% of the compound of formula (I), or a corresponding amount of the pharmaceutically acceptable salt thereof,
about 54.47% of silicified microcrystalline cellulose;
about 3.09% of croscarmellose sodium;
about 3.64% of sodium lauryl sulfate;
about 0.55% of colloidal silicone dioxide; and
about 1.81% of magnesium stearate;
(j)
about 36.18% of the compound of formula (I), or a corresponding amount of the pharmaceutically acceptable salt thereof,
about 51.38% of silicified microcrystalline cellulose;
about 3.09% of croscarmellose sodium;
about 7.06% of sodium lauryl sulfate;
about 0.53% of colloidal silicone dioxide; and
about 1.76% of magnesium stearate;
or (i-f)
about 22.36% of the compound of formula (I), or a corresponding amount of the pharmaceutically acceptable salt thereof,
about 69.45% of silicified microcrystalline cellulose;
about 3.09% of croscarmellose sodium;
about 3.64% of sodium lauryl sulfate;
about 0.54% of colloidal silicone dioxide; and
about 0.91% of magnesium stearate;
said percentage based on the total weight of the immediate-release layer;
and wherein the extended-release layer comprises:
(E-10) about 44.73% of the compound of formula (I), or a corresponding amount of the pharmaceutically acceptable salt thereof, about 8.86% of silicified microcrystalline cellulose;
about 29.82% of hypromellose K100M; about 14.55% of sodium lauryl sulfate; about 0.55% of colloidal silicone dioxide; and about 1.50% of magnesium stearate;
(E-9) about 22.36% of the compound of formula (I), or a corresponding amount of the pharmaceutically acceptable salt thereof; about 31.52% of silicified microcrystalline cellulose;
about 29.82% of hypromellose K100M; about 14.55% of sodium lauryl sulfate; about 0.55% of colloidal silicone dioxide; and about 1.21% of magnesium stearate;
(E-11) about 36.18% of the compound of formula (I), or a corresponding amount of the pharmaceutically acceptable salt thereof, about 8.71% of silicified microcrystalline cellulose; about 29.59% of hypromellose K100M; about 23.53% of sodium lauryl sulfate; about 0.53% of colloidal silicone dioxide; and about 1.47% of magnesium stearate;
(E-12) about 36.18% of the compound of formula (I), or a corresponding amount of the pharmaceutically acceptable salt thereof, about 8.71% of silicified microcrystalline cellulose; about 29.59% of hypromellose K4M; about 23.53% of sodium lauryl sulfate; about 0.53% of colloidal silicone dioxide; and about 1.47% of magnesium stearate;
(E-13) about 36.18% of the compound of formula (I), or a corresponding amount of the pharmaceutically acceptable salt thereof, about 8.71% of silicified microcrystalline cellulose; about 29.59% of hypromellose K100LV; about 23.53% of sodium lauryl sulfate; about 0.53% of colloidal silicone dioxide; and about 1.47% of magnesium stearate; or
(E-f) about 22.36% of the compound of formula (I), or a corresponding amount of the pharmaceutically acceptable salt thereof; about 31.82% of silicified microcrystalline cellulose; about 29.82% of hypromellose K100M; about 14.55% of sodium lauryl sulfate; about 0.54% of colloidal silicone dioxide; and about 0.91% of magnesium stearate;
said percentage based on the total weight of the extended-release layer.

90. The pharmaceutical formulation of claim 77, wherein the weight ratio of the first part to the second part is about 1:10 to 10:1, about 1:9 to 9:1, about 1:4 to 4:1, about 3:7 to 7:3, about 2:3 to 3:2, about 3:4 to 4:3, about 4:5 to 5:4 or about 5:6 to 6:5; or wherein the weight ratio is about 1:1, about 1:2, about 2:3, about 3:4, about 4:5, about 5:6, about 2:1, about 3:2, about 4:3, about 5:4 or about 6:5.

91. The pharmaceutical formulation of claim 77, wherein the pharmaceutical formulation is a bilayer tablet, wherein he first part is a first layer comprising:
about 16-20% of the compound of formula (I), or a corresponding amount of the pharmaceutically acceptable salt thereof,
about 66-81% of filler(s);
about 2.5-3.5% of disintegrant(s);
about 3-4% of surfactant(s);
about 0.1-3% of glidant(s); and
about 0.2-5% of lubricant(s);
said percentage based on the total weight of the first layer;
wherein the second part is a second layer comprising:
about 20-25% of the compound of formula (I), or a pharmaceutically acceptable salt thereof;
about 28-35% of filler(s);
about 24-33% of release modifier(s);
about 13-16% of surfactant(s);
about 0.1-3% of glidant(s); and
about 0.2-5% of lubricant(s);
said percentage based on the total weight of the second layer;
and wherein the weight ratio of the compound of formula (I) in the first part to the compound of formula (I) in the second part is from about 1:4 to about 3:2;
optionally the total weight of the compound of formula (I) in the pharmaceutical formulation is no more than about 92.25 mg.

92. The pharmaceutical formulation according to claim 91, wherein the pharmaceutical formulation is a bilayer tablet, wherein the first part is a first layer comprising:
about 18.22% of the compound of formula (I), or a corresponding amount of the pharmaceutically acceptable salt thereof,
about 73.11% of filler(s);
about 3.09% of disintegrant(s);
about 3.64% of surfactant(s);
about 0.55% of glidant(s); and
about 1.39% of lubricant(s);
said percentage based on the total weight of the first layer;
wherein the second part is a second layer comprising:
about 22.36% of the compound of formula (I), or a corresponding amount of the pharmaceutically acceptable salt thereof,
about 31.52% of filler(s);
about 29.82% of release modifier(s);
about 14.55% of surfactant(s);
about 0.55% of glidant(s); and
about 1.21% of lubricant(s);
said percentage based on the total weight of the second layer;
and wherein the weight ratio of the compound of formula (I) in the first part to the compound of formula (I) in the second part is from about 1:4 to about 3:2;
optionally the total weight of the compound of formula (I) in the pharmaceutical formulation is no more than about 92.25 mg.

93. The pharmaceutical formulation of claim 77, wherein the pharmaceutical formulation is a bilayer tablet, wherein the first part is a first layer comprising:
about 32-40% of the compound of formula (I), or a corresponding amount of the pharmaceutically acceptable salt thereof,
about 49-60% of filler(s);
about 2.5-3.5% of disintegrant(s);
about 34% of surfactant(s);
about 0.1-3% of glidant(s); and
about 0.2-5% of lubricant(s);
said percentage based on the total weight of the first layer;
wherein the second part is a second layer comprising:
about 40-50% of the compound of formula (I), or a pharmaceutically acceptable salt thereof;
about 8-10% of filler(s);
about 24-33% of release modifier(s);
about 13-16% of surfactant(s);
about 0.1-3% of glidant(s); and
about 0.2-5% of lubricant(s);
said percentage based on the total weight of the second layer;
and wherein the weight ratio of the compound of formula (I) in the first part to the compound of formula (I) in the second part is from about 1:4 to about 3:2;
optionally the total weight of the compound of formula (I) in the pharmaceutical formulation is from about 92.25 mg to about 153.75 mg.

94. The pharmaceutical formulation according to claim 93, wherein the pharmaceutical formulation is a bilayer tablet, wherein the first part is a first layer comprising:
about 36.44% of the compound of formula (I), or a corresponding amount of the pharmaceutically acceptable salt thereof,
about 54.47% of filler(s);
about 3.09% of disintegrant(s);
about 3.64% of surfactant(s);
about 0.55% of glidant(s); and
about 1.81% of lubricant(s);
said percentage based on the total weight of the first layer;
wherein the second part is a second layer comprising:
about 44.73% of the compound of formula (I), or a corresponding amount of the pharmaceutically acceptable salt thereof,
about 8.86% of filler(s);
about 29.82% of release modifier(s);
about 14.55% of surfactant(s);
about 0.55% of glidant(s); and
about 1.50% of lubricant(s);
said percentage based on the total weight of the second layer;
and wherein the weight ratio of the compound of formula (I) in the first part to the compound of formula (I) in the second part is from about 1:4 to about 3:2;
optionally the total weight of the compound of formula (I) in the pharmaceutical formulation is from about 92.25 mg to about 153.75 mg.

95. The pharmaceutical formulation of claim 77, wherein the pharmaceutical formulation is a bilayer tablet, wherein the first part is a first layer comprising:
about 32-40% of the compound of formula (I), or a corresponding amount of the pharmaceutically acceptable salt thereof,
about 46-57% of filler(s);
about 2.5-3.5% of disintegrant(s);
about 6-8% of surfactant(s);
about 0.1-3% of colloidal silicone dioxide; and
about 0.2-5% of lubricant(s);
said percentage based on the total weight of the first layer;
wherein the second part is a second layer comprising:
about 32-40% of the compound of formula (I), or a pharmaceutically acceptable salt thereof;
about 7-10% of filler(s);
about 26-33% of release modifier(s);
about 21-26% of surfactant(s);
about 0.1-3% of glidant(s); and
about 0.2-5% of lubricant(s);
said percentage based on the total weight of the second layer;
and wherein the weight ratio of the compound of formula (I) in the first part to the compound of formula (I) in the second part is from about 1:4 to about 3:2;
optionally the total weight of the compound of formula (I) in the pharmaceutical formulation is from about 92.25 mg to about 153.75 mg, from about 153.75 mg to about 215.25 mg, or no less than about 215.25 mg, or is from about 215.25 mg to about 276.75 mg.

96. The pharmaceutical formulation according to claim 95, wherein the pharmaceutical formulation is a bilayer tablet, wherein the first part is a first layer comprising:
about 36.18% of the compound of formula (I), or a corresponding amount of the pharmaceutically acceptable salt thereof,
about 51.38% of filler(s);
about 3.09% of disintegrant(s);
about 7.06% of surfactant(s);
about 0.53% of glidant(s); and
about 1.76% of lubricant(s);
said percentage based on the total weight of the first layer;
wherein the second part is a second layer comprising:
about 36.18% of the compound of formula (I), or a corresponding amount of the pharmaceutically acceptable salt thereof,
about 8.71% of filler(s);
about 29.59% of release modifier(s);
about 23.53% of surfactant(s);
about 0.53% of glidant(s); and
about 1.47% of lubricant(s);
said percentage based on the total weight of the second layer;
and wherein the weight ratio of the compound of formula (I) in the first part to the compound of formula (I) in the second part is from about 1:4 to about 3:2;
optionally the total weight of the compound of formula (I) in the pharmaceutical formulation is from about 92.25 mg to about 153.75 mg, from about 153.75 mg to about 215.25 mg, or no less than about 215.25 mg, or is from about 215.25 mg to about 276.75 mg.

97. The pharmaceutical formulation of claim 77, wherein the first layer comprises:
about 10-100 mg of the compound of formula (I), or a corresponding amount of the pharmaceutically acceptable salt thereof;
about 30-150 mg of silicified microcrystalline cellulose;
about 0-10 mg of croscarmellose sodium;
about 0-10 mg of sodium lauryl sulfate;
about 0-5 mg of colloidal silicone dioxide; and
about 0.1-10 mg of magnesium stearate;
or wherein the first layer comprises:
(a)
about 40-61.5 mg of the compound of formula (I), or a corresponding amount of the pharmaceutically acceptable salt thereof;
about 40-65 mg of silicified microcrystalline cellulose;
about 2-5 mg of croscarmellose sodium;
about 2-15 mg of sodium lauryl sulfate;
about 0.1-3 mg of colloidal silicone dioxide; and
about 0.1-5 mg of magnesium stearate;
(b)
about 30-45 mg of the compound of formula (I), or a corresponding amount of the pharmaceutically acceptable salt thereof;
about 50-85 mg of silicified microcrystalline cellulose;
about 2-5 mg of croscarmellose sodium; and
about 0.1-3 mg of magnesium stearate;
(c)
about 20-30 mg of the compound of formula (I), or a corresponding amount of the pharmaceutically acceptable salt thereof;
about 60-95 mg of silicified microcrystalline cellulose;
about 2-5 mg of croscarmellose sodium;
about 2-15 mg of sodium lauryl sulfate;
about 0.1-3 mg of colloidal silicone dioxide; and
about 0.1-5 mg of magnesium stearate;
or (d)
about 10-15 mg of the compound of formula (I), or a corresponding amount of the pharmaceutically acceptable salt thereof;
about 70-110 mg of silicified microcrystalline cellulose;
about 2-5 mg of croscarmellose sodium;
about 2-15 mg of sodium lauryl sulfate;
about 0.1-3 mg of colloidal silicone dioxide; and
about 0.1-5 mg of magnesium stearate;
and wherein the second layer comprises:
(A)
about 60-90 mg of the compound of formula (I), or a corresponding amount of the pharmaceutically acceptable salt thereof;
about 10-30 mg of silicified microcrystalline cellulose;
about 35-60 mg of hypromellose K100M;
about 15-30 mg of sodium lauryl sulfate;
about 0.1-3 mg of colloidal silicone dioxide; and
about 0.1-5 mg of magnesium stearate;
(B)
about 60-90 mg of the compound of formula (I), or a corresponding amount of the pharmaceutically acceptable salt thereof;
about 20-40 mg of silicified microcrystalline cellulose;
about 35-60 mg of hypromellose K100M;
about 3-15 mg of sodium lauryl sulfate;
about 0.1-3 mg of colloidal silicone dioxide; and
about 0.1-5 mg of magnesium stearate;
(C)
about 60-90 mg of the compound of formula (I), or a corresponding amount of the pharmaceutically acceptable salt thereof;
about 10-30 mg of silicified microcrystalline cellulose;
about 35-60 mg of hypromellose E5;
about 15-30 mg of sodium lauryl sulfate;
about 0.1-3 mg of colloidal silicone dioxide; and
about 0.1-5 mg of magnesium stearate;
(D)
about 60-90 mg of the compound of formula (I), or a corresponding amount of the pharmaceutically acceptable salt thereof;
about 10-30 mg of silicified microcrystalline cellulose;
about 15-30 mg of hypromellose E5;
about 15-30 mg of hypromellose K100LV;
about 15-30 mg of sodium lauryl sulfate;
about 0.1-3 mg of colloidal silicone dioxide; and
about 0.1-5 mg of magnesium stearate;
(E)
about 60-90 mg of the compound of formula (I), or a corresponding amount of the pharmaceutically acceptable salt thereof;
about 10-30 mg of silicified microcrystalline cellulose;
about 35-60 mg of hypromellose K100LV
about 15-30 mg of sodium lauryl sulfate;
about 0.1-3 mg of colloidal silicone dioxide; and
about 0.1-5 mg of magnesium stearate;
or (F)
about 60-90 mg of the compound of formula (I), or a corresponding amount of the pharmaceutically acceptable salt thereof;
about 30-50 mg of silicified microcrystalline cellulose;
about 35-60 mg of hypromellose K100M
about 0.1-3 mg of colloidal silicone dioxide; and
about 0.1-5 mg of magnesium stearate.

98. The pharmaceutical formulation of claim 77, wherein the first layer comprises:
(a)
about 49.2 mg of the compound of formula (I), or a corresponding amount of the pharmaceutically acceptable salt thereof;
about 51.8 mg of silicified microcrystalline cellulose;
about 3.4 mg of croscarmellose sodium;
about 4.0 mg of sodium lauryl sulfate;
about 0.6 mg of colloidal silicone dioxide; and
about 1.0 mg of magnesium stearate;
(b)
about 36.8 mg of the compound of formula (I), or a corresponding amount of the pharmaceutically acceptable salt thereof;
about 68.68 mg of silicified microcrystalline cellulose;
about 3.4 mg of croscarmellose sodium; and
about 1.12 mg of magnesium stearate;
(c)
about 24.6 mg of the compound of formula (I), or a corresponding amount of the pharmaceutically acceptable salt thereof;
about 76.4 mg of silicified microcrystalline cellulose;
about 3.4 mg of croscarmellose sodium;
about 4.0 mg of sodium lauryl sulfate;
about 0.6 mg of colloidal silicone dioxide; and
about 1.0 mg of magnesium stearate;
or (d)
about 12.3 mg of the compound of formula (I), or a corresponding amount of the pharmaceutically acceptable salt thereof;
about 88.7 mg of silicified microcrystalline cellulose;
about 3.4 mg of croscarmellose sodium;
about 4.0 mg of sodium lauryl sulfate;
about 0.6 mg of colloidal silicone dioxide; and
about 1.0 mg of magnesium stearate;
and wherein the second layer comprises:
(A)
about 73.8 mg of the compound of formula (I), or a corresponding amount of the pharmaceutically acceptable salt thereof;
about 15.6 mg of silicified microcrystalline cellulose;
about 49.2 mg of hypromellose K100M;
about 24 mg of sodium lauryl sulfate;
about 0.9 mg of colloidal silicone dioxide; and
about 1.5 mg of magnesium stearate;
(B)
about 73.8 mg of the compound of formula (I), or a corresponding amount of the pharmaceutically acceptable salt thereof;
about 33.6 mg of silicified microcrystalline cellulose;
about 49.2 mg of hypromellose K100M;
about 6.0 mg of sodium lauryl sulfate;
about 0.9 mg of colloidal silicone dioxide; and
about 1.5 mg of magnesium stearate;
(C)
about 73.8 mg of the compound of formula (I), or a corresponding amount of the pharmaceutically acceptable salt thereof;
about 15.6 mg of silicified microcrystalline cellulose;
about 49.2 mg of hypromellose E5;
about 24 mg of sodium lauryl sulfate;
about 0.9 mg of colloidal silicone dioxide; and
about 1.5 mg of magnesium stearate;
(D)
about 73.8 mg of the compound of formula (I), or a corresponding amount of the pharmaceutically acceptable salt thereof;
about 15.6 mg of silicified microcrystalline cellulose;
about 24.6 mg of hypromellose E5;
about 24.6 mg of hypromellose K100LV;
about 24 mg of sodium lauryl sulfate;
about 0.9 mg of colloidal silicone dioxide; and
about 1.5 mg of magnesium stearate;
(E)
about 73.8 mg of the compound of formula (I), or a corresponding amount of the pharmaceutically acceptable salt thereof;
about 15.6 mg of silicified microcrystalline cellulose;
about 49.2 mg of hypromellose K100LV;
about 24 mg of sodium lauryl sulfate;
about 0.9 mg of colloidal silicone dioxide; and
about 1.5 mg of magnesium stearate;
or (F)
about 73.8 mg of the compound of formula (I) or a corresponding amount of the pharmaceutically acceptable salt thereof,
about 39.6 mg of silicified microcrystalline cellulose;
about 49.2 mg of hypromellose K100M
about 0.9 mg of colloidal silicone dioxide; and
about 1.5 mg of magnesium stearate.

99. The pharmaceutical formulation of claim 77, wherein the first layer comprises:
about 15-265 mg of the compound of formula (I), or a corresponding amount of the pharmaceutically acceptable salt thereof;
about 30-250 mg of silicified microcrystalline cellulose;
about 0-15 mg of croscarmellose sodium;
about 0-35 mg of sodium lauryl sulfate;
about 0-5 mg of colloidal silicone dioxide; and
about 0.1-10 mg of magnesium stearate;
or:
about 15-150 mg of the compound of formula (I), or a corresponding amount of the pharmaceutically acceptable salt thereof;
about 50-210 mg of silicified microcrystalline cellulose;
about 1-15 mg of croscarmellose sodium;
about 1-30 mg of sodium lauryl sulfate;
about 0-5 mg of colloidal silicone dioxide; and
about 0-10 mg of magnesium stearate;
(e)
about 19-30 mg the compound of formula (I), or a corresponding amount of the pharmaceutically acceptable salt thereof,
about 78-120 mg silicified microcrystalline cellulose;
about 3-6 mg croscarmellose sodium;
about 3-6 mg sodium lauryl sulfate;
about 0.1-3 mg colloidal silicone dioxide; and
about 1.5-6 mg of magnesium stearate;
(f)
about 39-60 mg the compound of formula (I), or a corresponding amount of the pharmaceutically acceptable salt thereof,
about 56-89 mg silicified microcrystalline cellulose;
about 2-5 mg croscarmellose sodium;
about 4-12 mg sodium lauryl sulfate;
about 0.1-3 mg colloidal silicone dioxide; and
about 1.5-6 mg of magnesium stearate;
(g)
about 58-120 mg the compound of formula (I), or a corresponding amount of the pharmaceutically acceptable salt thereof;
about 83-168 mg silicified microcrystalline cellulose;
about 5-10 mg croscarmellose sodium;
about 11-23 mg sodium lauryl sulfate;
about 0.1-3 mg colloidal silicone dioxide; and
about 1.5-6 mg of magnesium stearate;
or (h)
about 98-148 mg of the compound of formula (I), or a corresponding amount of the pharmaceutically acceptable salt thereof;
about 139-210 mg of silicified microcrystalline cellulose;
about 8-13 mg of croscarmellose sodium;
about 19-30 mg of sodium lauryl sulfate;
about 0.1-3 mg of colloidal silicone dioxide; and
about 4-8 mg of magnesium stearate;
and wherein the second layer comprises:
(G) about 29-45 mg the compound of formula (I), or a corresponding amount of the pharmaceutically acceptable salt thereof;
about 41-63 mg silicified microcrystalline cellulose;
about 39-60 mg hypromellose K100M;
about 19-30 mg of sodium lauryl sulfate;
about 0.1-3 mg of colloidal silicone dioxide; and
about 1-5 mg of magnesium stearate;
(H)
about 60-90 mg of the compound of formula (I), or a corresponding amount of the pharmaceutically acceptable salt thereof;
about 11-18 mg silicified microcrystalline cellulose;
about 39-60 mg hypromellose K100M;
about 19-30 mg sodium lauryl sulfate;
about 0.1-3 mg colloidal silicone dioxide; and
about 1-5 mg magnesium stearate;
(I)
about 60-90 mg of the compound of formula (I), or a corresponding amount of the pharmaceutically acceptable salt thereof;
about 14-22 mg silicified microcrystalline cellulose;
about 48-73 mg hypromellose K100M;
about 38-58 mg sodium lauryl sulfate;
about 0.1-3 mg colloidal silicone dioxide; and
about 1-5 mg magnesium stearate;
(J)
about 60-90 mg of the compound of formula (I), or a corresponding amount of the pharmaceutically acceptable salt thereof;
about 14-22 mg silicified microcrystalline cellulose;
about 48-73 mg hypromellose K4M;
about 38-58 mg sodium lauryl sulfate;
about 0.1-3 mg colloidal silicone dioxide; and
about 1-5 mg magnesium stearate;
(K1)
about 88-133 mg of the compound of formula (I), or a corresponding amount of the pharmaceutically acceptable salt thereof;
about 21-32 mg silicified microcrystalline cellulose;
about 72-110 mg hypromellose K100M;
about 58-86 mg sodium lauryl sulfate;
about 0.1-3 mg colloidal silicone dioxide; and
about 3-6 mg magnesium stearate;
(L)
about 88-133 mg of the compound of formula (I), or a corresponding amount of the pharmaceutically acceptable salt thereof;
about 21-32 mg silicified microcrystalline cellulose;
about 72-110 mg hypromellose K4M;
about 58-86 mg sodium lauryl sulfate;
about 0.1-3 mg colloidal silicone dioxide; and
about 3-6 mg magnesium stearate;
(M)
about 118-178 mg of the compound of formula (I), or a corresponding amount of the pharmaceutically acceptable salt thereof;
about 28-43 mg silicified microcrystalline cellulose;
about 96-145 mg hypromellose K4M;
about 76-116 mg sodium lauryl sulfate;
about 0.1-3 mg colloidal silicone dioxide; and
about 4-8 mg magnesium stearate;
(N)
about 118-178 mg of the compound of formula (I), or a corresponding amount of the pharmaceutically acceptable salt thereof;
about 28-43 mg silicified microcrystalline cellulose;
about 96-145 mg hypromellose K100LV;
about 76-116 mg sodium lauryl sulfate;
about 0.1-3 mg colloidal silicone dioxide; and
about 4-8 mg magnesium stearate;
or (O)
about 98-148 mg of the compound of formula (I), or a corresponding amount of the pharmaceutically acceptable salt thereof;
about 23-36 mg silicified microcrystalline cellulose;
about 80-121 mg hypromellose K100LV;
about 64-96 mg sodium lauryl sulfate;
about 0.1-3 mg colloidal silicone dioxide; and
about 3-7 mg magnesium stearate.

100. The pharmaceutical formulation of claim 77, wherein the first layer comprises:
(e)
about 24.6 mg the compound of formula (I), or a corresponding amount of the pharmaceutically acceptable salt thereof,
about 98.70 mg silicified microcrystalline cellulose;
about 4.17 mg croscarmellose sodium;
about 4.91 mg sodium lauryl sulfate;
about 0.74 mg colloidal silicone dioxide; and
about 1.88 mg of magnesium stearate;
(f)
about 49.20 mg the compound of formula (I), or a corresponding amount of the pharmaceutically acceptable salt thereof,
about 73.54 mg silicified microcrystalline cellulose;
about 4.17 mg croscarmellose sodium;
about 4.91 mg sodium lauryl sulfate;
about 0.74 mg colloidal silicone dioxide; and
about 2.44 mg of magnesium stearate;
(g)
about 49.20 mg the compound of formula (I), or a corresponding amount of the pharmaceutically acceptable salt thereof,
about 69.88 mg silicified microcrystalline cellulose;
about 4.20 mg croscarmellose sodium;
about 9.60 mg sodium lauryl sulfate;
about 0.72 mg colloidal silicone dioxide; and
about 2.40 mg of magnesium stearate;
(h)
about 73.80 mg the compound of formula (I), or a corresponding amount of the pharmaceutically acceptable salt thereof,
about 104.82 mg silicified microcrystalline cellulose;
about 6.30 mg croscarmellose sodium;
about 14.40 mg sodium lauryl sulfate;
about 1.08 mg colloidal silicone dioxide; and
about 3.6 mg of magnesium stearate;
(i)
about 98.40 mg the compound of formula (I), or a corresponding amount of the pharmaceutically acceptable salt thereof,
about 139.76 mg silicified microcrystalline cellulose;
about 8.40 mg croscarmellose sodium;
about 19.20 mg sodium lauryl sulfate;
about 1.44 mg colloidal silicone dioxide; and
about 4.8 mg of magnesium stearate;
or (j)
about 123.00 mg the compound of formula (I), or a corresponding amount of the pharmaceutically acceptable salt thereof;
about 174.70 mg silicified microcrystalline cellulose;
about 10.50 mg croscarmellose sodium;
about 24.00 mg sodium lauryl sulfate;
about 1.80 mg colloidal silicone dioxide; and
about 6.00 mg of magnesium stearate;
and wherein the second layer comprises:
(G)
about 36.90 mg the compound of formula (I), or a corresponding amount of the pharmaceutically acceptable salt thereof,
about 52.00 mg silicified microcrystalline cellulose;
about 49.20 mg hypromellose K100M;
about 24.00 mg of sodium lauryl sulfate;
about 0.90 mg of colloidal silicone dioxide; and
about 2.00 mg of magnesium stearate;
(H)
about 73.80 mg of the compound of formula (I), or a corresponding amount of the pharmaceutically acceptable salt thereof;
about 14.62 mg silicified microcrystalline cellulose;
about 49.20 mg hypromellose K100M;
about 24.00 mg sodium lauryl sulfate;
about 0.90 mg colloidal silicone dioxide; and
about 2.48 mg magnesium stearate;
(I)
about 73.80 mg of the compound of formula (I), or a corresponding amount of the pharmaceutically acceptable salt thereof;
about 17.76 mg silicified microcrystalline cellulose;
about 60.36 mg hypromellose K100M;
about 48.00 mg sodium lauryl sulfate;
about 1.08 mg colloidal silicone dioxide; and
about 3.00 mg magnesium stearate;
(J)
about 73.80 mg of the compound of formula (I), or a corresponding amount of the pharmaceutically acceptable salt thereof;
about 17.76 mg silicified microcrystalline cellulose;
about 60.36 mg hypromellose K4M;
about 48.00 mg sodium lauryl sulfate;
about 1.08 mg colloidal silicone dioxide; and
about 3.00 mg magnesium stearate;
(K1)
about 110.70 mg of the compound of formula (I), or a corresponding amount of the pharmaceutically acceptable salt thereof;
about 26.64 mg silicified microcrystalline cellulose;
about 90.54 mg hypromellose K100M;
about 72.00 mg sodium lauryl sulfate;
about 1.62 mg colloidal silicone dioxide; and
about 4.50 mg magnesium stearate;
(L)
about 110.70 mg of the compound of formula (I), or a corresponding amount of the pharmaceutically acceptable salt thereof;
about 26.64 mg silicified microcrystalline cellulose;
about 90.54 mg hypromellose K4M;
about 72.00 mg sodium lauryl sulfate;
about 1.62 mg colloidal silicone dioxide; and
about 4.50 mg magnesium stearate;
(M)
about 147.60 mg of the compound of formula (I), or a corresponding amount of the pharmaceutically acceptable salt thereof;
about 35.52 mg silicified microcrystalline cellulose;
about 120.72 mg hypromellose K4M;
about 96.00 mg sodium lauryl sulfate;
about 2.16 mg colloidal silicone dioxide; and
about 6.00 mg magnesium stearate;
(N)
about 147.60 mg of the compound of formula (I), or a corresponding amount of the pharmaceutically acceptable salt thereof;
about 35.52 mg silicified microcrystalline cellulose;
about 120.72 mg hypromellose K100LV;
about 96.00 mg sodium lauryl sulfate;
about 2.16 mg colloidal silicone dioxide; and
about 6.00 mg magnesium stearate;
or (O)
about 123.00 mg of the compound of formula (I), or a corresponding amount of the pharmaceutically acceptable salt thereof;
about 29.60 mg silicified microcrystalline cellulose;
about 100.60 mg hypromellose K100LV;
about 80.00 mg sodium lauryl sulfate;
about 1.80 mg colloidal silicone dioxide; and
about 5.00 mg magnesium stearate.

101. The pharmaceutical formulation of claim 77, wherein the pharmaceutical formulation is a bilayer tablet, and wherein:
(i-a) the first part is an immediate-release layer comprising:
about 49.2 mg of the compound of formula (I), or a corresponding amount of the pharmaceutically acceptable salt thereof,
about 51.8 mg of silicified microcrystalline cellulose;
about 3.4 mg of croscarmellose sodium;
about 4.0 mg of sodium lauryl sulfate;
about 0.6 mg of colloidal silicone dioxide; and
about 1.0 mg of magnesium stearate; and
(i-A) the second part is an extended-release layer comprising:
about 73.8 mg of the compound of formula (I), or a corresponding amount of the pharmaceutically acceptable salt thereof;
about 15.6 mg of silicified microcrystalline cellulose;
about 49.2 mg of hypromellose K100M;
about 24 mg of sodium lauryl sulfate;
about 0.9 mg of colloidal silicone dioxide; and
about 1.5 mg of magnesium stearate;
or
(ii-a) the first part is an immediate-release layer comprising:
about 49.2 mg of the compound of formula (I), or a corresponding amount of the pharmaceutically acceptable salt thereof;
about 51.8 mg of silicified microcrystalline cellulose;
about 3.4 mg of croscarmellose sodium;
about 4.0 mg of sodium lauryl sulfate;
about 0.6 mg of colloidal silicone dioxide; and
about 1.0 mg of magnesium stearate; and
(ii-A) the second part is an extended-release layer comprising:
about 73.8 mg of the compound of formula (I) or a corresponding amount of the pharmaceutically acceptable salt thereof,
about 33.6 mg of silicified microcrystalline cellulose;
about 49.2 mg of hypromellose K100M;
about 6 mg of sodium lauryl sulfate;
about 0.9 mg of colloidal silicone dioxide; and
about 1.5 mg of magnesium stearate;
or
(iii-a) the first part is an immediate-release layer comprising:
about 24.60 mg of the compound of formula (I), or a corresponding amount of the pharmaceutically acceptable salt thereof;
about 76.4 mg of silicified microcrystalline cellulose;
about 3.4 mg of croscarmellose sodium;
about 4.0 mg of sodium lauryl sulfate;
about 0.6 mg of colloidal silicone dioxide; and
about 1.0 mg of magnesium stearate; and
(iii-A) the second part is an extended-release layer, comprising:
about 36.90 mg of the compound of formula (I) or a corresponding amount of the pharmaceutically acceptable salt thereof;
about 52.5 mg of silicified microcrystalline cellulose;
about 49.20 mg of hypromellose K100M;
about 24.00 mg of sodium lauryl sulfate;
about 0.90 mg of colloidal silicone dioxide; and
about 1.5 mg of magnesium stearate;
or
(iv-a) the first part is an immediate-release layer comprising:
about 24.60 mg of the compound of formula (I), or a corresponding amount of the pharmaceutically acceptable salt thereof;
about 98.70 mg of silicified microcrystalline cellulose;
about 4.17 mg of croscarmellose sodium;
about 4.91 mg of sodium lauryl sulfate;
about 0.74 mg of colloidal silicone dioxide; and
about 1.88 mg of magnesium stearate; and
(iv-A) the second part is an extended-release layer comprising:
about 36.90 mg of the compound of formula (I) or a corresponding amount of the pharmaceutically acceptable salt thereof;
about 52.00 mg of silicified microcrystalline cellulose;
about 49.20 mg of hypromellose K100M;
about 24.00 mg of sodium lauryl sulfate;
about 0.90 mg of colloidal silicone dioxide; and
about 2.00 mg of magnesium stearate;
or
(v-a) the first part is an immediate-release layer comprising:
about 49.20 mg of the compound of formula (I), or a corresponding amount of the pharmaceutically acceptable salt thereof;
about 73.54 mg of silicified microcrystalline cellulose;
about 4.17 mg of croscarmellose sodium;
about 4.91 mg of sodium lauryl sulfate;
about 0.74 mg of colloidal silicone dioxide; and
about 2.44 mg of magnesium stearate; and
(v-A) the second part is an extended-release layer comprising:
about 73.80 mg of the compound of formula (I) or a corresponding amount of the pharmaceutically acceptable salt thereof;
about 14.62 mg of silicified microcrystalline cellulose;
about 49.20 mg of hypromellose K100M;
about 24.00 mg of sodium lauryl sulfate;
about 0.90 mg of colloidal silicone dioxide; and
about 2.48 mg of magnesium stearate;
or
(vi-a) the first part is an immediate-release layer comprising:
about 49.20 mg of the compound of formula (I), or a corresponding amount of the pharmaceutically acceptable salt thereof;
about 69.88 mg of silicified microcrystalline cellulose;
about 4.20 mg of croscarmellose sodium;
about 9.60 mg of sodium lauryl sulfate;
about 0.72 mg of colloidal silicone dioxide; and
about 2.40 mg of magnesium stearate; and
(vi-A) the second part is an extended-release layer comprising:
about 73.80 mg of the compound of formula (I) or a corresponding amount of the pharmaceutically acceptable salt thereof;
about 17.76 mg of silicified microcrystalline cellulose;
about 60.36 mg of hypromellose K100M or hypromellose K4M;
about 48.00 mg of sodium lauryl sulfate;
about 1.08 mg of colloidal silicone dioxide; and
about 3.00 mg of magnesium stearate;
or
(vii-a) the first part is an immediate-release layer comprising:
about 73.80 mg of the compound of formula (I), or a corresponding amount of the pharmaceutically acceptable salt thereof;
about 104.82 mg of silicified microcrystalline cellulose;
about 6.30 mg of croscarmellose sodium;
about 14.40 mg of sodium lauryl sulfate;
about 1.08 mg of colloidal silicone dioxide; and
about 3.60 mg of magnesium stearate;
(vii-A) the second part is an extended-release layer comprising:
about 110.70 mg of the compound of formula (I) or a corresponding amount of the pharmaceutically acceptable salt thereof;
about 26.64 mg of silicified microcrystalline cellulose;
about 90.54 mg of hypromellose K100M or hypromellose K4M;
about 72.00 mg of sodium lauryl sulfate;
about 1.62 mg of colloidal silicone dioxide; and
about 4.50 mg of magnesium stearate;
or
(viii-a) the first part is an immediate-release layer comprising:
about 98.40 mg of the compound of formula (I), or a corresponding amount of the pharmaceutically acceptable salt thereof;
about 139.76 mg of silicified microcrystalline cellulose;
about 8.40 mg of croscarmellose sodium;
about 19.20 mg of sodium lauryl sulfate;
about 1.44 mg of colloidal silicone dioxide; and
about 4.80 mg of magnesium stearate; and
(viii-A) the second part is an extended-release layer, comprising:
about 147.60 mg of the compound of formula (I) or a corresponding amount of the pharmaceutically acceptable salt thereof;
about 35.52 mg of silicified microcrystalline cellulose;
about 120.72 mg of hypromellose K4M;
about 96.00 mg of sodium lauryl sulfate;
about 2.16 mg of colloidal silicone dioxide; and
about 6.00 mg of magnesium stearate;
or
(ix-a) the first part is an immediate-release layer comprising:
about 98.40 mg of the compound of formula (I), or a corresponding amount of the pharmaceutically acceptable salt thereof;
about 139.76 mg of silicified microcrystalline cellulose;
about 8.40 mg of croscarmellose sodium;
about 19.20 mg of sodium lauryl sulfate;
about 1.44 mg of colloidal silicone dioxide; and
about 4.80 mg of magnesium stearate; and
(ix-A) the second part is an extended-release layer comprising:
about 147.60 mg of the compound of formula (I) or a corresponding amount of the pharmaceutically acceptable salt thereof;
about 35.52 mg of silicified microcrystalline cellulose;
about 120.72 mg of hypromellose K100LV;
about 96.00 mg of sodium lauryl sulfate;
about 2.16 mg of colloidal silicone dioxide; and
about 6.00 mg of magnesium stearate;
or
(x-a) the first part is an immediate-release layer comprising:
about 123.00 mg of the compound of formula (I), or a corresponding amount of the pharmaceutically acceptable salt thereof;
about 174.70 mg of silicified microcrystalline cellulose;
about 10.50 mg of croscarmellose sodium;
about 24.00 mg of sodium lauryl sulfate;
about 1.80 mg of colloidal silicone dioxide; and
about 6.00 mg of magnesium stearate; and
(x-A) the second part is an extended-release layer comprising:
about 123.00 mg of the compound of formula (I) or a corresponding amount of the pharmaceutically acceptable salt thereof;
about 29.60 mg of silicified microcrystalline cellulose;
about 100.60 mg of hypromellose K100LV;
about 80.00 mg of sodium lauryl sulfate;
about 1.80 mg of colloidal silicone dioxide; and
about 5.00 mg of magnesium stearate.

102. The pharmaceutical formulation of claim 77, wherein the pharmaceutical formulation is a film-coated bilayer tablet comprising coating agents selected from the group consisting of sodium carboxymethylcellulose, cellulose acetate, cellulose acetate phthalate, ethylcellulose, gelatin, pharmaceutical glaze, hydroxypropyl cellulose, hydroxypropylmethyl cellulose, hydroxypropyl methylcellulose phthalate, methacrylic acid copolymer, methylcellulose, polyethylene glycol, polyvinyl acetate phthalate, shellac, sucrose, titanium dioxide, carnauba wax, microcrystalline wax, zein, Polyvinyl Alcohol-Part. Hydrolyzed, Xanthan Gum, Gum Acacia, Macrogol (PEG) Polyvinyl Alcohol Graft Copolymer, and Opadry;
or wherein the coating agent is Opadry;
or wherein the Opadry is present in the amount of about 2-6% by weight of the core tablet.

103. The pharmaceutical formulation of claim 77, wherein the pharmaceutical formulation has a dissolution rate of >20% at 1 hour, a dissolution rate of >30% at 1 hour, or a dissolution rate of ≥40% at 1 hour.

104. The pharmaceutical formulation of claim 77, wherein the pharmaceutical formulation is administered once-a-day.

105. The pharmaceutical formulation of claim 77, wherein the compound of Formula (I) is used in the pharmaceutical formulation.

106. A pharmaceutical formulation, which corresponds to the first part (alternatively, first layer, or immediate-release layer) of the pharmaceutical formulation according to claim 1.

107. A pharmaceutical formulation, which corresponds to the second part (alternatively, second layer, or extended-release layer) of the pharmaceutical formulation according to claim 76.

108. A method for preventing, slowing the progression of, delaying, or treating one or more metabolic disorders selected from the group consisting of: diabetes (including type I, type II diabetes, type II diabetes with obesity, and cystic fibrosis-related diabetes (CFRD)), diabetic kidney disease, impaired glucose tolerance, impaired fasting blood glucose, hyperglycemia, postprandial hyperglycemia, overweight, obesity, hypertension, insulin resistance, diseases associated with islet dysfunction and/or metabolic syndrome; or improving blood glucose control and/or reducing fasting plasma glucose, postprandial plasma glucose and/or glycosylated hemoglobin HbA1c; or preventing, slowing, delaying, or reversing complications of diabetes mellitus; or curing diabetes, causing diabetes remission or regressing diabetes, comprising administering to a subject a therapeutically effective amount of the pharmaceutical formulation of claim 1.

109. A method for preventing, slowing the progression of, delaying, or treating one or more metabolic disorders selected from the group consisting of: diabetes (including type I, type II diabetes, type II diabetes with obesity, and cystic fibrosis-related diabetes (CFRD)), diabetic kidney disease, impaired glucose tolerance, impaired fasting blood glucose, hyperglycemia, postprandial hyperglycemia, overweight, obesity, hypertension, insulin resistance, diseases associated with islet dysfunction and/or metabolic syndrome; or improving blood glucose control and/or reducing fasting plasma glucose, postprandial plasma glucose and/or glycosylated hemoglobin HbA1c; or preventing, slowing, delaying, or reversing complications of diabetes mellitus; or curing diabetes, causing diabetes remission or regressing diabetes, comprising administering to a subject a therapeutically effective amount of the pharmaceutical formulation of claim 76.
